(19) United States
(12) Patent Application Publication
(10) Pub. No.: US 2025/0090524 A1
(43) Pub. Date: Mar. 20, 2025

(54) BTK INHIBITORS

(71) Applicant: BIOGEN MA INC., Cambridge, MA (US)

(72) Inventors: Brian T. Hopkins, Newton, MA (US); BIn Ma, Arlington, MA (US); Jürgen Schulz, Bonn (DE); Marta Nevalainen, Holliston, MA (US); TeYu Chen, Charlestown, MA (US); Robin J. Prince, Sharon, MA (US); Harold George Vandeveer, Stoneham, MA (US); Issac Marx, Arlington, MA (US); Simone Sciaboba, Wellesley, MA (US); Edward Yin Shiang Lin, Ashland, MA (US)

(21) Appl. No.: 18/709,268

(22) PCT Filed: Nov. 11, 2022
(86) PCT No.: PCT/US22/49712

§ 371 Date: May 10, 2024

Related U.S. Application Data
(60) Provisional application No. 63/278,718, filed on Nov. 12, 2021.

(51) Int. Cl. A61K 31/4985; A61K 31/437; A61K 31/55; C07D 471/04; C07D 487/04

(52) U.S. Cl. CPC A61K 31/4985; A61K 31/437; A61K 31/55; C07D 471/04; C07D 487/04

(57) ABSTRACT

Provided are compounds of Formula (I) or pharmaceutically acceptable salts thereof, wherein the variables in Formula (I) are as defined herein; and methods for their use and production.

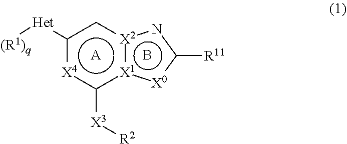

### RELATED APPLICATION

This application claims the benefit of the filing date, under 35 U.S.C. § 119(e), of U.S. Provisional Application No. 63/278,718, filed on Nov. 12, 2021, the entire contents of which are incorporated herein by reference.

### TECHNICAL FIELD

Provided are certain agents that inhibit Bruton's tyrosine kinase (Btk), and methods of making and using such agents.

### BACKGROUND

Protein kinases are a large multigene family consisting of more than 500 proteins which play a critical role in the development and treatment of a number of human diseases in oncology, neurology and immunology.

The Tec kinases are non-receptor tyrosine kinases which consists of five members (Tec (tyrosine kinase expressed in hepatocellular carcinoma), Btk (Bruton's tyrosine kinase), Itk (interleukin-2 (IL-2)-inducible T-cell kinase; also known as Emt or Tsk), Rlk (resting lymphocyte kinase; also known as Txk) and Bmx (bone-marrow tyrosine kinase gene on chromosome X; also known as Etk)) and are primarily expressed in haematopoietic cells, although expression of Bmx and Tec has been detected in endothelial and liver cells. Tec kinases (Itk, Rlk and Tec) are expressed in T cell and are all activated downstream of the T-cell receptor (TCR). Btk is a downstream mediator of B cell receptor (BCR) signaling which is involved in regulating B cell activation, proliferation, and differentiation. More specifically, Btk contains a PH domain that binds phosphatidylinositol (3,4,5)-trisphosphate (PIP3). PIP3 binding induces Btk to phosphorylate phospholipase C (PLCy), which in turn hydrolyzes PIP2 to produce two secondary messengers, inositol triphosphate (IP3) and diacylglycerol (DAG), which activate protein kinase PKC, which then induces additional B-cell signaling. Mutations that disable Btk enzymatic activity result in XLA syndrome (X-linked agammaglobulinemia), a primary immunodeficiency. Given the critical roles which Tec kinases play in both B-cell and T-cell signaling, Tec kinases are targets of interest for autoimmune disorders.

Given that Btk plays an important role in B-cell signaling, there is a great need in the art for effective inhibitors of Btk.

### SUMMARY

One aspect of the disclosure is a compound of Formula (I):

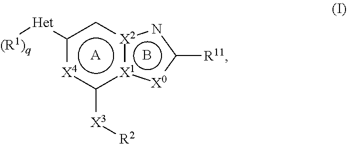

or a pharmaceutically acceptable salt thereof, wherein:
   X⁰ is N, X¹ is C, X² is N and X⁴ is N; X⁰ is CR⁰, X¹ is C, X² is N and X⁴ is N; X⁰ is CR⁰, X¹ is N, X² is C and X⁴ is N; X⁰ is CR⁰, X¹ is N, X² is C and X⁴ is CH; or X⁰ is CR⁰, X¹ is C, X² is N and X⁴ is CH;
   R⁰ is H, halo, —CH₃, halomethyl, cyclpropyl or CN;
   Het is phenyl, a 5-6 membered heteroaryl or a N—(C₁-C alkyl)pyridonyl;
   R¹ is H or C₁-C₃ alkyl, C₁-C₃ alkoxy, C₁-C₃ haloalkyl, C₃-C₆ cycloalkyl, or a 4-7 membered monocyclic oxygen containing heterocycle;
   X³ is absent, —S—, —SO₂—, CR^{3a}R^{3b}, —C(═O)— or —(C═O)—NH—*, where * indicates a point of attachment to R²;
   R^{3a} and R^{3b} are each independently H or halo, wherein at least one of R^{3a} and R^{3b} is not H;
   when X³ is absent, —S—, —SO₂—, CR^{3a}R^{3b}, or —(C═O)—NH—*, R² is a 4-12 membered mono or bicyclic nitrogen-containing heterocycle bonded to X³ through a ring carbon atom (“C-attached”), an 8-12 membered bicyclic nitrogen-containing heterocycle bonded to X³ through a ring nitrogen atom (“N-attached”), a 4-7 membered monocyclic oxygen containing heterocycle, phenyl, or a 3-12 membered monocyclic or bicyclic carbocyclyl, wherein the 4-7 membered monocyclic oxygen containing heterocycle, the phenyl and the 3-12 membered monocyclic or bicyclic carbocyclyl represented by R² are each substituted with a group represented by R⁴ and optionally further substituted with one or two groups represented by R¹⁰; the C-attached 4-12 membered mono or bicyclic nitrogen-containing heterocycle represented by R² is N-substituted with a group represented by R⁵ and optionally further substituted with one or two groups represented by R¹⁰; and the N-attached 8-12 membered bicyclic nitrogen-containing heterocycle represented by R² is N-substituted with a group represented by R⁵ and optionally further substituted with one or two groups represented by R¹⁰;
   when X³ is absent, R² can also be represented by formula (A):

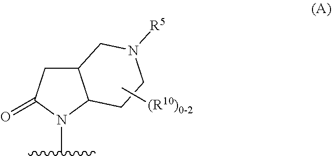

when X³ is —C(═O)—, R₂ is a 4-12 membered mono or bicyclic nitrogen-containing heterocycle bonded to X³ through a ring nitrogen atom (“N-attached”), a 4-7 membered monocyclic oxygen containing heterocycle, phenyl, or a 3-12 membered monocyclic or bicyclic carbocyclyl, wherein the 4-7 membered monocyclic oxygen containing heterocycle, the phenyl and the 3-12 membered monocyclic or bicyclic carbocyclyl represented by R² are each substituted with a group represented by R⁴ and optionally further substituted with one or two groups represented by R¹⁰; the N-attached 4-12 membered mono or bicyclic nitrogen-containing heterocycle represented by R² is C-substituted with a group represented by R⁴ and optionally further substituted with one or two groups represented by R¹⁰;
R⁴ is

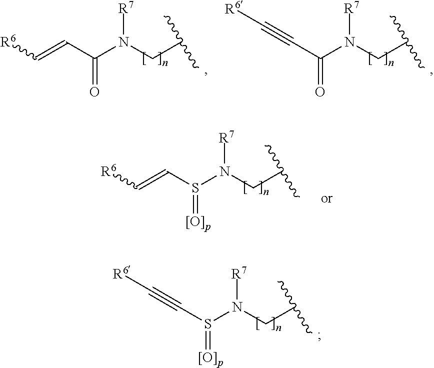

R⁵ is

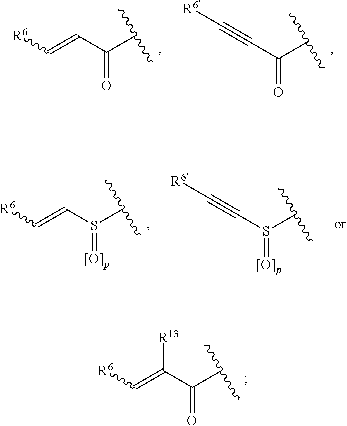

R⁶ is H, C₁-C₃ alkyl, C₁-C₃ haloalkyl, N(R^{a})₂ or CH₂N(R^{a})₂, wherein each R^{a} is independently H or methyl;
R^{6′} is H, C₁-C₃ alkyl or C₁-C₃ haloalkyl;
R⁷ is H, C₁-C₂ alkyl or C₁-C₂ fluoroalkyl;
each R¹⁰ is independently F or C₁₋₃alkyl;
R¹¹ is H or N(R¹²)₂;
each R¹² is independently H or C₁-C₃ alkyl;
R¹³ is CN or F;
n is 0 or 1;
p is 1 or 2; and
q is 1 or 2.

In a second aspect, the present disclosure provides a pharmaceutical composition comprising at least one compound described herein, or a pharmaceutically acceptable salt thereof, and a pharmaceutically acceptable excipient.

In a third aspect, the present disclosure provides methods of treating a disorder responsive to inhibition of Bruton's tyrosine kinase (Btk) in a subject. The methods comprise administering to the subject an effective amount of at least one compound described herein, or a pharmaceutically acceptable salt thereof.

The present disclosure also includes the use of at least one compound described herein, or a pharmaceutically acceptable salt thereof, for the manufacture of a medicament for the treatment of a disorder responsive to inhibition of Btk. Also provided is a compound described herein, or a pharmaceutically acceptable salt thereof for use in treating a disorder responsive to inhibition of Btk.

Other features or advantages will be apparent from the following detailed description of several embodiments, and also from the appended claims.

### DETAILED DESCRIPTION

The compounds or pharmaceutically acceptable salts thereof, as described herein, can have activity as Btk modulators. In particular, compounds or pharmaceutically acceptable salts thereof, as described herein, can be Btk inhibitors.

### I. Definitions

As used herein, the term “alkyl” refers to a fully saturated branched or unbranched hydrocarbon moiety. In some embodiments, the alkyl comprises 1 to 20 carbon atoms, 1 to 10 carbon atoms, 1 to 8 carbon atoms, 1 to 6 carbon atoms, or 1 to 4 carbon atoms. In some embodiments, an alkyl comprises from 6 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl.

“Alkenyl” refers to an unsaturated hydrocarbon group which may be linear or branched and has at least one carbon-carbon double bond. In some embodiments, alkenyl groups have 2 to 20 carbon atoms, 2 to 10 carbon atoms or 2-6 carbon atoms. The alkenyl group may contain 1, 2 or 3 carbon-carbon double bonds, or more. Examples of alkenyl groups include ethenyl, n-propenyl, iso-propenyl, n-but-2-enyl, n-hex-3-enyl and the like.

“Alkynyl” refers to an unsaturated hydrocarbon group which may be linear or branched and has at least one carbon-carbon triple bond. In some embodiments, alkynyl groups have 2 to 20 carbon atoms, 2 to 10 carbon atoms or 2-6 carbon atoms can be preferred. The alkynyl group may contain 1, 2 or 3 carbon-carbon triple bonds, or more. Examples of alkynyl groups include ethynyl, n-propynyl, n-but-2-ynyl, n-hex-3-ynyl and the like.

As used herein, the term “alkoxy” refers to a fully saturated branched or unbranched alkyl moiety attached through an oxygen bridge (i.e. a —O— C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. In some embodiments, alkoxy groups have about 1-4 carbons, more preferably about 1-2 carbons.

As used herein, the term “aryl” is defined to include all-carbon monocyclic or fused-ring polycyclic (i.e., rings which share adjacent pairs of carbon atoms) groups having a completely conjugated pi-electron system. An aryl group may have 6, 8, 9 or 10 carbon atoms in the ring(s). In some embodiments, an aryl group may have 6 or 10 carbon atoms in the ring(s). For example, as used herein, the term “(C₆-C₁₀)aryl” aromatic radicals containing from 6 to 10 carbon atoms such as phenyl, naphthyl, tetrahydronaphthyl, anthracenyl, indanyl and the like. An aryl group having 6 carbon atoms in the ring(s) may be optionally substituted by 1 to 5 suitable substituents.

In some embodiments, the number of carbon atoms in a group is specified herein by the prefix “Cₓ₋ₓₓ” or “Cₓ-Cₓₓ”, wherein x and xx are integers. For example, “C₁₋₄alkyl” or “C₁-C₄ alkyl” is an alkyl group which has from 1 to 4 carbon atoms.

As used herein, the term “carbocyclyl”, “carbocycle” or “carbocyclic ring” refers to a saturated or partially unsaturated monocyclic or bicyclic (e.g., fused, bridged or spiro ring systems) ring system which has from 4- to 12-ring members, all of which are carbon. The term “carbocyclyl” encompasses cycloalkyl groups and cycloalkenyl groups.

In one embodiment, the carbocyclyl is a 3- to 7-membered monocyclic carbocyclyl. Exemplary 3- to 7-membered monocyclic carbocyclyls include, but are not limited to, cyclopropyl, cyclobutyl, cyclopentyl, cyclohexyl, cycloheptyl, cyclopropenyl, cyclobutenyl, cyclopenentyl, cyclohexenyl, cycloheptenyl, cyclobutadienyl, cyclopentadienyl, cyclohexadienyl, cycloheptadienyl, phenyl and cycloheptatrienyl.

In one embodiment, the carbocyclyl is a 7- to 10-membered bicyclic carbocyclyl. Exemplary 7- to 10-membered bicyclic carbocyclyls include, but are not limited to, bicyclo[2.1.1]hexyl, bicyclo[2.2.1]heptyl, bicyclo[2.2.1]heptenyl, 6,6-dimethylbicyclo [3.1.1]heptyl, 2,6,6-trimethylbicyclo[3.1.1]heptyl, spiro[3.3]heptanyl, spiro[2.5]octanyl, spiro[2.2]pentanyl, spiro[3.3]heptanyl, bicyclo[3.3.0]octanyl, bicyclo[2.2.2]octanyl, bicyclo[3.3.1]nonanyl, bicyclo[3.3.2]decanyl, decalinyl, naphthyl and indanyl.

A fused bicyclic carbocyclyl has a 4 to 7 membered carbocycyl fused to a 3 to 7 membered non-aromatic carbocyclyl. Examples include decahydronapthalene, octahydro-1H-indene, octahydropentalene, decahydroazulene, decahydro-1H-annulene, bicycle[4.2.0]octane, bicycle[3.2.0]heptane and the like.

A bridged bicyclic carbocyclyl comprises a non-aromatic 5 to 7 membered carbocyclyl which shares three ring atoms with a 5 to 7 membered non-aromatic carbocyclyl. Examples of bridged bicyclics carbocycles include bicyclo[2.2.1]hepantyl, bicyclo[3.2.1]octanyl, and bicyclo[3.3.1]nonanyl.

“Cycloalkyl” refers to completely saturated monocyclic hydrocarbon groups of 3-7 carbon atoms, including cyclopropyl, cyclobutyl, cyclpentyl, cyclohexyl and cyclopentyl; and “cycloalkyenyl” refers to unsaturated non-aromatic monocyclic hydrocarbon groups of 3-7 carbon atoms, including cyclpenteneyl, cyclohexenyl and cyclopentenyl. The term “cycloalkyl” includes completely saturated monocyclic or bicyclic or spiro hydrocarbon groups of 3-7 carbon atoms, 3-6 carbon atoms, or 5-7 carbon atoms. In some embodiments, cycloalkyl is a 3- to 6-membered monocyclic cycloalkyl.

“Halogen” or “halo” may be fluoro, chloro, bromo or iodo.

The term “haloalkyl” or “halo-substituted alkyl” or refers to an alkyl group having at least one halogen substitution.

“Haloalkoxy” is a haloalkyl group which is attached to another moiety via an oxygen atom such as, e.g., but are not limited to —OCHCF₂ or —OCF₃.

“Heteroaryl” refers to an aromatic 5- to 6-membered monocyclic ring system, having 1 to 4 heteroatoms independently selected from O, N and S, and wherein N can be oxidized (e.g., N(O)) or quaternized, and S can be optionally oxidized to sulfoxide and sulfone.

Examples of 5- to 6-membered monocyclic heteroaryls include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, tetrazinyl, and the like. In one embodiment, a heteroaryl is a 5-membered heteroaryl. Examples of a 5-membered heteroaryl include, but are not limited to, pyrazolyl, oxazolyl, isoxazolyl, 1,2,3-oxadiazolyl, 1,3,4-oxadiazolyl, 1,2,4-oxadizolyl, 1,2,3-thiadiazolyl, 1,3,4-thiadiazolyl, 1,2,4-thiadiazolyl, 1,2,3-triazolyl, 1,2,4-triazolyl, and tetrazolyl.

The terms “heterocyclyl” and “heterocycle” refer to a saturated or partially unsaturated monocyclic or bicyclic (e.g., fused, bridged or spiro ring systems) ring system which has from 3- to 12-ring members, at least one of which is a heteroatom, and up to 4 (e.g., 1, 2, 3, or 4) of which may be heteroatoms, wherein the heteroatoms are independently selected from O, S and N, and wherein C can be oxidized (e.g., C(O)), N can be oxidized (e.g., N(O)) or quaternized, and S can be optionally oxidized to sulfoxide and sulfone. In some embodiments, the heterocyclyl is a 4- to 6-membered, 4- to 7-membered or 3- to 7-membered monocyclic heteterocycle. In some embodiments, the heterocyclyl is a 7- to 12-membered bicyclic heterocycle, which can be fused, bridged or spiro bicyclic heterocycle. In some embodiments, the bicyclic heterocycle may include a non-aromatic heterocycle fused to a heteroaromatic ring.

Examples of monocyclic heterocycle include, but are not limited to, oxetanyl, thietanyl, azetedinyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl.

Examples of bicyclic heterocycle include, but are not limited to, 9-azabicyclo[3.3.1]nonanyl, 2-azabicyclo[2.2.2]octanyl, 8-azabicyclo[3.2.1]octanyl, 2,7-diaza spiro[4.4]nonane, octahydrocyclopenta[c]pyrrolyl, octahydro-1H-pyrrolo[3,4-c]pyridine.

A “fused ring system” has from 8 to 12 members (ring atoms) and two rings which share two adjacent ring atoms. A fused bicyclic heterocycle has a 4 to 7 membered heterocycle fused to a 4 to 7 membered heterocycle or a 3 to 7 membered carbocyclyl. A fused bicyclic heterocyclyl can also have a 4 to 7 membered heterocycle fused to a 5 to 6 membered heteroaryl. Examples include cyclopentapyrrolidinyl, cyclopentapiperidinyl, cyclopentaazapanyl, cyclohexapyrrolidinyl, cyclohexapiperidinyl, cyclohexaazapanyl, cycloheptapyrrolidinyl, cycloheptapiperidinyl, cycheptaazapanyl, pyrrolopyrrolidinyl, pyrrolopiperidinyl, pyrroloazapanyl, furanopyrrolidinyl, furanopiperidinyl, furanoazapanyl, pyranopyrrolidinyl, pyranopiperidinyl, pyranoazapanyl, dihydropyrrolo[3,4-d]thiazoyl and the like.

A “bridged bicyclic ring system” (also referred to herein as a “bridged bicyclic” or “bridged ring system”) has 7 to 10 members (ring atoms) and two rings which share three adjacent ring atoms. A bridged bicyclic heterocycle comprises a 5 to 7 membered heterocycle which shares three ring atoms with a 5 to 7 membered heterocycle or a 5 to 7 membered carbocycle. Examples nitrogen containing bridged bicyclics include azabicyclo[2.2.1]hepantyl, azabicyclo[3.2.1]octanyl, azabicyclo[3.3.1]nonanyl, diazabicyclo[2.2.1]hepantyl, diazabicyclo[3.2.1]octanyl and diazabicyclo[3.3.1]nonanyl.

Examples of oxygen containing bridged bicyclics include oxobicyclo[2.2.1]hepantyl, oxobicyclo[3.2.1]octanyl, oxobicyclo[3.3.1]nonanyl, oxa-azabicyclo[2.2.1]hepantyl, oxa-azabicyclo[3.2.1]octanyl and oxa-azabicyclo[3.3.1]nonanyl.

A “spiro ring system” (also referred to herein as a “spirocycle”) has 8 to 12 members (ring atoms) and two rings which share one ring atom. A spirobicyclic heterocycle comprises a 4 to 7 membered heterocycle which shares one atom with a 4 to 7 membered heterocycle or a 4 to 7 membered non-aromatic carbocycle. Examples of 8 to 12 nitrogen containing spiro rings systems include 3,4-azabicyclooctanyl, 4,4-azabicyclononanyl, 3,5-azabicyclononanyl, 3,6-azabicyclodecanyl, 4,5-azabicyclodecanyl, 3,7-azabicycloundecanyl, 4,6-azabicycloundecanyl and 5,5-azabicycloundecanyl. Examples of 8-12 oxygen containing spiro ring systems include 3,4-oxobicyclooctanyl, 4,4-oxobicyclononanyl, 3,5-oxobicyclononanyl, 3,6-oxobicyclodecanyl, 4,5-oxobicyclodecanyl, 3,7-oxobicycloundecanyl, 4,6-oxobicycloundecanyl and 5,5-xobicycloundecanyl.

Examples of 4 to 12 membered nitrogen containing heterocycles include pyrrolidinyl, imidazolidinyl, pyrazolidinyl, oxazolidinyl, isoxazolidinyl, thiazolidinyl, isothiazolidinyl, piperidinyl, piperazinyl, morpholinyl, thiomorpholinyl, azepanyl, oxepanyl, imidazolinyl, cyclopentapyrrolidinyl, cyclopentapiperidinyl, cyclopentaazapanyl, cyclohexapyrrolidinyl, cyclohexapyrrolidinyl, cyclohexaazapanyl, cycloheptapyrrolidinyl, cycloheptapyrrolidinyl, cycloheptaazapanyl, pyrrolopyrolidinyl, pyrrolopiperidinyl, pyrroloazapanyl, furanopiperidinyl, furanoazapanyl, pyranopyrrolidinyl, pyranopiperidinyl, pyranoazapanyl, azabicyclo[2.2.1]hepantyl, azabicyclo[3.2.1]octanyl, azabicyclo[3.3.1]nonanyl, diazabicyclo[2.2.1]hepantyl, diazabicyclo[3.2.1]octanyl, diazabicyclo[3.3.1]nonanyl, 3,4-azabicyclooctanyl, 4,4-azabicyclononanyl, 3,5-azabicyclononanyl, 3,6-azabicyclodecanyl, 4,5-azabicyclodecanyl, 3,7-azabicycloundecanyl, 4,6-azabicycloundecanyl and 5,5-azabicycloundecanyl. Examples of 4 to 7 membered nitrogen containing heterocycles (optionally containing one ring oxygen or one ring sulfur atom) include pyrrolidinyl, imidazolidinyl, pyrazolidinyl, oxazolidinyl, isoxazolidinyl, thiazolidinyl, isothiazolidinyl, piperidinyl, piperazinyl, morpholinyl, thiomorpholinyl, azepanyl, oxepanyl and imidazolinyl.

Examples of 4 to 7 membered oxygen containing heterocycles include oxetanyl, tetrahydrofuranyl, oxazolidinyl, isoxazolidinyl, dioxolanyl, oxathiolanyl, tetrahydropyranyl, morpholinyl, dioxanyl, oxepanyl, dihydrofuranyl and dihydropyranyl.

The suffic “yl” added to the end of a chemical name indicates that the named moiety is bonded to the molecule at point. The suffix “ene” added to the end of a chemical name indicates that the named moiety is bonded to the molecule at two points. Examples include azetidinylene, pyrrolindinylene, piperidinylene, azapanylene or oxazapanylene, which indicates that an azetidine, pyrrolidine, piperidine, azapane or oxazapane is bonded to the remainder of the compound at two points.

A nitrogen-containing heterocycle is “N-substituted” when a ring nitrogen atom is substituted

The term “oxo” refers to the diradical ═O.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds or pharmaceutically acceptable salts thereof as described herein, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase).

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a particular enantiomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired enantiomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers is at least 20%, 30%, 40%, 50%, 60%, 70%, 80%, 90%, 95%, 97%, 99%, 99.5% or 99.9%. The stereoisomeric purity the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated. In addition, some compounds may exhibit polymorphism.

In one embodiment, the invention provides deuterated compounds disclosed herein, in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance. The compounds or pharmaceutically acceptable salts thereof as described herein, may exist in tautomeric forms and mixtures and separate individual tautomers are contemplated.

### II. Compounds of the Disclosure

In a first embodiment, the compound of the present disclosure is represented by Formula (I) or a pharmaceutically acceptable salt thereof, wherein the variables are as described above.

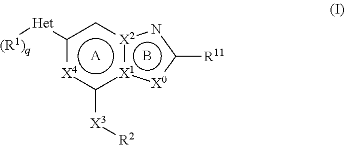

In a second embodiment of the present disclosure, for compounds of formula (I), or pharmaceutically acceptable salts thereof, R¹¹ is H; and the remaining variables are as described in the first embodiment.

In a third embodiment of the present disclosure, the compound is represented by one of the following formula:

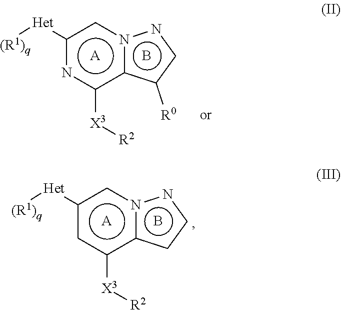

or a pharmaceutically acceptable salt thereof; and the remaining variables are as described in the first embodiment.

In a fourth embodiment of the present disclosure, the compound is represented by one of the following formula:

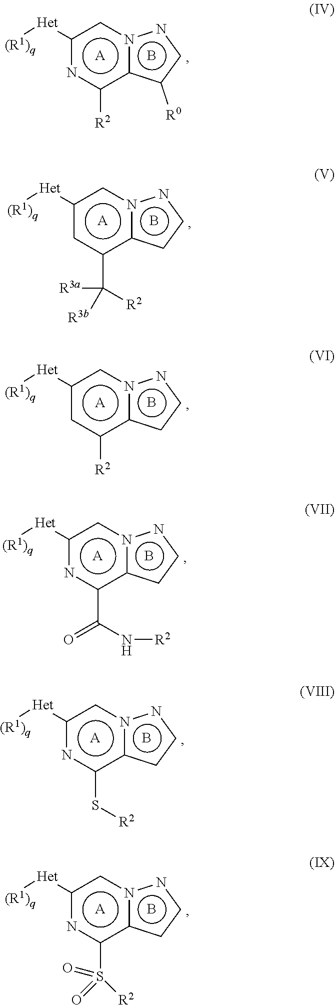

or a pharmaceutically acceptable salt thereof, wherein R^{3a} and R^{3b} are each independently H or halo, and at least one of R^{3a} and R^{3b} is not H; and the remaining variables are as described in the first embodiment.

In a fifth embodiment of the present disclosure, for compounds of formula (I), (II), (III), (IV), (V), (VI), (VII), (VIII) or (IX), or pharmaceutically acceptable salts thereof, R⁰ is H, C₁, F or —CH₃; and R^{3a} and R^{3b} are each F; and the remaining variables are as described in the first, second, third or fourth embodiment.

In a sixth embodiment of the present disclosure, for compounds of formula (I), (II), (III), (IV), (V), (VI), (VII), (VIII) or (IX), or pharmaceutically acceptable salts thereof, X³ is absent, —S—, —SO₂—, CR^{3a}R^{3b}, or —(C═O)—NH—*, and R² is a 4-9 membered monocyclic or bicyclic nitrogen-containing heterocycle bonded to X³ through a ring carbon atom (“C-attached”), an 8-9 membered bicyclic nitrogen-containing heterocycle bonded to X³ through a ring nitrogen atom (“N-attached”), phenyl, or a 4-6 membered monocyclic carbocyclyl, wherein the phenyl and 4-6 membered monocyclic carbocyclyl represented by R² are each substituted with a group represented by R⁴ and optionally further substituted with one or two groups represented by R¹⁰; the C-attached 4-9 membered monocyclic or bicyclic nitrogen-containing heterocycle represented by R² is N-substituted with a group represented by R⁵ and optionally further substituted with one or two groups represented by R¹⁰; and the N-attached 8-9 membered bicyclic nitrogen-containing heterocycle represented by R² is N-substituted with a group represented by R⁵ and optionally further substituted with one or two groups represented by R¹⁰; and the remaining variables are as described in the first, second, fourth or fifth embodiment.

In a seventh embodiment of the present disclosure, for compounds of formula (I), (II), (III), (IV), (V), (VI), (VII), (VIII) or (IX), or pharmaceutically acceptable salts thereof, R² is selected from cyclobutanyl, cyclopentanyl, cyclohexanyl and phenyl, each of which is substituted with a group represented by R⁴ and is optionally further substituted with one or two groups represented by R¹⁰; or R² is selected from azepanyl, azetidinyl, 9-azabicyclo[3.3.1]nonanyl, 2-azabicyclo[2.2.2]octanyl, 8-azabicyclo[3.2.1]octanyl, 2,7-diazaspiro[4.4]nonane, octahydrocyclopenta[c]pyrrolyl, octahydro-1H-pyrrolo[3,4-c]pyridine, piperidinyl, tetrahydropyridinyl and pyrrolidinyl, each of which is N-substituted with the group represented by R⁵ and optionally further substituted with the one or two groups represented by R¹⁰; and the remaining variables are as described in the sixth embodiment.

In an eighth embodiment of the present disclosure, for compounds of formula (I), (II), (III), (IV), (V), (VI), (VII), (VIII) or (IX), or pharmaceutically acceptable salts thereof, R² is selected from:

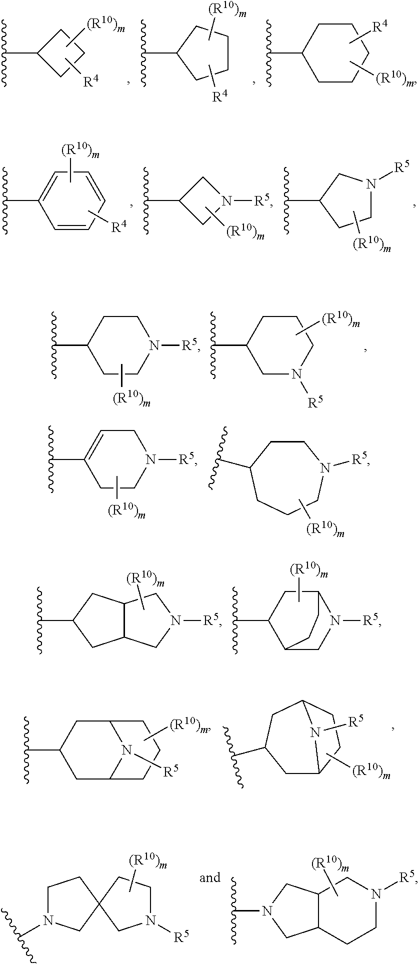

wherein m is 0, 1 or 2, and

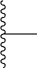

represents a bond to X³ or ring A; and the remaining variables are as described in the sixth embodiment.

In a ninth embodiment of the present disclosure, for compounds of formula (I), (II), (III), (IV), (V), (VI), (VII), (VIII) or (IX), or pharmaceutically acceptable salts thereof, R² is selected from:

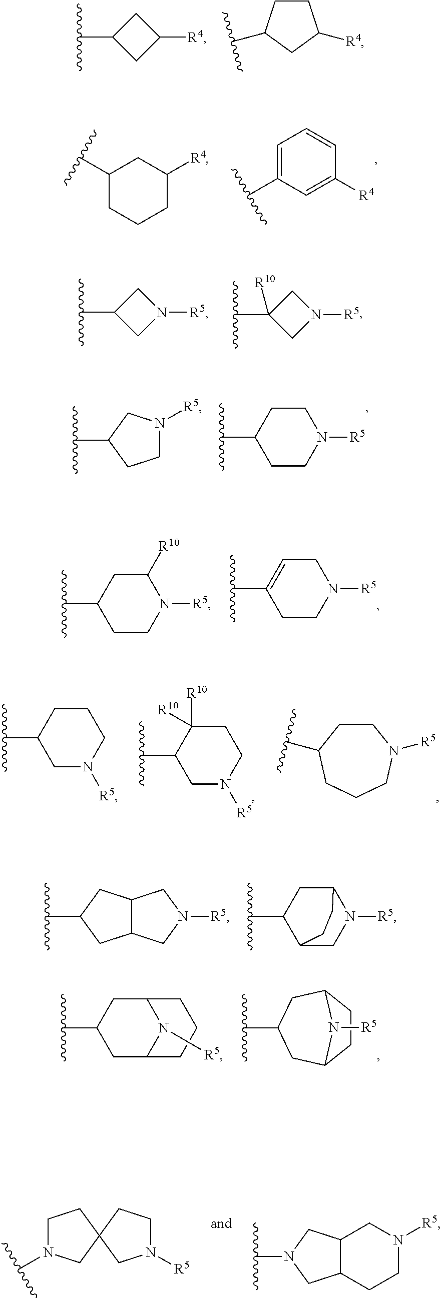

wherein

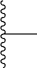

represents a bond to X or ring A; and the remaining variables are as described in the sixth embodiment.

In a tenth embodiment of the present disclosure, the compound is represented by the following formula:

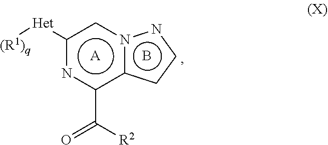

or a pharmaceutically acceptable salt thereof; and the remaining variables are as described in the first embodiment.

In an eleventh embodiment of the present disclosure, for compounds of formula (X), or pharmaceutically acceptable salts thereof, R² is a 4-7 membered monocyclic nitrogen-containing heterocycle bonded to X³ through a ring nitrogen atom (“N-attached”) or a 4-6 membered monocyclic carbocyclyl, wherein the 4-6 membered monocyclic carbocyclyl represented by R² are each substituted with a group represented by R⁴ and optionally further substituted with one or two groups represented by R¹⁰; and the N-attached 4-7 membered monocyclic nitrogen-containing heterocycle represented by R² is C-substituted with a group represented by R⁴ and optionally further substituted with one or two groups represented by R¹⁰; and the remaining variables are as described in the tenth embodiment.

In a twelfth embodiment of the present disclosure, for compounds of formula (X), or pharmaceutically acceptable salts thereof, R² is selected from:

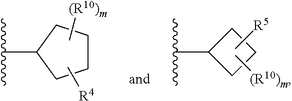

wherein m is 0, 1 or 2, and

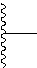

represents a bond to C(O)-ring A; and the remaining variables are as described in the tenth or eleventh embodiment.

In a thirteenth embodiment of the present disclosure, for compounds of formula (X), or pharmaceutically acceptable salts thereof, R² is selected from:

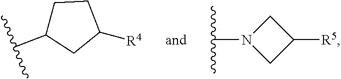

wherein

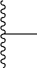

represents a bond to C(O)-ring A; and the remaining variables are as described in the tenth or eleventh embodiment.

In a fourteenth embodiment of the present disclosure, for compounds of formula (I), (II), (III), (IV), (V), (VI), (VII), (VIII), (IX) or (X), or pharmaceutically acceptable salts thereof, each R¹⁰ is independently F, —CH₃ or —CH₂CH₃; and the remaining variables are as described in the first, second, third, fourth, fifth, sixth, seventh, eighth, ninth, tenth, eleventh or twelfth embodiment.

In a fifteenth embodiment of the present disclosure, for compounds of formula (I), (II), (III), (IV), (V), (VI), (VII), (VIII), (IX) or (X), or pharmaceutically acceptable salts thereof,
R⁴ is:

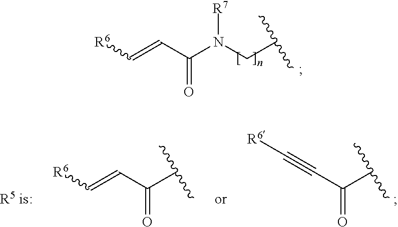

R⁶ is H or —CF₃; R^{6′} is —CH₃; R⁷ is H, —CH₃ or —CH₂CH₃; n is 0 or 1, and

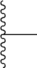

represents a bond to R²; and the remaining variables are as described in the first, second, third, fourth, fifth, sixth, seventh, eighth, ninth, tenth, eleventh, twelfth, thirteenth or fourteenth embodiment.

In a sixteenth embodiment of the present disclosure, for compounds of formula (I), (II), (III), (IV), (V), (VI), (VII), (VIII), (IX) or (X), or pharmaceutically acceptable salts thereof, Het is 5 membered heteroaryl; and the remaining variables are as described in the first, second, third, fourth, fifth, sixth, seventh, eighth, ninth, tenth, eleventh, twelfth, thirteenth, fourteenth, fifteenth or sixteenth embodiment.

In a seventeenth embodiment of the present disclosure, for compounds of formula (I), (II), (III), (IV), (V), (VI), (VII), (VIII), (IX) or (X), or pharmaceutically acceptable salts thereof, Het is pyrazolyl; and the remaining variables are as described in the first, second, third, fourth, fifth, sixth, seventh, eighth, ninth, tenth, eleventh, twelfth, thirteenth, fourteenth or fifteenth embodiment.

In an eighteenth embodiment of the present disclosure, for compounds of formula (I), (II), (III), (IV), (V), (VI), (VII), (VIII), (IX) or (X), or pharmaceutically acceptable salts thereof, Het is:

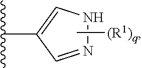

wherein

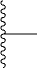

represents a bond to ring A; and the remaining variables are as described in the first, second, third, fourth, fifth, sixth, seventh, eighth, ninth, tenth, eleventh, twelfth, thirteenth, fourteenth or fifteenth embodiment.

In a nineteenth embodiment of the present disclosure, for compounds of formula (I), (II), (III), (IV), (V), (VI), (VII), (VIII), (IX) or (X), or pharmaceutically acceptable salts thereof, Het is:

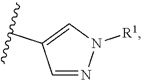

wherein

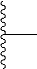

represents a bond to ring A; and the remaining variables are as described in the first, second, third, fourth, fifth, sixth, seventh, eighth, ninth, tenth, eleventh, twelfth, thirteenth, fourteenth or fifteenth embodiment.

In a twentieth embodiment of the present disclosure, for compounds of formula (I), (II), (III), (IV), (V), (VI), (VII), (VIII), (IX) or (X), or pharmaceutically acceptable salts thereof, R¹ is C₁-C₃ alkyl, C₁-C₃ haloalkyl or C₃-C₆ cycloalkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth or nineteenth embodiment.

In a twenty-first embodiment of the present disclosure, for compounds of formula (I), (II), (III), (IV), (V), (VI), (VII), (VIII), (IX) or (X), or pharmaceutically acceptable salts thereof, R¹ is —CH₃, —CF₃, cyclopropyl, or cyclobutyl; and the remaining variables are as described in the twentieth embodiment.

In a twenty-second embodiment of the present disclosure, the compound is represented by one of the following formula:

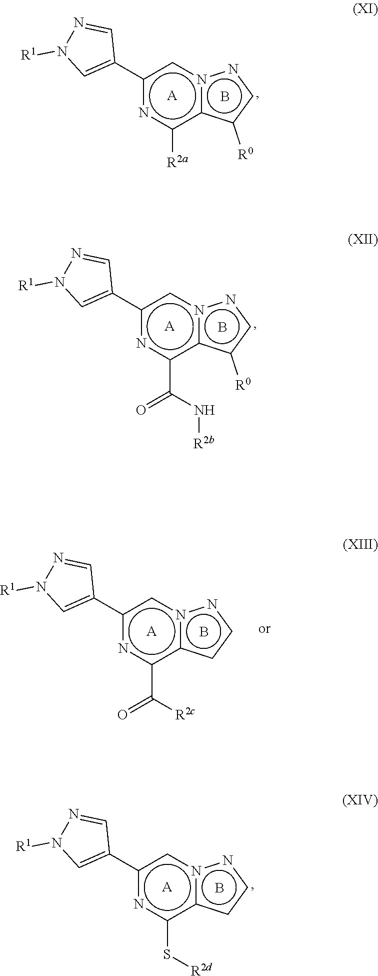

or a pharmaceutically acceptable salt thereof, wherein: R⁰ is H, F, or —CH₃; R¹ is —CH₃, cyclopropyl or cyclobutyl; R^{2a} is a 6-9 membered mono or bicyclic nitrogen-containing heterocycle bonded to X³ through a ring carbon atom (“C-attached”) which is N-substituted with a group represented by R⁵ and optionally further substituted with one or two groups represented by R¹⁰; R^{2b} is a 4-6 membered monocyclic nitrogen-containing heterocycle bonded to X³ through a ring carbon atom (“C-attached”) which is N-substituted with a group represented by R⁵ and optionally further substituted with one or two groups represented by R¹⁰; R^{2c} is a C₃₋₆cycloalkyl which is substituted with a group represented by R⁴ and optionally further substituted with one or two groups represented by R¹⁰; R^{2d} is a 4-7 membered monocyclic nitrogen-containing heterocycle bonded to X³ through a ring carbon atom (“C-attached”) which is N-substituted with a group represented by R⁵ and optionally further substituted with one or two groups represented by R¹⁰, or a C₃₋₆cycloalkyl which is substituted with a group represented by R⁴ and optionally further substituted with one or two groups represented by R¹⁰;
R⁴ is

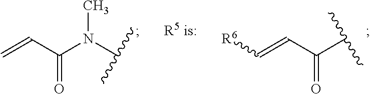

and R⁶ is H or CF₃.

In a twenty-third embodiment of the present disclosure, for compounds of formula (XI), (XII), (XIII) or (XIV), or pharmaceutically acceptable salts thereof, R^{2a} is 8-azabicyclo[3.2.1]octanyl, octahydrocyclopenta[c]pyrrolyl, or piperidinyl, each of which is N-substituted with the group represented by R⁵ and optionally further substituted with the one or two groups represented by R¹⁰; R^{2b} is azetidinyl or pyrrolidinyl, each of which is N-substituted with the group represented by R⁵ and optionally further substituted with the one or two groups represented by R¹⁰; R^{2c} is cyclopentyl substituted with a group represented by R⁴ and optionally further substituted with one or two groups represented by R¹⁰; and R^{2d} is azepanyl N-substituted with a group represented by R⁵ and optionally further substituted with one or two groups represented by R¹⁰, or cyclobutyl substituted with a group represented by R⁴ and optionally further substituted with one or two groups represented by R¹⁰; and the remaining variables are as described in the twenty-second embodiment.

In a twenty-fourth embodiment of the present disclosure, for compounds of formula for compounds of formula (XI), (XII), (XIII) or (XIV), or pharmaceutically acceptable salts thereof,
R^{2a} is

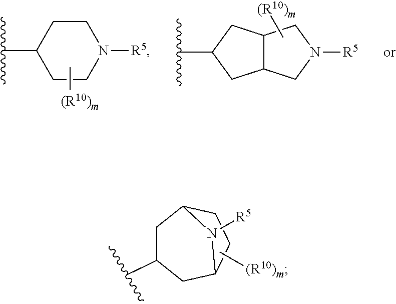

R^{2b} is

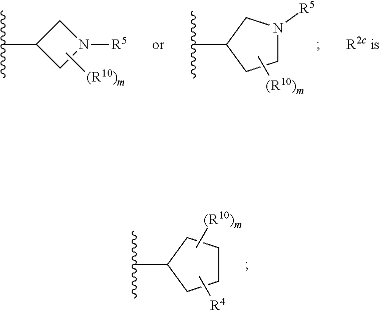

R^{2d} is

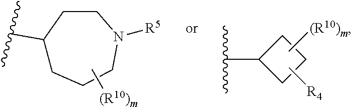

wherein m is 0, 1 or 2, and

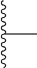

represents a bond to X³ or ring A; and the remaining variables are as described in the twenty-second embodiment.

In a twenty-fifth embodiment of the present disclosure, for compounds of formula for compounds of formula (XI), (XII), (XIII) or (XIV), or pharmaceutically acceptable salts thereof,
R^{2a} is

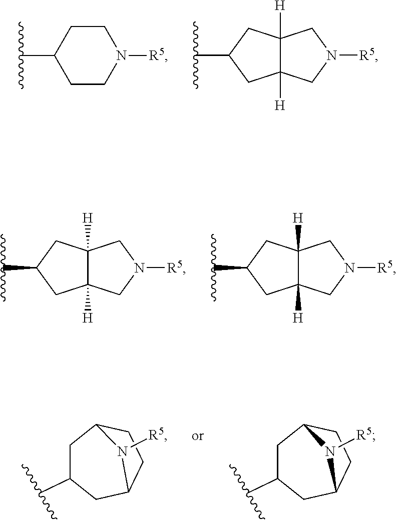

R^{2b} is

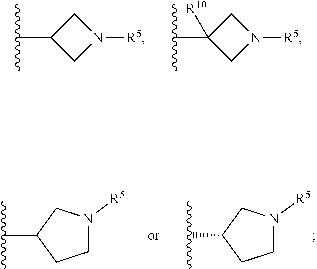

R^{2c} is

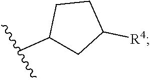

and
R^{2d} is

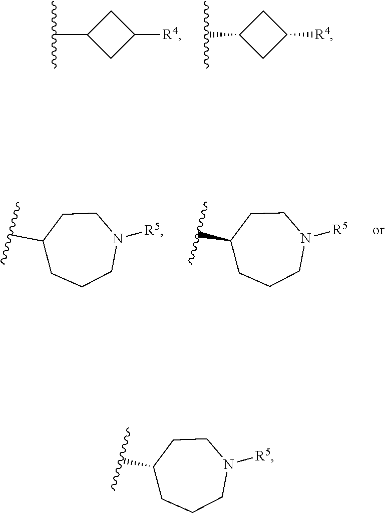

wherein R¹⁰ is —CH₃ or —CH₂CH₃, and

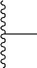

represents a bond to X³ or ring A; and the remaining variables are as described in the twenty-second embodiment.

The invention also includes both the neutral form and pharmaceutically acceptable salts of the compounds disclosed in the exemplification.

### III. Pharmaceutical Composition and Methods of Uses

Another embodiment is a pharmaceutical composition comprising at least one compound described herein, or a pharmaceutically acceptable salt thereof, and at least one pharmaceutically acceptable excipient and/or carrier.

The compounds, or pharmaceutically acceptable salts thereof described herein may be used to decrease the activity of Btk, or to otherwise affect the properties and/or behavior of Btk, e.g., stability, phosphorylation, kinase activity, interactions with other proteins, etc.

In some embodiments, the present invention provides methods of decreasing Btk enzymatic activity. In some embodiments, such methods include contacting a Btk with an effective amount of a Btk inhibitor. Therefore, the present invention further provides methods of inhibiting Btk enzymatic activity by contacting a Btk with a Btk inhibitor of the present invention.

One embodiment of the invention includes a method of treating a disorder responsive to inhibition of Btk in a subject comprising administering to the subject an effective amount of at least one compound described herein, or a pharmaceutically acceptable salt thereof.

The term “disorder responsive to inhibition of Btk” includes, for example, autoimmune disorders, inflammatory disorders, and cancers. In one embodiment, the present invention provides methods of treating autoimmune disorders, inflammatory disorders, and cancers in a subject in need thereof comprising administering to the subject an effective amount of at least one compound described herein, or a pharmaceutically acceptable salt thereof.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjogren's syndrome, temporal arteritis, rheumatoid arthritis, systemic lupus erythematosus and Wegener's granulomatosis.

The term “inflammatory disorders” includes diseases or disorders involving acute or chronic inflammation such as allergies, asthma, prostatitis, glomerulonephritis, pelvic inflammatory disease (PID), inflammatory bowel disease (IBD, e.g., Crohn's disease, ulcerative colitis), reperfusion injury, rheumatoid arthritis, atopic dermatitis, transplant rejection, and vasculitis. In some embodiments, the present invention provides a method of treating rheumatoid arthritis or lupus. In some embodiments, the present invention provides a method of treating multiple sclerosis.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present invention provides a method of treating leukemia or lymphoma.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or ‘treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The a compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

The invention is illustrated by the following examples, which are not intended to be limiting.

### IV. Exemplifications

### A. Abbreviations and Acronyms

Abbreviations and acronyms used herein include the following:
ABPR: automated back pressure regulator;
Ac₂O: acetic anhydride;
ACN: acetonitrile;
Aq.: aqueous;
Ar: argon;
Bn: benzyl;
Boc: tert-butoxy carbonyl;
Boc₂O: di-tert-butyl decarbonate;
BPin: pinacolatoboron;
(BPin)₂ or B₂pin₂: bis(pinacolato)diboron, i.e., 4,4,4′,4′,5,5,5′,5′—Octamethyl-2,2′-bi-1,3,2-dioxaborolane;
br: broad;
t-BuOH: tert butanol;
n-BuLi: n-butyl lithium;
° C.: degrees Celsius;
CHCl₃: chloroform;
CDCl₃: deutero-chloroform;
CDI: 1,1′-Carbonyldiimiidazole
CO₂: carbon dioxide;
Cs₂CO₃: cesium carbonate;
CsF: cesium fluoride;
CuL: copper iodide;
S: chemical shift;
d: doublet;
dd: double doublet;
ddd: double doublet of doublets;
DCM: dichloromethane;
DIEA or DIPEA: N-ethyldiisopropylamine or N,N-diisopropylethylamine;
DEA: diethylamine;
deg: degrees;
Dess-Martin periodinane or DMP: 1,1,1-Tris(acetyloxy)-1,1-dihydro-1,2-benziodoxol-3-(1H)-one;
DIAD: diisopropyl azodicarboxylate;
DABCO: 1,4-Diazabicyclo[2.2.2]octane
DABAL-Me₃: adduct of trimethylaluminum and DABCO
DME: 1,2-dimethoxyethane;
DMF: N,N-dimethylformamide;
DMP:
DMSO: dimethylsulfoxide;
DMSO-d₆: hexadeuterodimethyl sulfoxide;
DPPA: diphenylphosphoryl azide;
Et: ethyl;
Et₂O: ether;
EtOH: ethanol;
EA or EtOAc: ethyl acetate;
Eq.: equivalent;
g: gram;
h: hour;
HATU: O-(7-azabenzotriazole-1-yl)-1,1,3,3-tetramethyluronium hexafluorophosphate;
HE: heptanes;
HBr: hydrogen bromide;
HCl: hydrochloric acid;
HCO₂H: formic acid;
Hept: heptanes;
HFIP: hexafluoroisopropanol;
¹H NMR: proton nuclear magnetic resonance;
H₂O: water;
H₂SO₄: sulfuric acid;
HMPA: hexamethylphosphoramide;
HPLC: high pressure liquid chromatography;
Hz: Hertz;
IPA or iPrOH: iso-propanol;
J: coupling constant;
K₂CO₃: potassium carbonate;
kg: kilogram;
KHMDS: potassium hexamethldisilazide;
KOAc: potassium acetate;
KOH: potassium hydroxide;
KOt-Bu: potassium tert-butoxide
K₃PO₄: potassium phosphate tribasic;
K₄Fe(CN)₆·3H₂O: potassium hexacyanoferrate (II) trihydrate;
L: liter;
LCMS: liquid chromatography mass spectrometry;
LG: leaving group;
m: multiplet;
M: molar;
MBPR: manual back pressure regulator;
Me: methyl;
MeB(OH)₂: methylboronic acid;
MeCN: acetonitrile;
MeOH: methanol;
MeOH-d₄: deutero-methanol;
mg: milligram;
MgSO₄: magnesium sulfate;
MHz: mega Hertz;
mins: minutes;
mL: milliliters;
mmol: millimole;
MMPNO: methylmorpholine N-oxide;
mol: mole;
MS m/z: mass spectrum peak;
N₂: nitrogen;
NaOt-Bu: sodium tert-butoxide;
NaH: sodium hydride;
NaHCO₃: sodium bicarbonate;
NaHMDS: sodium hexamethyldisilylazide;
NaOH: sodium hydroxide;
Na2S₂O₃: sodium thiosulfate;
Na2SO₄: sodium sulfate;
NEt₃: triethylamine;
NFSI: N-fluorobenzenesulfonimide;
NH₃: ammonia;
NH₄C₁: ammonium chloride;
NH₄OH: ammonium hydroxide;
NH₄OAc: ammonium acetate;
NIS: N-iodosuccinimide;
OsO₄: osmium tetroxide;
P(cy)₃: tricyclohexylphosphine;
Pd₂(dba)₃: tris(dibenzylideneacetone)dipalladium (0);
Pd(dppf)Cl₂: [1,1′-bis(diphenylphosphino)ferrocene]dichloropalladium(II);
Pd(dppf)Cl₂-DCM: [1,1′-Bis(diphenylphosphino)ferrocene]dichloropalladium(II),
complex with dichloromethane
Pd(dtbpf)Cl₂: [1,1′-bis(di-tert-butylphosphino)ferrocene]dichloropalladium(II);
Pd(t-Bu₃P)₂: Bis(tri-tert-butylphosphine)palladium(0);
PE: petroleum ether
PEPPSI-IPr or Pd-PEPPSI-IPr: [1,3-bis(2,6-diisopropylphenyl)imidazol-2-ylidene](3-chloropyridyl)palladium(II) dichloride
PG: protecting group;
Ph: phenyl;
POCl₃: phosphoryl chloride;
Pyr: pyridine;
q: quartet;
Rf: retardation factor;
Rt: retention time;
rt or RT: room temperature;
Rh(OAc)₂ dimer: Rhodium(II) acetate dimer;
RuPhos: 2-dicyclohexylphosphino-2′,6′-diisopropoxybiphenyl;
s: singlet;
sat.: saturated;
SCX: strong cation exchange;
SFC: supercritical fluid chromatography;
SiO₂: silicon dioxide;
Si-SPE: silica solid phase extraction;
SPE: solid phase extraction;
t: triplet;
td: triple doublet;
t-BuONa: sodium tert-butoxide;
TEA: triethylamine;
TFA: trifluoroacetic acid;
Tf₂O: Trifluoromethanesulfonic anhydride;
THF: tetrahydrofuran;
TLC: thin layer chromatography;
TsNHNH₂: p-Toluenesulfonyl hydrazide;
T₃P: propanephosphonic acid anhydride;
μL: microliters;
μmol: micromole;
μW: microwave;
v/v: volume per volume;
Xphos: 2-dicyclohexylphosphino-2′,4′,6′-triisopropylbiphenyl;
Xphos G3: (2-dicyclohexylphosphino-2′,4′,6′-triisopropyl-1,1′-biphenyl)[2-(2′-amino-1,1′-biphenyl)]palladium(II) methanesulfonate;
tBuXPhos Pd G3: [(2-Di-tert-butylphosphino-2′,4′,6′-triisopropyl-1,1′-biphenyl)-2-(2′-amino-1,1′-biphenyl)]palladium(II) methanesulfonate

### B. Experimental

### Example 1: 1-[(3aS,6aR)-5-[6-(1-methylpyrazol-4-yl)pyrazolo[1,5-a]pyrazin-4-yl]-3,3a,4,5,6,6a-hexahydro-1H-cyclopenta[c]pyrrol-2-yl]prop-2-en-1-one

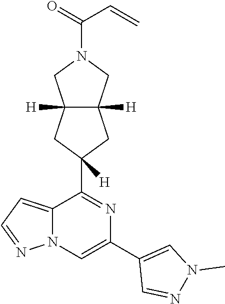 [SMILES: C=CC(=O)N1C[C@H]2C[C@@H](c3nc(-c4cnn(C)c4)cn4nccc34)C[C@H]2C1]

### 1. Preparation of tert-butyl 5-(trifluoromethylsulfonyloxy)-3,3a,6,6a-tetrahydro-1H-cyclopenta[c]pyrrole-2-carboxylate

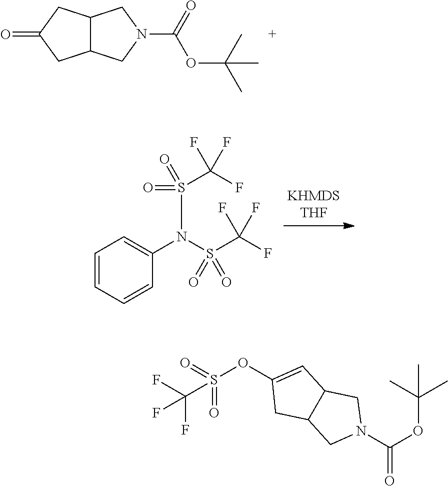 [SMILES: CC(C)(C)OC(=O)N1CC2CC(=O)CC2C1 ; O=S(=O)(N(c1ccccc1)S(=O)(=O)C(F)(F)F)C(F)(F)F ; CC(C)(C)OC(=O)N1CC2C=C(OS(=O)(=O)C(F)(F)F)CC2C1]

To a solution of tert-butyl 5-oxo-1,3,3a,4,6,6a-hexahydrocyclopenta[c]pyrrole-2-carboxylate (1.61 g, 7.15 mmol) in THF (20 mL) was cooled to −78° C. and [bis(trimethylsilyl)amino]potassium (1 M, 7.58 mL) was added dropwise. After stirred at 0° C. for 2 h, 1,1,1-trifluoro-N-phenyl-N-(trifluoromethylsulfonyl)methanesulfonamide (3.83 g, 10.72 mmol) was added in one pot. The mixture was allowed to warm to rt overnight. Aqueous NH₄Cl (sat'd) was added and extracted with EA. The organic layers were dried over sodium sulfate, filtered and concentrated. The residue was purified by silica gel column chromatography (EA/Hept.; 0-100%) to give tert-butyl 5-(trifluoromethylsulfonyloxy)-3,3a,6,6a-tetrahydro-1H-cyclopenta[c]pyrrole-2-carboxylate (2.2 g, 6.16 mmol, 86% yield) as a colorless oil. LCMS (ESI+): m/z calcd. for C₁₃H₁₉F₃NO₅S [M+H]+, 358.1; found, 358.1. Rt=0.98 min. ¹H NMR (400 MHz, CDCl₃, δ): 5.58 (d, J=1.5 Hz, 1H), 3.63-3.80 (m, 1H), 3.47-3.59 (m, 1H), 3.40 (qd, J=5.2, 2.4 Hz, 2H), 3.16 (br s, 1H), 2.85-3.00 (m, 2H), 2.40 (br d, J=14.8 Hz, 1H), 1.41-1.48 (m, 9H).

### 2. Preparation of tert-butyl 5-(4,4,5,5-tetramethyl-1,3,2-dioxaborolan-2-yl)-3,3a,6,6a-tetrahydro-1H-cyclopenta[c]pyrrole-2-carboxylate

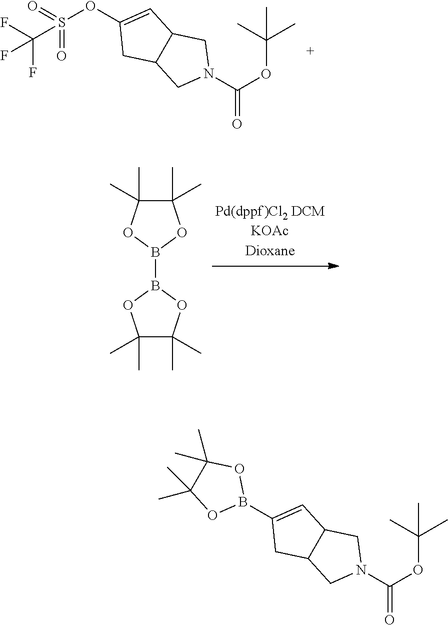

To a solution of tert-butyl 5-(trifluoromethylsulfonyloxy)-3,3a,6,6a-tetrahydro-1H-cyclopenta[c]pyrrole-2-carboxylate (606 mg, 1.70 mmol) and 4,4,5,5-tetramethyl-2-(4,4,5,5-tetramethyl-1,3,2-dioxaborolan-2-yl)-1,3,2-dioxaborolane (861.28 mg, 3.39 mmol) in Dioxane was added Pd(dppf)Cl₂·DCM (138.49 mg, 169.58 umol) and KOAc (499.29 mg, 5.09 mmol). The mixture was stirred at 90° C. under N₂ for 12 hours. After filtration through celite, the reaction mixture was concentrated under vacuum to give a crude, which was purified by silica gel column chromatography (PE/EA=7/3) to give tert-butyl 5-(4,4,5,5-tetramethyl-1,3,2-dioxaborolan-2-yl)-3,3a,6,6a-tetrahydro-1H-cyclopenta[c]pyrrole-2-carboxylate (386 mg, 1.15 mmol, 68% yield) as colorless oil. LCMS (ESI+): m/z calcd. for C₁₃H₂₃BNO₂[M+H—C₅H8O2]⁺, 236.2; found, 236.3. Rt=1.02 min. ¹H NMR (400 MHz, CDCl₃, δ): 6.33 (s, 1H), 3.57-3.67 (m, 1H), 3.47-3.54 (m, 1H), 3.41-3.46 (m, 1H), 3.34-3.40 (m, 1H), 2.93-2.98 (m, 1H), 2.83-2.92 (m, 1H), 2.57-2.70 (m, 1H), 2.28-2.44 (m, 1H), 1.42-1.46 (m, 9H), 1.27-1.28 (m, 12H).

### 3. Preparation of tert-butyl 5-[6-(1-methylpyrazol-4-yl)pyrazolo[1,5-a]pyrazin-4-yl]-3,3a,6,6a-tetrahydro-1H-cyclopenta[c]pyrrole-2-carboxylate

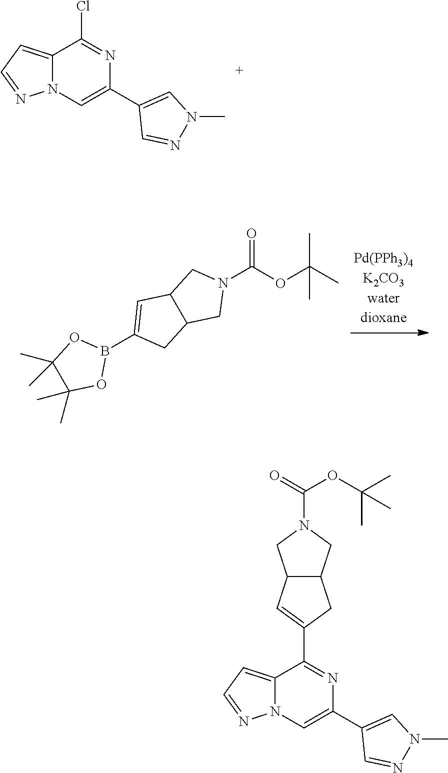 [SMILES: Cn1cc(-c2cn3nccc3c(Cl)n2)cn1 ; CC(C)(C)OC(=O)N1CC2C=C(B3OC(C)(C)C(C)(C)O3)CC2C1 ; Cn1cc(-c2cn3nccc3c(C3=CC4CN(C(=O)OC(C)(C)C)CC4C3)n2)cn1]

4-chloro-6-(1-methylpyrazol-4-yl)pyrazolo[1,5-a]pyrazine (200 mg, 855.96 umol), tert-butyl 5-(4,4,5,5-tetramethyl-1,3,2-dioxaborolan-2-yl)-3,3a,6,6a-tetrahydro-1H-cyclopenta[c]pyrrole-2-carboxylate (401.74 mg, 1.20 mmol), K₂CO₃ (354.91 mg, 2.57 mmol), Tetrakis(triphenylphosphine)palladium(0) (197.82 mg, 171.19 umol) in water (1 mL) and Dioxane (6 mL) was degassed and heated to 95° C. for 16 h. After cooling to room temperature, the mix was diluted with EtOAc and filtrated through celite. The concentrated residue was purified by silica gel column chromatography (DCM/EtOAc 0-100%) to give tert-butyl 5-[6-(1-methylpyrazol-4-yl)pyrazolo[1,5-a]pyrazin-4-yl]-3,3a,6,6a-tetrahydro-1H-cyclopenta[c]pyrrole-2-carboxylate (309 mg, 760.19 umol, 89% yield) as a white solid. LCMS (ESI+): m/z calcd. for C₂₂H₂₇N₆O₂ [M+H]⁺, 407.2; found, 407.3. Rt=0.82 min. ¹H NMR (400 MHz, CDCl₃, δ): 8.42-8.47 (m, 1H), 7.97-8.15 (m, 1H), 7.91 (d, J=7.4 Hz, 2H), 6.90 (d, J=2.0 Hz, 1H), 6.67 (s, 1H), 3.99 (s, 3H), 3.53-3.78 (m, 4H), 3.14-3.31 (m, 2H), 3.01-3.14 (m, 2H), 1.42-1.50 (m, 9H).

### 4. Preparation of 4-(1,2,3,3a,6,6a-hexahydrocyclopenta[c]pyrrol-5-yl)-6-(1-methylpyrazol-4-yl)pyrazolo[1,5-a]pyrazine

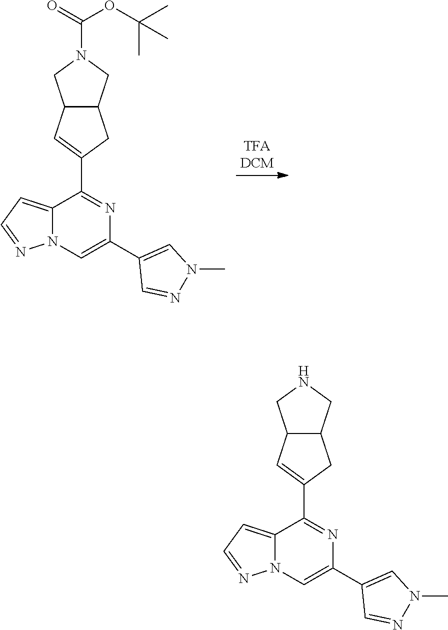 [SMILES: Cn1cc(-c2cn3nccc3c(C3=CC4CN(C(=O)OC(C)(C)C)CC4C3)n2)cn1 ; Cn1cc(-c2cn3nccc3c(C3=CC4CNCC4C3)n2)cn1]

tert-butyl 5-[6-(1-methylpyrazol-4-yl)pyrazolo[1,5-a]pyrazin-4-yl]-3,3a,6,6a-tetrahydro-1H-cyclopenta[c]pyrrole-2-carboxylate (309 mg, 760.19 umol) in DCM (4 mL) was added TFA (86.68 mg, 760.19 umol, 58.21 uL) and stirred at rt for 1 h. LCMS (ESI+): m/z calcd. for C₁₇H₁₉N₆[M+H]⁺, 307.2; found, 307.1. Rt=0.44 min. The crude was concentrated and used as is for the next step.

### 5. Preparation of 4-[(3aS,6aR)-1,2,3,3a,4,5,6,6a-octahydrocyclopenta[c]pyrrol-5-yl]-6-(1-methylpyrazol-4-yl)pyrazolo[1,5-a]pyrazine

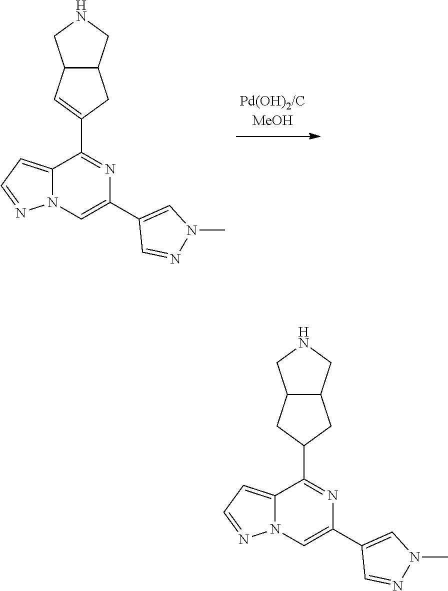 [SMILES: Cn1cc(-c2cn3nccc3c(C3=CC4CNCC4C3)n2)cn1 ; Cn1cc(-c2cn3nccc3c(C3CC4CNCC4C3)n2)cn1]

4-(1,2,3,3a,6,6a-hexahydrocyclopenta[c]pyrrol-5-yl)-6-(1-methylpyrazol-4-yl)pyrazolo[1,5-a]pyrazine (0.78 g, 1.71 mmol, Trifluoromethanesulfonic acid) in MeOH (10 mL) was added Pd(OH)₂/C (120.00 mg, 170.89 umol, 20% purity) and stirred under H₂ at 60 psi at rt for 2 days. After filtration through celite, the concentrated residue 4-[(3aS,6aR)-1,2,3,3a,4,5,6,6a-octahydrocyclopenta[c]pyrrol-5-yl]-6-(1-methylpyrazol-4-yl)pyrazolo[1,5-a]pyrazine (392 mg, 928.02 umol, 54% yield, TFA salt) was used as is for the next step. LCMS (ESI+): m/z calcd. for C₁₇H₂₁N₆[M+H]⁺, 309.2; found, 309.2. Rt=0.44 min.

### 6. Preparation of 1-[(3aS,6aR)-5-[6-(1-methylpyrazol-4-yl)pyrazolo[1,5-a]pyrazin-4-yl]-3,3a,4,5,6,6a-hexahydro-1H-cyclopenta[c]pyrrol-2-yl]prop-2-en-1-one

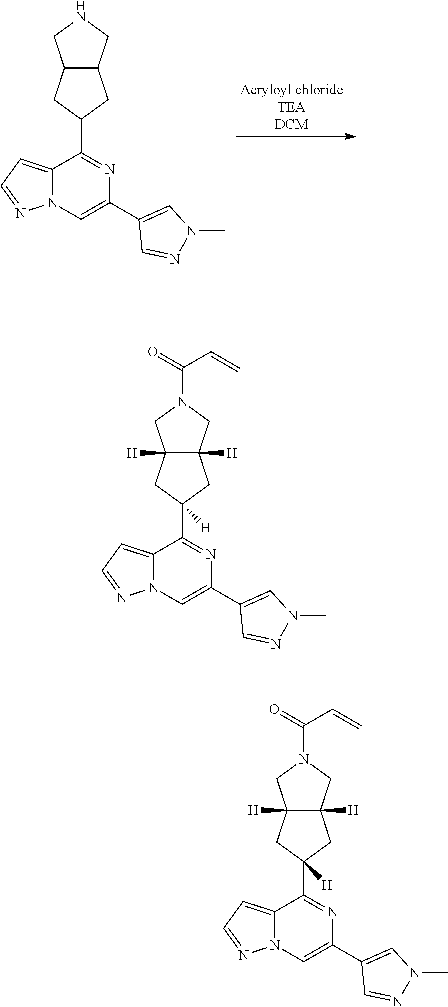 [SMILES: Cn1cc(-c2cn3nccc3c(C3CC4CNCC4C3)n2)cn1 ; C=CC(=O)N1C[C@H]2C[C@H](c3nc(-c4cnn(C)c4)cn4nccc34)C[C@H]2C1 ; C=CC(=O)N1C[C@H]2C[C@@H](c3nc(-c4cnn(C)c4)cn4nccc34)C[C@H]2C1]

4-[(3aS,6aR)-1,2,3,3a,4,5,6,6a-octahydrocyclopenta[c]pyrrol-5-yl]-6-(1-methylpyrazol-4-yl)pyrazolo[1,5-a]pyrazine (392 mg, 928.02 umol, TFA) in DCM (5 mL) was added TEA (187.81 mg, 1.86 mmol, 258.70 uL) and stirred for 5 min. After cooling to 0° C., acryloyl chloride (100.79 mg, 1.11 mmol, 90.48 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-15%) to give 1-[(3aS,6aR)-5-[6-(1-methylpyrazol-4-yl)pyrazolo[1,5-a]pyrazin-4-yl]-3,3a,4,5,6,6a-hexahydro-1H-cyclopenta[c]pyrrol-2-yl]prop-2-en-1-one (62.2 mg, 166.47 umol, 18% yield, 97% purity) as peak 2. LCMS (ESI+): m/z calcd. for C₂₀H₂₃N₆O [M+H]⁺, 363.2; found, 363.2. Rt=0.63 min. ¹H NMR (400 MHz, CDCl₃, δ): 8.42 (s, 1H), 7.95 (d, J=2.51 Hz, 1H), 7.92 (s, 1H), 7.87 (s, 1H), 6.72 (dd, J=0.88, 2.38 Hz, 1H), 6.51-6.37 (m, 2H), 5.69 (dd, 1H, J=2.5, 10.0 Hz), 3.99 (s, 3H), 3.85-3.76 (m, 4H), 3.64 (dd, 1H, J=4.4, 10.7 Hz), 3.01-2.84 (m, 2H), 2.47 (br t, 1H, J=7.9 Hz), 2.43 (br t, 1H, J=7.8 Hz), 2.17-2.01 (m, 2H).

### Example 2: 1-[(3aS,6aR)-5-[6-(1-methylpyrazol-4-yl)pyrazolo[1,5-a]pyrazin-4-yl]-3,3a,4,5,6,6a-hexahydro-1H-cyclopenta[c]pyrrol-2-yl]prop-2-en-1-one

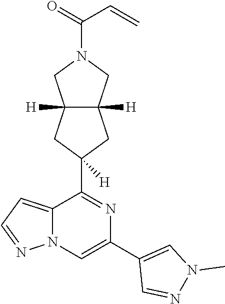 [SMILES: C=CC(=O)N1C[C@H]2C[C@H](c3nc(-c4cnn(C)c4)cn4nccc34)C[C@H]2C1]

1-[(3aS,6aR)-5-[6-(1-methylpyrazol-4-yl)pyrazolo[1,5-a]pyrazin-4-yl]-3,3a,4,5,6,6a-hexahydro-1H-cyclopenta[c]pyrrol-2-yl]prop-2-en-1-one was isolated with Example 1 as peak 1 (20 mg, 52.42 umol, 6% yield, 95% purity) as a white solid. LCMS (ESI+): m/z calcd. for C₂₀H₂₃N₆O [M+H]⁺, 363.2; found, 363.2. Rt=0.61 min. ¹H NMR (400 MHz, CDCl₃, δ): 8.41 (s, 1H), 7.94 (brs, 1H), 7.89 (s, 1H), 7.87 (s, 1H), 6.70 (brs, 1H), 6.52-6.37 (m, 2H), 5.71-5.68 (m, 1H), 3.97 (s, 3H), 3.87-3.48 (m, 5H), 3.07-2.98 (m, 2H), 2.53-2.41 (m, 2H), 2.07-1.99 (m, 2H).

### Example 3. 1-[5-[6-(1-methylpyrazol-4-yl)pyrazolo[1,5-a]pyridin-4-yl]-3,3a,4,5,6,6a-hexahydro-1H-cyclopenta[c]pyrrol-2-yl]prop-2-en-1-one

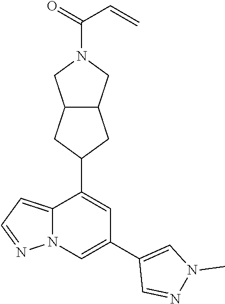 [SMILES: C=CC(=O)N1CC2CC(c3cc(-c4cnn(C)c4)cn4nccc34)CC2C1]

1. Preparation of tert-butyl 5-[6-(1-methylpyrazol-4-yl)pyrazolo[1,5-a]pyridin-4-yl]-3,3a,6,6a-tetrahydro-1H-cyclopenta[c]pyrrole-2-carboxylate

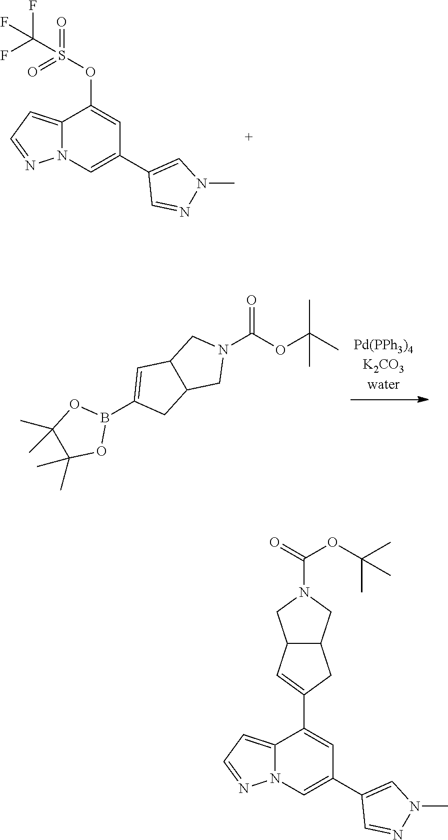 [SMILES: Cn1cc(-c2cc(OS(=O)(=O)C(F)(F)F)c3ccnn3c2)cn1 ; CC(C)(C)OC(=O)N1CC2C=C(B3OC(C)(C)C(C)(C)O3)CC2C1 ; Cn1cc(-c2cc(C3=CC4CN(C(=O)OC(C)(C)C)CC4C3)c3ccnn3c2)cn1]

[6-(1-methylpyrazol-4-yl)pyrazolo[1,5-a]pyridin-4-yl]trifluoromethanesulfonate (300 mg, 866.34 umol), tert-butyl 5-(4,4,5,5-tetramethyl-1,3,2-dioxaborolan-2-yl)-3,3a,6,6a-tetrahydro-1H-cyclopenta[c]pyrrole-2-carboxylate (435.65 mg, 1.30 mmol), K₂CO₃ (359.21 mg, 2.60 mmol), tetrakis(triphenylphosphine)palladium(0) (100.11 mg, 86.63 umol) in water (1 mL) and dioxane (6 mL) was degassed and heated to 95° C. for 16 h. After cooling to rt, the mix was diluted with EtOAc and filtrated through celite. The concentrated residue was purified by silica gel column chromatography (DCM/EtOAc 0-100%) to give tert-butyl 5-[6-(1-methylpyrazol-4-yl)pyrazolol[1,5-a]pyridin-4-yl]-3,3a,6,6a-tetrahydro-1H-cyclopenta[c]pyrrole-2-carboxylate (290 mg, 715.18 umol, 83% yield) as a white solid. LCMS (ESI+): m/z calcd. for C₂₃H₂₈N₅O₂ [M+H]⁺, 406.2; found, 406.2. Rt=0.85 min.

### 2. Preparation of 4-(1,2,3,3a,6,6a-hexahydrocyclopenta[c]pyrrol-5-yl)-6-(1-methylpyrazol-4-yl)pyrazolo[1,5-a]pyridine

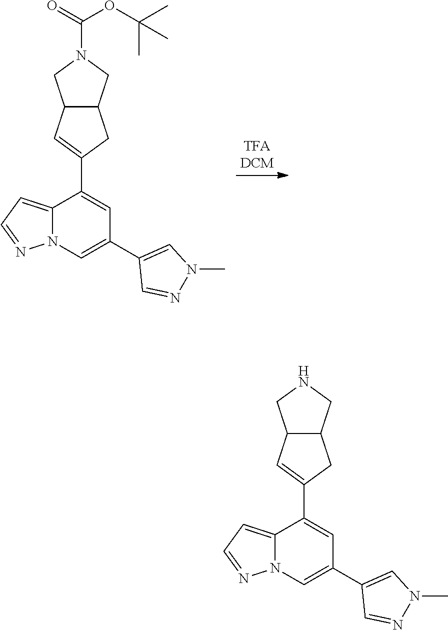 [SMILES: Cn1cc(-c2cc(C3=CC4CN(C(=O)OC(C)(C)C)CC4C3)c3ccnn3c2)cn1 ; Cn1cc(-c2cc(C3=CC4CNCC4C3)c3ccnn3c2)cn1]

Tert-butyl 5-[6-(1-methylpyrazol-4-yl)pyrazolo[1,5-a]pyridin-4-yl]-3,3a,6,6a-tetrahydro-1H-cyclopenta[c]pyrrole-2-carboxylate (290 mg, 715.18 umol) in DCM (4 mL) was added TFA (744.50 mg, 6.53 mmol, 0.5 mL) and stirred at rt for 1 h. LCMS (ESI+): m/z calcd. for C₁₈H₂₀N₅[M+H]⁺, 306.2; found, 306.2. Rt=0.52 min. The crude was concentrated and used as is for the next step.

### 3. Preparation of 4-[(3aS,6aR)-1,2,3,3a,4,5,6,6a-octahydrocyclopenta[c]pyrrol-5-yl]-6-(1-methylpyrazol-4-yl)pyrazolo[1,5-a]pyridine

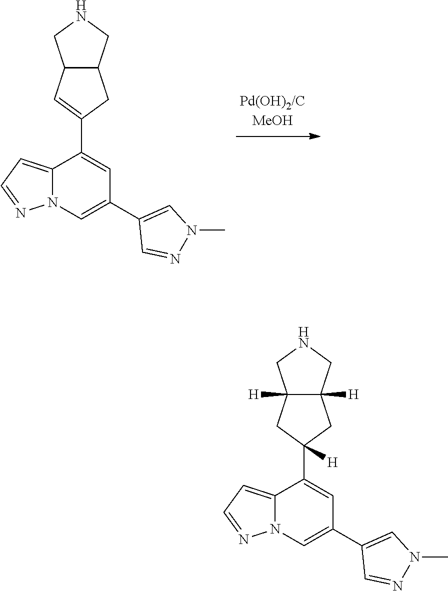 [SMILES: Cn1cc(-c2cc(C3=CC4CNCC4C3)c3ccnn3c2)cn1 ; Cn1cc(-c2cc([C@H]3C[C@H]4CNC[C@H]4C3)c3ccnn3c2)cn1]

4-(1,2,3,3a,6,6a-hexahydrocyclopenta[c]pyrrol-5-yl)-6-(1-methylpyrazol-4-yl)pyrazolo[1,5-a]pyridine (790 mg, 1.88 mmol, TFA) in MeOH (10 mL) was added Pd(OH)₂/C (132.27 mg, 188.36 umol, 20% purity) and stirred under H₂ at 60 psi at rt for 2 days. After filtration through celite, the concentrated residue 4-[(3aS,6aR)-1,2,3,3a,4,5,6,6a-octahydrocyclopenta[c]pyrrol-5-yl]-6-(1-methylpyrazol-4-yl)pyrazolo[1,5-a]pyridine (0.35 g, 765.08 umol, 41% yield, TFA) was used as is for the next step. LCMS (ESI+): m/z calcd. for C₁₈H₂₂N₅[M+H]⁺, 308.2; found, 308.1. Rt=0.51 min.

### 4. Preparation of 1-[5-[6-(1-methylpyrazol-4-yl)pyrazolo[1,5-a]pyridin-4-yl]-3,3a,4,5,6,6a-hexahydro-1H-cyclopenta[c]pyrrol-2-yl]prop-2-en-1-one

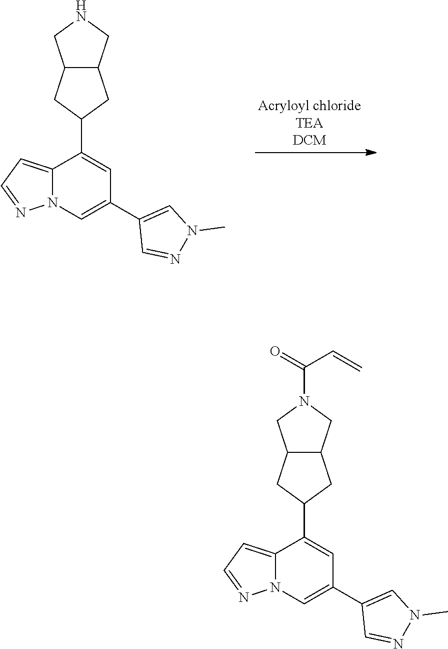 [SMILES: Cn1cc(-c2cc(C3CC4CNCC4C3)c3ccnn3c2)cn1 ; C=CC(=O)N1CC2CC(c3cc(-c4cnn(C)c4)cn4nccc34)CC2C1]

4-(1,2,3,3a,4,5,6,6a-octahydrocyclopenta[c]pyrrol-5-yl)-6-(1-methylpyrazol-4-yl)pyrazolo[1,5-a]pyridine (350 mg, 830.54 umol, TFA) in DCM (5 mL) was added TEA (168.08 mg, 1.66 mmol, 231.52 uL) and stirred for 5 min. After cooling to 0° C., Acryloyl chloride (90.21 mg, 996.64 umol, 80.97 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-50%) gave 1-[5-[6-(1-methylpyrazol-4-yl)pyrazolo[1,5-a]pyridin-4-yl]-3,3a,4,5,6,6a-hexahydro-1H-cyclopenta[c]pyrrol-2-yl]prop-2-en-1-one (89 mg, 233.93 umol, 28% yield, 95% purity). LCMS (ESI+): m/z calcd. for C₂₁H₂₄N₅O [M+H]⁺, 362.2; found, 362.2. Rt=0.62 min. ¹H NMR (400 MHz, CDCl₃, δ): 8.82 (br s, 1H), 7.99 (br s, 1H), 7.71-7.84 (m, 2H), 7.20-7.25 (m, 1H), 6.60 (br s, 1H), 6.38-6.53 (m, 2H), 5.70-5.74 (m, 1H), 3.96-4.03 (m, 3H), 3.75 (br d, J=6.0 Hz, 3H), 3.61 (br s, 1H), 3.39-3.56 (m, 1H), 2.95 (br s, 2H), 2.42-2.63 (m, 2H), 2.34 (br s, 2H).

### Example 4. 1-[4-[6-(1-methylpyrazol-4-yl)pyrazolo[1,5-a]pyrazin-4-yl]-1-piperidyl]prop-2-en-1-one

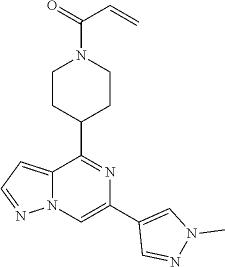 [SMILES: C=CC(=O)N1CCC(c2nc(-c3cnn(C)c3)cn3nccc23)CC1]

### 1. Preparation of tert-butyl 4-[6-(1-methylpyrazol-4-yl)pyrazolo[1,5-a]pyrazin-4-yl]-3,6-dihydro-2H-pyridine-1-carboxylate

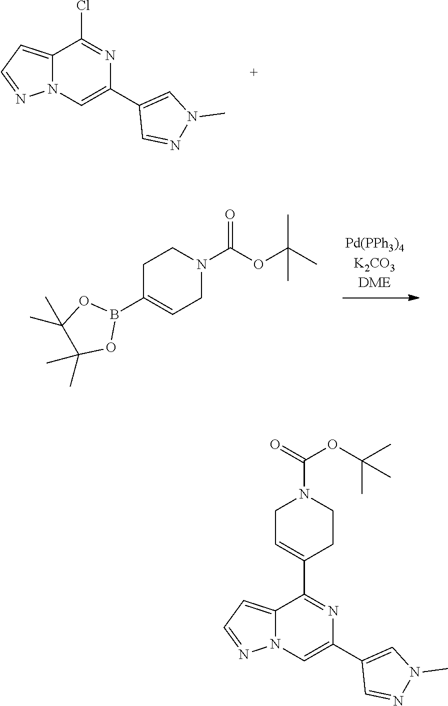 [SMILES: Cn1cc(-c2cn3nccc3c(Cl)n2)cn1 ; CC(C)(C)OC(=O)N1CC=C(B2OC(C)(C)C(C)(C)O2)CC1 ; Cn1cc(-c2cn3nccc3c(C3=CCN(C(=O)OC(C)(C)C)CC3)n2)cn1]

4-chloro-6-(1-methylpyrazol-4-yl)pyrazolo[1,5-a]pyrazine (214 mg, 915.87 umol), tert-butyl 4-(4,4,5,5-tetramethyl-1,3,2-dioxaborolan-2-yl)-3,6-dihydro-2H-pyridine-1-carboxylate (424.79 mg, 1.37 mmol), K₂CO₃ (379.75 mg, 2.75 mmol), tetrakis(triphenylphosphine) palladium(0) (211.67 mg, 183.17 umol) in 1,2-dimethoxyethane (5 mL) and water (1 mL) was degassed and heated to 95° C. for 16 h. After cooling to rt, the mix was diluted with EtOAc and filtrated through celite. The concentrated residue was purified by silica gel column chromatography (DCM/EtOAc 0-100%) to give tert-butyl 4-[6-(1-methylpyrazol-4-yl)pyrazolol[1,5-a]pyrazin-4-yl]-3,6-dihydro-2H-pyridine-1-carboxylate (144 mg, 378.51 umol, 41% yield) as a white solid. LCMS (ESI+): m/z calcd. for C₂₀H₂₅N₆O₂ [M+H]⁺, 381.2; found, 381.2. Rt=0.83 min.

### 2. Preparation of tert-butyl 4-[6-(1-methylpyrazol-4-yl)pyrazolo[1,5-a]pyrazin-4-yl]piperidine-1-carboxylate

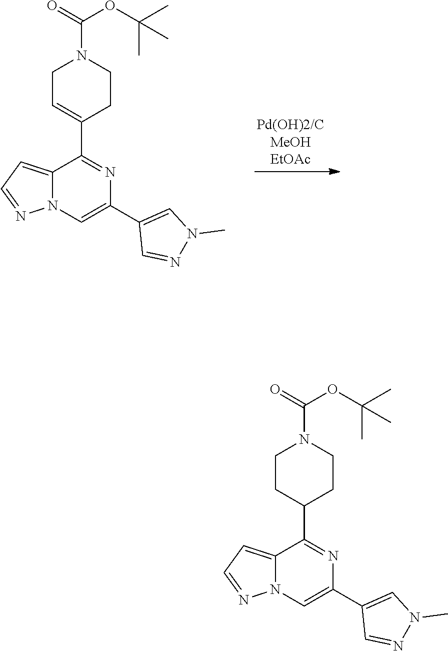 [SMILES: Cn1cc(-c2cn3nccc3c(C3=CCN(C(=O)OC(C)(C)C)CC3)n2)cn1 ; Cn1cc(-c2cn3nccc3c(C3CCN(C(=O)OC(C)(C)C)CC3)n2)cn1]

Pd(OH)₂/C (53.16 mg, 75.70 umol, 20% purity) in flask was added tert-butyl 4-[6-(1-methylpyrazol-4-yl)pyrazolo[1,5-a]pyrazin-4-yl]-3,6-dihydro-2H-pyridine-1-carboxylate (144 mg, 378.51 umol) in MeOH (5 mL) solution. The mix was stirred under 50 bar H₂ for 16 h. LCMS: Rt=0.88 min, m/z 384.2. After filtration through celite, the concentrated residue tert-butyl 4-[6-(1-methylpyrazol-4-yl)pyrazolo[1,5-a]pyrazin-4-yl]piperidine-1-carboxylate (143 mg, 373.90 umol, 99% yield) was used as is. LCMS (ESI+): m/z calcd. for C₂₀H27N₆O₂ [M+H]⁺, 383.2; found, 383.2. Rt=0.83 min.

### 3. Preparation of 6-(1-methylpyrazol-4-yl)-4-(4-piperidyl)pyrazolo[1,5-a]pyrazine

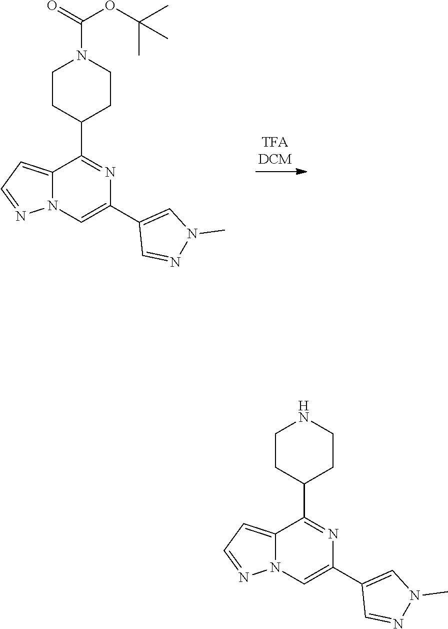 [SMILES: Cn1cc(-c2cn3nccc3c(C3CCN(C(=O)OC(C)(C)C)CC3)n2)cn1 ; Cn1cc(-c2cn3nccc3c(C3CCNCC3)n2)cn1]

tert-butyl 4-[6-(1-methylpyrazol-4-yl)pyrazolo[1,5-a]pyrazin-4-yl]piperidine-1-carboxylate (143 mg, 373.90 umol) in DCM was added TFA (42.63 mg, 373.90 umol, 28.63 uL) and stirred at for 1 h. After concentration, the crude residue was used as is for the next step. LCMS (ESI+): m/z calcd. for C₁₅H₁₉N₆[M+H]⁺, 283.2; found, 283.1. Rt=0.47 min.

### 4. Preparation of 1-[4-[6-(1-methylpyrazol-4-yl)pyrazolo[1,5-a]pyrazin-4-yl]-1-piperidyl]prop-2-en-1-one

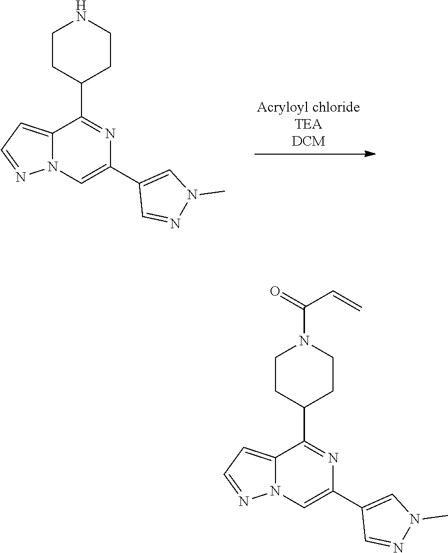 [SMILES: Cn1cc(-c2cn3nccc3c(C3CCNCC3)n2)cn1 ; C=CC(=O)N1CCC(c2nc(-c3cnn(C)c3)cn3nccc23)CC1]

6-(1-methylpyrazol-4-yl)-4-(4-piperidyl)pyrazolo[1,5-a]pyrazine (269 mg, 678.67 umol, TFA salt) in DCM (4 mL) was added TEA (137.35 mg, 1.36 mmol, 189.19 uL) and stirred for 5 min. After cooling to 0° C., acryloyl chloride (73.71 mg, 814.40 umol, 66.17 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-15%) then prep HPLC to give 1-[4-[6-(1-methylpyrazol-4-yl)pyrazolo[1,5-a]pyrazin-4-yl]-1-piperidyl]prop-2-en-1-one (38.7 mg, 109.29 umol, 16% yield, 95% purity) as a white solid. LCMS (ESI+): m/z calcd. for C₁₈H₂₁N₆O [M+H]⁺, 337.2; found, 337.1. Rt=0.58 min. ¹H NMR (400 MHz, CDCl₃, δ): 8.45-8.47 (m, 1H), 7.99-8.01 (m, 1H), 7.96-7.99 (m, 1H), 7.89-7.91 (m, 1H), 6.78-6.81 (m, 1H), 6.62-6.70 (m, 1H), 6.30-6.38 (m, 1H), 5.71-5.76 (m, 1H), 4.79-4.92 (m, 1H), 4.17-4.26 (m, 1H), 4.00 (s, 3H), 3.42-3.53 (m, 1H), 3.29-3.40 (m, 1H), 2.93-3.02 (m, 1H), 2.06-2.11 (m, 4H).

### Example 5. 1-[3-[6-(1-methylpyrazol-4-yl)pyrazolo[1,5-a]pyrazin-4-yl]-1-piperidyl]prop-2-en-1-one

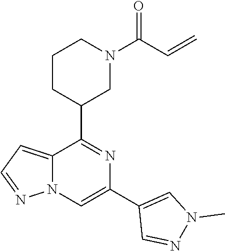 [SMILES: C=CC(=O)N1CCCC(c2nc(-c3cnn(C)c3)cn3nccc23)C1]

### 1. Preparation of benzyl 5-[6-(1-methylpyrazol-4-yl)pyrazolo[1,5-a]pyrazin-4-yl]-3,4-dihydro-2H-pyridine-1-carboxylate

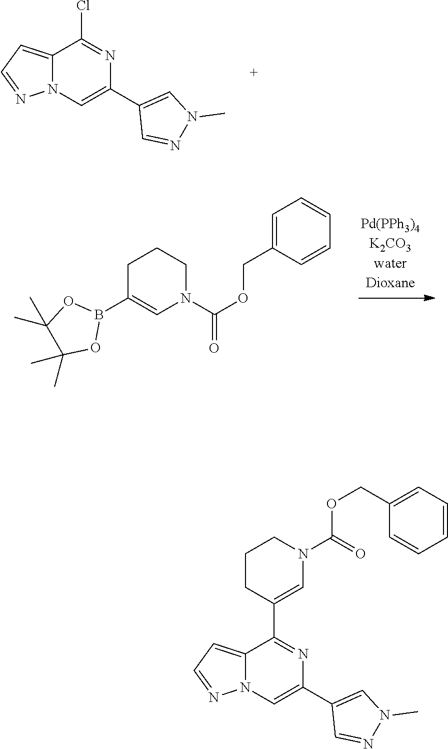 [SMILES: Cn1cc(-c2cn3nccc3c(Cl)n2)cn1 ; CC1(C)OB(C2=CN(C(=O)OCc3ccccc3)CCC2)OC1(C)C ; Cn1cc(-c2cn3nccc3c(C3=CN(C(=O)OCc4ccccc4)CCC3)n2)cn1]

4-chloro-6-(1-methylpyrazol-4-yl)pyrazolo[1,5-a]pyrazine (238 mg, 1.02 mmol), benzyl 5-(4,4,5,5-tetramethyl-1,3,2-dioxaborolan-2-yl)-3,4-dihydro-2H-pyridine-1-carboxylate (524.41 mg, 1.53 mmol), K₂CO₃ (422.34 mg, 3.06 mmol), tetrakis(triphenylphosphine)palladium(0) (235.41 mg, 203.72 umol) in water (1 mL) and dioxane (5 mL) was degassed and heated to 95° C. for 16 h. After cooling to rt, the mix was diluted with EtOAc and filtrated through celite. The concentrated residue was purified by silica gel column chromatography (DCM/EtOAc 0-100%) to give benzyl 5-[6-(1-methylpyrazol-4-yl)pyrazolo[1,5-a]pyrazin-4-yl]-3,4-dihydro-2H-pyridine-1-carboxylate (363 mg, 875.84 umol, 85.99% yield) as a white solid. LCMS (ESI+): m/z calcd. for C₂₃H₂₃N₆O₂ [M+H]⁺, 415.2; found, 415.1. Rt=0.84 min.

### 2. Preparation of 6-(1-methylpyrazol-4-yl)-4-(3-piperidyl)pyrazolo[1,5-a]pyrazine

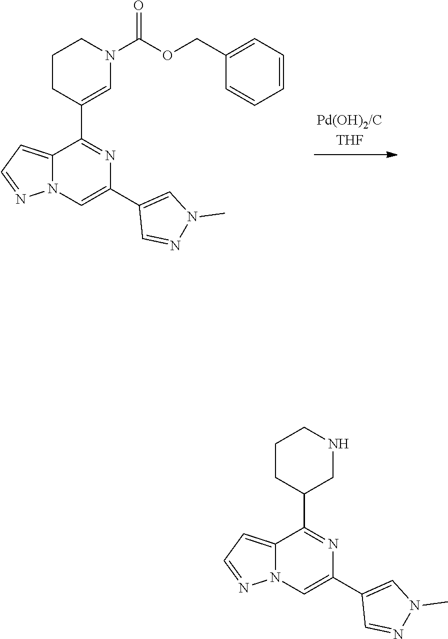 [SMILES: Cn1cc(-c2cn3nccc3c(C3=CN(C(=O)OCc4ccccc4)CCC3)n2)cn1 ; Cn1cc(-c2cn3nccc3c(C3CCCNC3)n2)cn1]

Benzyl 5-[6-(1-methylpyrazol-4-yl)pyrazolo[1,5-a]pyrazin-4-yl]-3,4-dihydro-2H-pyridine-1-carboxylate (363 mg, 875.84 umol) in THF (10 mL) was added Pd(OH)₂/C (123.00 mg, 875.84 umol) and stirred under H₂ for 2 days. After filtration through celite, the concentrated residue was used as is for the next step. LCMS (ESI+): m/z calcd. for C₁₅H₁₉N₆ [M+H]⁺, 283.2; found, 283.0. Rt=0.49 min.

### 3. Preparation of 1-[3-[6-(1-methylpyrazol-4-yl)pyrazolo[1,5-a]pyrazin-4-yl]-1-piperidyl]prop-2-en-1-one

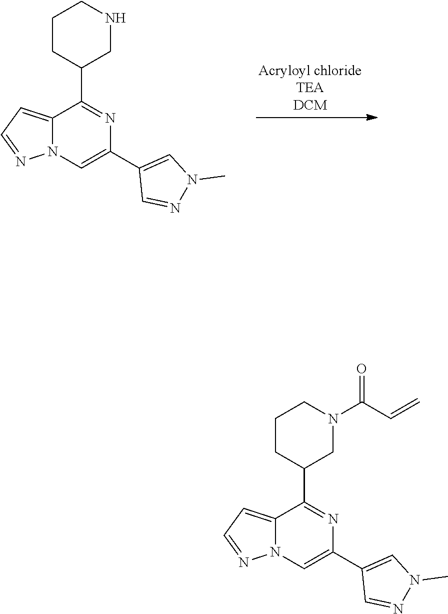 [SMILES: Cn1cc(-c2cn3nccc3c(C3CCCNC3)n2)cn1 ; C=CC(=O)N1CCCC(c2nc(-c3cnn(C)c3)cn3nccc23)C1]

6-(1-methylpyrazol-4-yl)-4-(3-piperidyl)pyrazolo[1,5-a]pyrazine (126 mg, 446.27 umol) in DCM (4 mL) was added TEA (90.32 mg, 892.53 umol, 124.40 uL) and stirred for 5 min. After cooling to 0° C., acryloyl chloride (48.47 mg, 535.52 umol, 43.51 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-15%) to give 1-[3-[6-(1-methylpyrazol-4-yl)pyrazolo[1,5-a]pyrazin-4-yl]-1-piperidyl]prop-2-en-1-one (18.3 mg, 53.31 umol, 12% yield, 98% purity) as a white solid. LCMS (ESI+): m/z calcd. for C₁₈H₂₁N₆O [M+H]⁺, 337.2; found, 337.1. Rt=0.58 min. ¹H NMR (400 MHz, CDCl₃, d): 8.49-8.43 (m, 1H), 8.01-7.89 (m, 3H), 6.95-6.79 (m, 1H), 6.72-6.59 (m, 1H), 6.39-6.27 (m, 1H), 5.79-5.66 (m, 1H), 5.03-4.83 (m, 1H), 4.74-4.58 (m, 1H), 4.03 (s, 3H), 3.38-3.17 (m, 2H), 2.34-2.20 (m, 2H), 2.16-2.06 (m, 1H), 2.00-1.93 (m, 1H), 1.80-1.68 (m, 1H).

### Example 6. 1-[4-[6-(1-methylpyrazol-4-yl)pyrazolo[1,5-a]pyrazin-4-yl]azepan-1-yl]prop-2-en-1-one

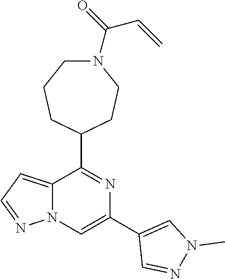 [SMILES: C=CC(=O)N1CCCC(c2nc(-c3cnn(C)c3)cn3nccc23)CC1]

### 1. Preparation of tert-butyl 4-oxoazepane-1-carboxylate

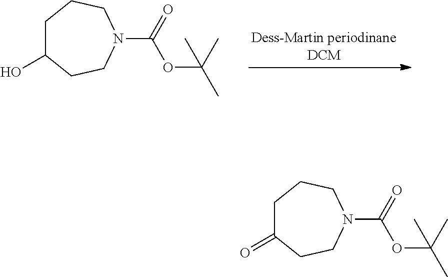

Tert-butyl 4-hydroxyazepane-1-carboxylate (300 mg, 1.39 mmol) in DCM (10 mL) was added Dess-Martin periodinane (886.55 mg, 2.09 mmol) and stirred at rt for 16 h. After filtration through celite, the concentrated residue was purified by silica gel column chromatography (HE/EA 0-100%) to give tert-butyl 4-oxoazepane-1-carboxylate (297 mg, 1.39 mmol, 99.94% yield) as an oil. ¹H NMR (400 MHz, CDCl₃, d): 3.50-3.66 (m, 4H), 2.55-2.73 (m, 4H), 1.73-1.86 (m, 2H), 1.46 (s, 9H).

### 2. Preparation of tert-butyl 5-(trifluoromethylsulfonyloxy)-2,3,4,7-tetrahydroazepine-1-carboxylate

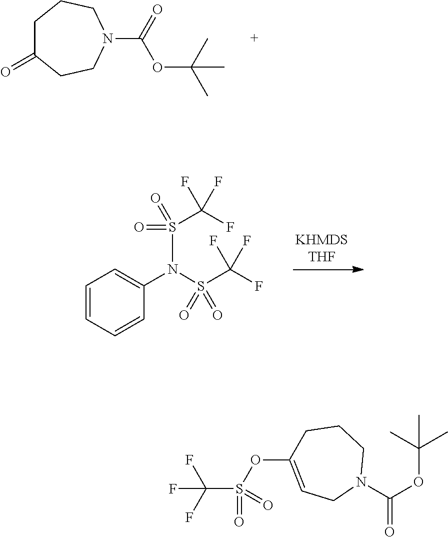 [SMILES: CC(C)(C)OC(=O)N1CCCC(=O)CC1 ; O=S(=O)(N(c1ccccc1)S(=O)(=O)C(F)(F)F)C(F)(F)F ; CC(C)(C)OC(=O)N1CC=C(OS(=O)(=O)C(F)(F)F)CCC1]

Trt-butyl 4-oxoazepane-1-carboxylate (297 mg, 1.39 mmol) was cooled to −78° C. and [bis(trimethylsilyl)amino]potassium (1 M, 1.48 mL) was added dropwise. After stirred at 0° C. for 2 h, 1,1,1-trifluoro-N-phenyl-N-(trifluoromethylsulfonyl)methanesulfonamide (746.25 mg, 2.09 mmol) was added in one pot. The mixture was allowed to warm to rt overnight. NH₄Cl (sat'd) was added and extracted with EA. The organic layers were dried over sodium sulfate, filtered and concentrated. The residue was purified by silica gel column chromatography (EA/Hept. (0-100%) to give tert-butyl 5-(trifluoromethylsulfonyloxy)-2,3,4,7-tetrahydroazepine-1-carboxylate (404 mg, 1.17 mmol, 84% yield) as a colorless oil.

### 3. Preparation of tert-butyl 5-(4,4,5,5-tetramethyl-1,3,2-dioxaborolan-2-yl)-2,3,4,7-tetrahydroazepine-1-carboxylate

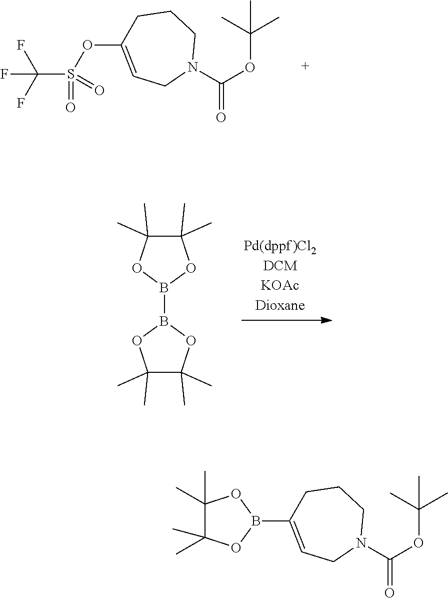

To a solution of tert-butyl 5-(trifluoromethylsulfonyloxy)-2,3,4,7-tetrahydroazepine-1-carboxylate (404 mg, 1.17 mmol) and 4,4,5,5-tetramethyl-2-(4,4,5,5-tetramethyl-1,3,2-dioxaborolan-2-yl)-1,3,2-dioxaborolane (594.15 mg, 2.34 mmol) in dioxane (5 mL) was added KOAc (344.44 mg, 3.51 mmol) and Pd(dppf)Cl₂ DCM (95.54 mg, 116.99 umol). The mixture was stirred at 90° C. under N₂ for 12 hours. After filtration through celite, the reaction mixture was concentrated under vacuum to give a crude, which was purified by silica gel column chromatography (PE/EA=7/3) to give tert-butyl 5-(4,4,5,5-tetramethyl-1,3,2-dioxaborolan-2-yl)-2,3,4,7-tetrahydroazepine-1-carboxylate (550 mg) as white solid.

### 4. Preparation of tert-butyl 5-[6-(1-methylpyrazol-4-yl)pyrazolo[1,5-a]pyrazin-4-yl]-2,3,4,7-tetrahydroazepine-1-carboxylate

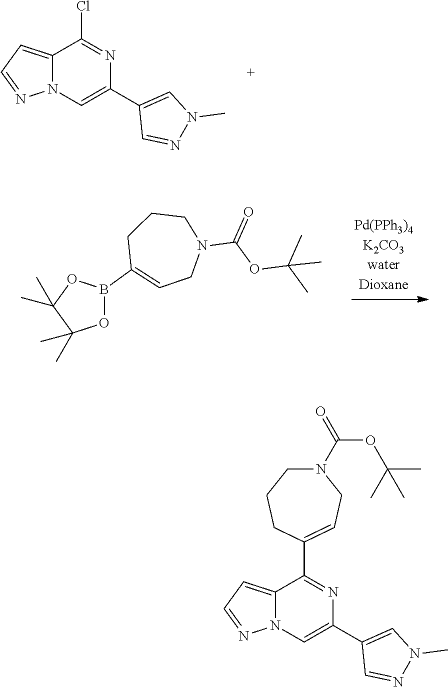 [SMILES: Cn1cc(-c2cn3nccc3c(Cl)n2)cn1 ; CC(C)(C)OC(=O)N1CC=C(B2OC(C)(C)C(C)(C)O2)CCC1 ; Cn1cc(-c2cn3nccc3c(C3=CCN(C(=O)OC(C)(C)C)CCC3)n2)cn1]

4-chloro-6-(1-methylpyrazol-4-yl)pyrazolo[1,5-a]pyrazine (263.45 mg, 1.13 mmol), tert-butyl 4-(4,4,5,5-tetramethyl-1,3,2-dioxaborolan-2-yl)cyclohept-3-ene-1-carboxylate (545 mg, 1.69 mmol), K₂CO₃ (467.50 mg, 3.38 mmol), tetrakis(triphenylphosphine)palladium(0) (260.58 mg, 225.50 umol) in water (1 mL) and dioxane (5 mL) was degassed and heated to 95° C. for 16 h. After cooling to rt, the mix was diluted with EtOAc and filtrated through celite. The concentrated residue was purified by silica gel column chromatography (DCM/EtOAc 0-100%) to give tert-butyl 5-[6-(1-methylpyrazol-4-yl)pyrazolo[1,5-a]pyrazin-4-yl]-2,3,4,7-tetrahydroazepine-1-carboxylate (404 mg, 1.02 mmol, 91% yield) as an oil. LCMS (ESI+): m/z calcd. for C₂₁H₂₇N₆O₂ [M+H]⁺, 395.2; found, 395.2. Rt=0.83 min.

### 5. Preparation of tert-butyl 4-[6-(1-methylpyrazol-4-yl)pyrazolo[1,5-a]pyrazin-4-yl]azepane-1-carboxylate

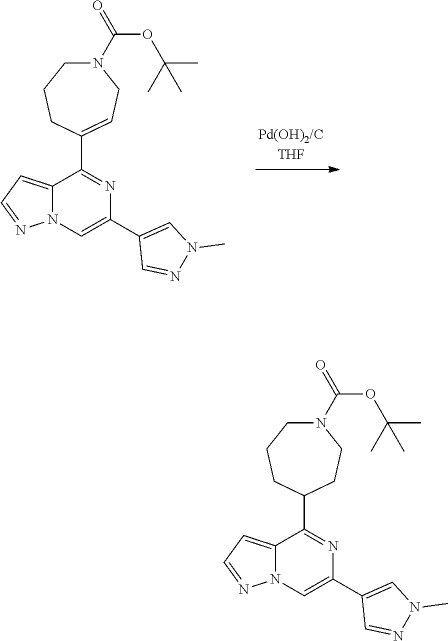 [SMILES: Cn1cc(-c2cn3nccc3c(C3=CCN(C(=O)OC(C)(C)C)CCC3)n2)cn1 ; Cn1cc(-c2cn3nccc3c(C3CCCN(C(=O)OC(C)(C)C)CC3)n2)cn1]

Pd(OH)₂/C (71.92 mg, 102.42 umol, 20% purity) in flask was added tert-butyl 5-[6-(1-methylpyrazol-4-yl)pyrazolo[1,5-a]pyrazin-4-yl]-2,3,4,7-tetrahydroazepine-1-carboxylate (404 mg, 1.02 mmol) in THF (5 mL) solution. The mix was stirred under 50 bar H₂ for 16 h. After filtration through celite, the concentrated residue tert-butyl 4-[6-(1-methylpyrazol-4-yl)pyrazolo[1,5-a]pyrazin-4-yl]azepane-1-carboxylate (315 mg, 794.48 umol, 78% yield) was used as is. LCMS (ESI+): m/z calcd. for C₂₁H₂₉N₆O₂ [M+H]⁺, 397.2; found, 397.3. Rt=0.86 min.

### 6. Preparation of 4-(azepan-4-yl)-6-(1-methylpyrazol-4-yl)pyrazolo[1,5-a]pyrazine

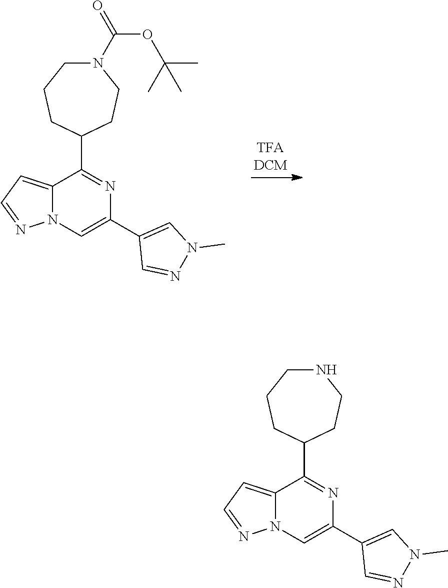 [SMILES: Cn1cc(-c2cn3nccc3c(C3CCCN(C(=O)OC(C)(C)C)CC3)n2)cn1 ; Cn1cc(-c2cn3nccc3c(C3CCCNCC3)n2)cn1]

Tert-butyl 4-[6-(1-methylpyrazol-4-yl)pyrazolo[1,5-a]pyrazin-4-yl]azepane-1-carboxylate (315 mg, 794.48 umol) in DCM (3 mL) was added TFA (744.50 mg, 6.53 mmol, 0.5 mL) and stirred at for 16 h. After concentration, the crude residue 4-(azepan-4-yl)-6-(1-methylpyrazol-4-yl)pyrazolo[1,5-a]pyrazine (342 mg, TFA salt) was used as is for the next step. LCMS (ESI+): m/z calcd. for C₁₆H₂₁N₆[M+H]⁺, 297.2; found, 297.1. Rt=0.48 min.

### 7. Preparation of 1-[4-[6-(1-methylpyrazol-4-yl)pyrazolo[1,5-a]pyrazin-4-yl]azepan-1-yl]prop-2-en-1-one

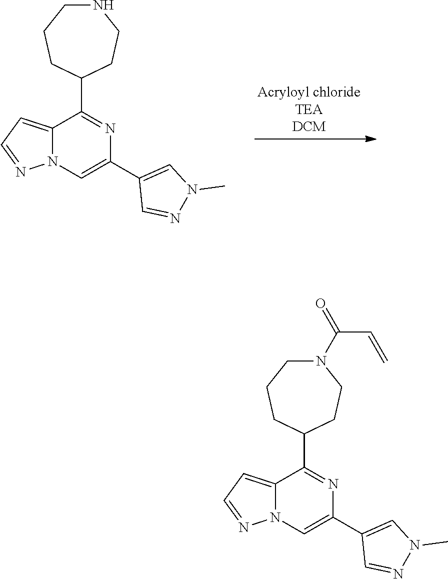 [SMILES: Cn1cc(-c2cn3nccc3c(C3CCCNCC3)n2)cn1 ; C=CC(=O)N1CCCC(c2nc(-c3cnn(C)c3)cn3nccc23)CC1]

4-(azepan-4-yl)-6-(1-methylpyrazol-4-yl)pyrazolo[1,5-a]pyrazine (342 mg, 833.35 umol, TFA salt) in DCM (4 mL) was added TEA (168.65 mg, 1.67 mmol, 232.30 uL) and stirred for 5 min. After cooling to 0° C., acryloyl chloride (90.51 mg, 1.00 mmol, 81.25 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-15%) then prep HPLC to give 1-[4-[6-(1-methylpyrazol-4-yl)pyrazolo[1,5-a]pyrazin-4-yl]azepan-1-yl]prop-2-en-1-one (17.2 mg, 46.63 umol, 5.6% yield, 95% purity) as a white solid. LCMS (ESI+): m/z calcd. for C₁₉H₂₃N₆O [M+H]⁺, 351.1; found, 351.1. Rt=0.58 min. ¹H NMR (400 MHz CDCl₃, d): 8.47-8.54 (m, 1H), 8.26-8.34 (m, 1H), 8.04 (s, 1H), 7.88-7.92 (m, 1H), 6.79-6.92 (m, 1H), 6.62-6.70 (m, 1H), 6.41-6.47 (m, 1H), 5.72-5.78 (m, 1H), 3.99-4.03 (m, 3H), 3.63-3.86 (m, 4H), 1.82-2.33 (m, 7H).

### Example 7. 1-[4-[3-fluoro-6-(1-methylpyrazol-4-yl)pyrazolo[1,5-a]pyrazin-4-yl]-1-piperidyl]prop-2-en-1-one

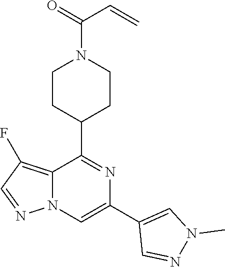 [SMILES: C=CC(=O)N1CCC(c2nc(-c3cnn(C)c3)cn3ncc(F)c23)CC1]

### 1. Preparation of tert-butyl 4-(6-chloro-3-fluoro-pyrazolo[1,5-a]pyrazin-4-yl)-3,6-dihydro-2H-pyridine-1-carboxylate

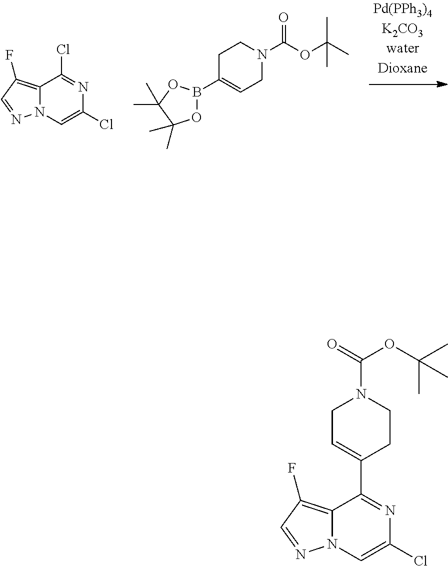 [SMILES: Fc1cnn2cc(Cl)nc(Cl)c12 ; CC(C)(C)OC(=O)N1CC=C(B2OC(C)(C)C(C)(C)O2)CC1 ; CC(C)(C)OC(=O)N1CC=C(c2nc(Cl)cn3ncc(F)c23)CC1]

4,6-dichloro-3-fluoro-pyrazolo[1,5-a]pyrazine (200 mg, 970.85 umol), tert-butyl 4-(4,4,5,5-tetramethyl-1,3,2-dioxaborolan-2-yl)-3,6-dihydro-2H-pyridine-1-carboxylate (300.20 mg, 970.85 umol), K₂CO₃ (402.54 mg, 2.91 mmol), tetrakis(triphenylphosphine)palladium(0) (112.19 mg, 97.09 umol) in water (0.3 mL) and dioxane (2 mL) was degassed and heated to 95° C. for 16 h. After cooling to rt, the mix was diluted with EtOAc and filtrated through celite. The concentrated residue was purified by silica gel column chromatography (DCM/EtOAc 0-100%) to give tert-butyl 4-(6-chloro-3-fluoro-pyrazolo[1,5-a]pyrazin-4-yl)-3,6-dihydro-2H-pyridine-1-carboxylate (246 mg, 697.30 umol, 72% yield) as a white solid. LCMS (ESI+): m/z calcd. for C₁₁H₁₁ClFN₄ [M+H-Boc]⁺, 253.1; found, 253.0. Rt=0.96 min.

### 2. Preparation of tert-butyl 4-[3-fluoro-6-(1-methylpyrazol-4-yl)pyrazolo[1,5-a]pyrazin-4-yl]-3,6-dihydro-2H-pyridine-1-carboxylate

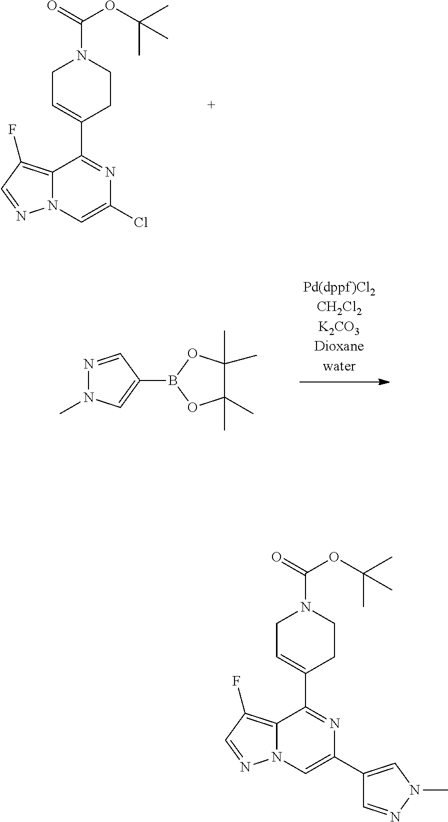

Tert-butyl 4-(6-chloro-3-fluoro-pyrazolo[1,5-a]pyrazin-4-yl)-3,6-dihydro-2H-pyridine-1-carboxylate (246 mg, 697.30 umol), 1-methyl-4-(4,4,5,5-tetramethyl-1,3,2-dioxaborolan-2-yl)pyrazole (217.62 mg, 1.05 mmol), Pd(dppf)Cl₂ CH₂Cl₂ (56.94 mg, 69.73 umol), K₂CO₃ (289.12 mg, 2.09 mmol) in dioxane (2 mL) and water (0.3 mL) was degassed and heated to 95° C. for 16 h. After cooling to rt, the mix was filtrated though celite and concentrated. The residue was purified by silica gel column chromatography (HE/EA 0-100%) to give tert-butyl 4-[3-fluoro-6-(1-methylpyrazol-4-yl)pyrazolo[1,5-a]pyrazin-4-yl]-3,6-dihydro-2H-pyridine-1-carboxylate (236 mg, 592.32 umol, 85% yield) as a light-yellow gel. LCMS (ESI+): m/z calcd. for C₂₀H₂₄FN₆O₂[M+H]⁺, 399.2; found, 399.2. Rt=0.86 min.

### 3. Preparation of 3-fluoro-6-(1-methylpyrazol-4-yl)-4-(1,2,3,6-tetrahydropyridin-4-yl)pyrazolo[1,5-a]pyrazine

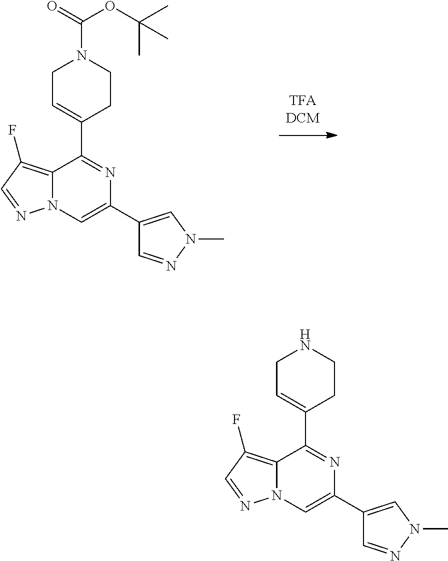 [SMILES: Cn1cc(-c2cn3ncc(F)c3c(C3=CCN(C(=O)OC(C)(C)C)CC3)n2)cn1 ; Cn1cc(-c2cn3ncc(F)c3c(C3=CCNCC3)n2)cn1]

Tert-butyl 4-[3-fluoro-6-(1-methylpyrazol-4-yl)pyrazolo[1,5-a]pyrazin-4-yl]-3,6-dihydro-2H-pyridine-1-carboxylate (236 mg, 592.32 umol) in DCM (3 mL) was added TFA (744.50 mg, 6.53 mmol, 0.5 mL) and stirred at for 16 h. After concentration, the crude residue was used as is for the next step. LCMS (ESI+): m/z calcd. for C₁₅H₁₆FN₆ [M+H]⁺, 299.2; found, 299.0. Rt=0.53 min.

### 4. Preparation of 3-fluoro-6-(1-methylpyrazol-4-yl)-4-(4-piperidyl)pyrazolo[1,5-a]pyrazine

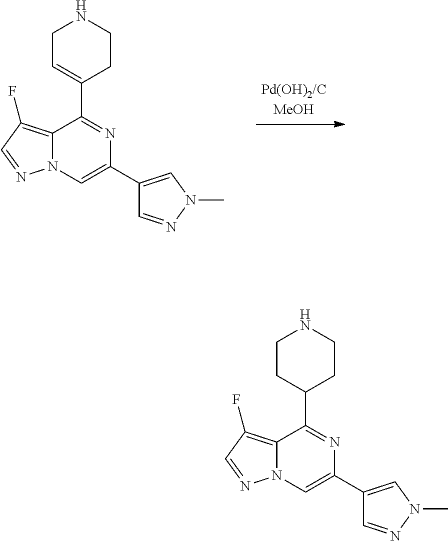 [SMILES: Cn1cc(-c2cn3ncc(F)c3c(C3=CCNCC3)n2)cn1 ; Cn1cc(-c2cn3ncc(F)c3c(C3CCNCC3)n2)cn1]

Pd(OH)₂/C (187.33 mg, 266.77 umol, 20% purity) in flask was added 3-fluoro-6-(1-methylpyrazol-4-yl)-4-(1,2,3,6-tetrahydropyridin-4-yl)pyrazolo[1,5-a]pyrazine (550 mg, 1.33 mmol, TFA) in MeOH (5 mL) solution. The mix was stirred under H₂ for 16 h. After filtration and concentration, the crude was used as is for the next step. LCMS (ESI+): m/z calcd. for C₁₅H₁₈FN₆ [M+H]⁺, 301.2; found, 301.1. Rt=0.48 min.

### 5. Preparation of 1-[4-[3-fluoro-6-(1-methylpyrazol-4-yl)pyrazolo[1,5-a]pyrazin-4-yl]-1-piperidyl]prop-2-en-1-one

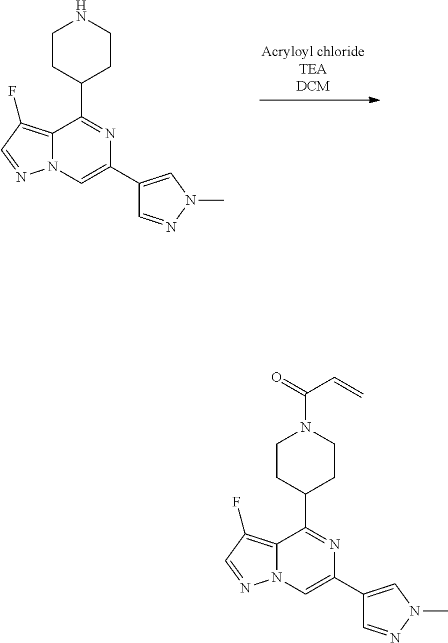 [SMILES: Cn1cc(-c2cn3ncc(F)c3c(C3CCNCC3)n2)cn1 ; C=CC(=O)N1CCC(c2nc(-c3cnn(C)c3)cn3ncc(F)c23)CC1]

3-fluoro-6-(1-methylpyrazol-4-yl)-4-(4-piperidyl)pyrazolo[1,5-a]pyrazine (233 mg, 775.80 umol, TFA salt) in DCM (5 mL) was added TEA (157.01 mg, 1.55 mmol, 216.26 uL) and stirred for 5 min. After cooling to 0° C., acryloyl chloride (84.26 mg, 930.97 umol, 75.64 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-15%) then prep HPLC to give 1-[4-[3-fluoro-6-(1-methylpyrazol-4-yl)pyrazolo[1,5-a]pyrazin-4-yl]-1-piperidyl]prop-2-en-1-one (36 mg, 96.51 umol, 12% yield, 95% purity) as a white solid. LCMS (ESI+): m/z calcd. for C₁₈FH₂₀N₆O [M+H]⁺, 355.2; found, 355.1. Rt=0.63 min. ¹H NMR (400 MHz, CDCl₃, d): 8.09-8.36 (m, 2H), 7.91-8.02 (m, 1H), 7.79-7.88 (m, 1H), 6.60-6.74 (m, 1H), 6.29-6.41 (m, 1H), 5.69-5.79 (m, 1H), 3.88-4.14 (m, 3H), 3.48-3.62 (m, 1H), 3.02-3.29 (m, 2H), 1.87-2.16 (m, 6H). ¹⁹F NMR (376 MHz, CDCl₃, d): −171.56 (s, 1F).

### Example 8. 1-[4-[6-(1-methylpyrazol-4-yl)pyrazolo[1,5-a]pyridin-4-yl]-1-piperidyl]prop-2-en-1-one

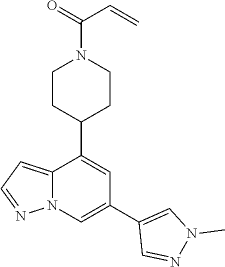 [SMILES: C=CC(=O)N1CCC(c2cc(-c3cnn(C)c3)cn3nccc23)CC1]

### 1. Preparation of[6-(1-methylpyrazol-4-yl)pyrazolo[1,5-a]pyridin-4-yl]trifluoromethanesulfonate

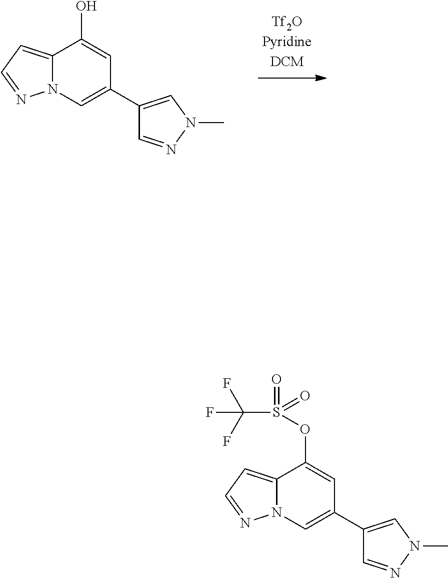 [SMILES: Cn1cc(-c2cc(O)c3ccnn3c2)cn1 ; Cn1cc(-c2cc(OS(=O)(=O)C(F)(F)F)c3ccnn3c2)cn1]

6-(1-methylpyrazol-4-yl)pyrazolo[1,5-a]pyridin-4-ol (200 mg, 933.61 umol) in DCM (4 mL) was added pyridine (147.70 mg, 1.87 mmol, 151.02 uL) and triflic anhydride (316.09 mg, 1.12 mmol, 188.48 uL) and stirred at rt for 16 h. After concentration, the residue was purified by silica gel column chromatography (EA/HE 0-100%) to give [6-(1-methylpyrazol-4-yl)pyrazolo[1,5-a]pyridin-4-yl]trifluoromethanesulfonate (219 mg, 632.43 umol, 68% yield) as a white solid. LCMS (ESI+): m/z calcd. for Cl₂H₁₀F₃N₄O₃S [M+H]⁺, 347.0; found, 347.0. Rt=0.79 min.

### 2. Preparation of tert-butyl 4-[6-(1-methylpyrazol-4-yl)pyrazolo[1,5-a]pyridin-4-yl]-3,6-dihydro-2H-pyridine-1-carboxylate

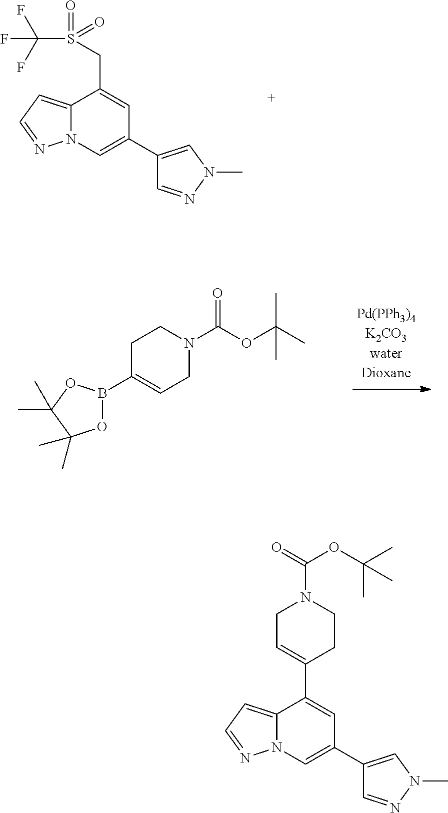 [SMILES: Cn1cc(-c2cc(CS(=O)(=O)C(F)(F)F)c3ccnn3c2)cn1 ; CC(C)(C)OC(=O)N1CC=C(B2OC(C)(C)C(C)(C)O2)CC1 ; Cn1cc(-c2cc(C3=CCN(C(=O)OC(C)(C)C)CC3)c3ccnn3c2)cn1]

[6-(1-methylpyrazol-4-yl)pyrazolo[1,5-a]pyridin-4-yl]trifluoromethanesulfonate (219 mg, 632.43 umol), tert-butyl 4-(4,4,5,5-tetramethyl-1,3,2-dioxaborolan-2-yl)-3,6-dihydro-2H-pyridine-1-carboxylate (195.55 mg, 632.43 umol), K₂CO₃ (262.22 mg, 1.90 mmol), tetrakis(triphenylphosphine)palladium(0) (73.08 mg, 63.24 umol) in water (0.3 mL) and dioxane (2 mL) was degassed and heated to 95° C. for 16 h. After cooling to rt, the mix was diluted with EtOAc and filtrated through celite. The concentrated residue was purified by silica gel column chromatography (EtOAc/DCM 0-100%) to give tert-butyl 4-[6-(1-methylpyrazol-4-yl)pyrazolo[1,5-a]pyridin-4-yl]-3,6-dihydro-2H-pyridine-1-carboxylate (235 mg, 619.31 umol, 98% yield) as a gel. LCMS (ESI+): m/z calcd. for C₂₁H₂₆N₅O₂ [M+H]⁺, 380.2; found, 380.2. Rt=0.82 min.

### 3. Preparation of 6-(1-methylpyrazol-4-yl)-4-(1,2,3,6-tetrahydropyridin-4-yl)pyrazolo[1,5-a]pyridine

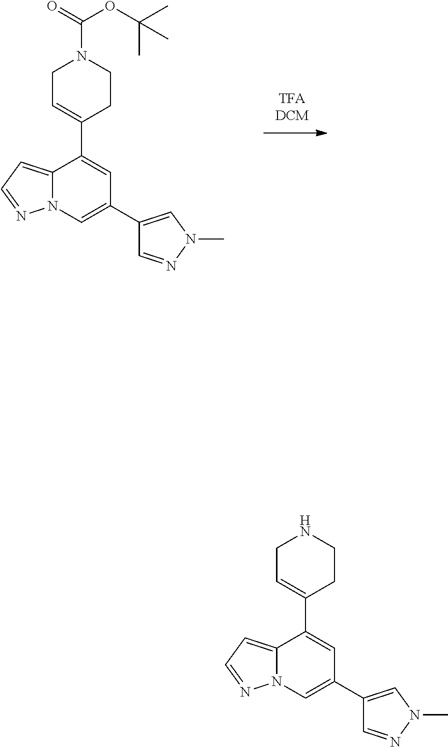 [SMILES: Cn1cc(-c2cc(C3=CCN(C(=O)OC(C)(C)C)CC3)c3ccnn3c2)cn1 ; Cn1cc(-c2cc(C3=CCNCC3)c3ccnn3c2)cn1]

Tert-butyl 4-[6-(1-methylpyrazol-4-yl)pyrazolo[1,5-a]pyridin-4-yl]-3,6-dihydro-2H-pyridine-1-carboxylate (235 mg, 619.31 umol) in DCM (3 mL) was added TFA (744.50 mg, 6.53 mmol, 0.5 mL) and stirred at rt for 1 h. The crude was concentrated and used as is for the next step. LCMS (ESI+): m/z calcd. for C₁₆H₁₈N₅[M+H]⁺, 280.2; found, 280.0. Rt=0.50 min.

### 4. Preparation of 6-(1-methylpyrazol-4-yl)-4-(4-piperidyl)pyrazolo[1,5-a]pyridine

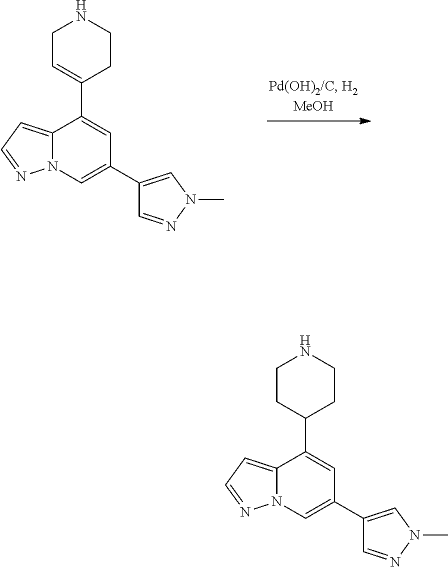 [SMILES: Cn1cc(-c2cc(C3=CCNCC3)c3ccnn3c2)cn1 ; Cn1cc(-c2cc(C3CCNCC3)c3ccnn3c2)cn1]

6-(1-methylpyrazol-4-yl)-4-(1,2,3,6-tetrahydropyridin-4-yl)pyrazolo[1,5-a]pyridine (657 mg, 2.35 mmol) in MeOH (6 mL) was added Pd(OH)₂/C (165.16 mg, 235.20 umol, 20% purity) and stirred under H₂ at rt for 16 h. After filtration through celite, the concentrated residue was used as for the next step. LCMS (ESI+): m/z calcd. for C₁₆H₂₀N₅[M+H]⁺, 282.2; found, 282.1. Rt=0.48 min.

### 5. Preparation of 1-[4-[6-(1-methylpyrazol-4-yl)pyrazolo[1,5-a]pyridin-4-yl]-1-piperidyl]prop-2-en-1-one

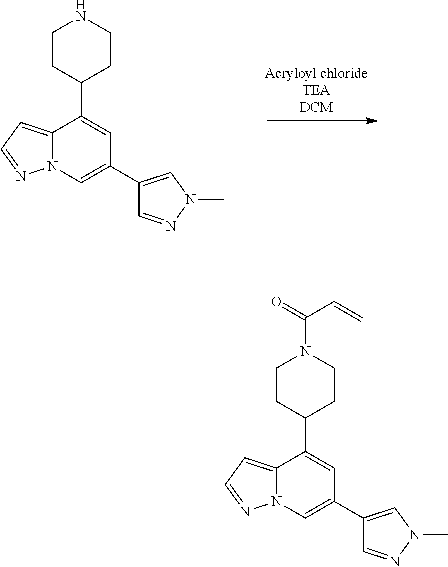 [SMILES: Cn1cc(-c2cc(C3CCNCC3)c3ccnn3c2)cn1 ; C=CC(=O)N1CCC(c2cc(-c3cnn(C)c3)cn3nccc23)CC1]

6-(1-methylpyrazol-4-yl)-4-(4-piperidyl)pyrazolo[1,5-a]pyridine (249 mg, 629.78 umol, TFA salt) in DCM (5 mL) was added TEA (127.45 mg, 1.26 mmol, 175.56 uL) and stirred for 5 min. After cooling to 0° C., acryloyl chloride (68.40 mg, 755.73 umol, 61.40 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-15%) then prep HPLC to give 1-[4-[6-(1-methylpyrazol-4-yl)pyrazolo[1,5-a]pyridin-4-yl]-1-piperidyl]prop-2-en-1-one (82 mg, 244.48 umol, 39% yield) as a white solid. LCMS (ESI+): m/z calcd. for C₁₉H₂₂N₅₀ [M+H]⁺, 336.2; found, 336.1. Rt=0.57 min. ¹H NMR (400 MHz, CDCl₃, d): 8.56 (s, 1H), 7.95 (d, J=2.3 Hz, 1H), 7.75 (d, J=0.8 Hz, 1H), 7.55-7.69 (m, 1H), 7.05 (s, 1H), 6.59-6.74 (m, 1H), 6.56 (dd, J=0.9, 2.38 Hz, 1H), 6.34 (dd, J=2.0, 16.82 Hz, 1H), 5.74 (dd, J=1.8, 10.5 Hz, 1H), 4.86-5.03 (m, 1H), 4.13-4.31 (m, 1H), 3.98 (s, 3H), 3.22-3.37 (m, 1H), 2.97-3.22 (m, 1H), 2.82 (br s, 1H), 2.07-2.16 (m, 2H), 1.80 (br s, 2H).

### Example 9. 1-[4-[3-methyl-6-(1-methylpyrazol-4-yl)pyrazolo[1,5-a]pyrazin-4-yl]-1-piperidyl]prop-2-en-1-one

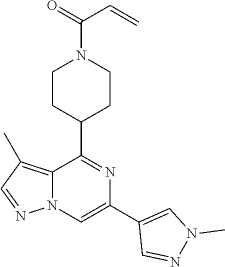 [SMILES: C=CC(=O)N1CCC(c2nc(-c3cnn(C)c3)cn3ncc(C)c23)CC1]

### 1. Preparation of tert-butyl 4-(6-chloro-3-methyl-pyrazolo[1,5-a]pyrazin-4-yl)-3,6-dihydro-2H-pyridine-1-carboxylate

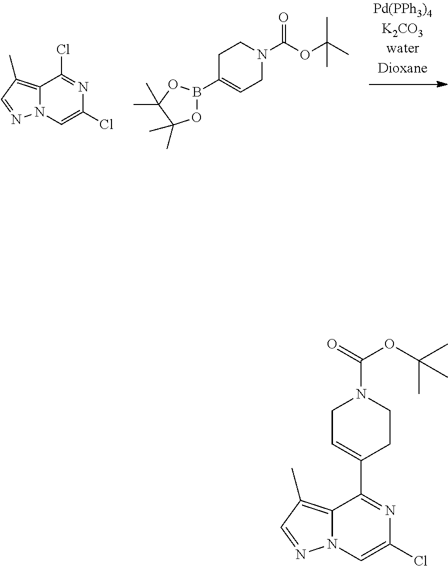 [SMILES: Cc1cnn2cc(Cl)nc(Cl)c12 ; CC(C)(C)OC(=O)N1CC=C(B2OC(C)(C)C(C)(C)O2)CC1 ; Cc1cnn2cc(Cl)nc(C3=CCN(C(=O)OC(C)(C)C)CC3)c12]

4,6-dichloro-3-methyl-pyrazolo[1,5-a]pyrazine (200 mg, 989.90 umol), tert-butyl 4-(4,4,5,5-tetramethyl-1,3,2-dioxaborolan-2-yl)-3,6-dihydro-2H-pyridine-1-carboxylate (306.09 mg, 989.90 umol), K₂CO₃ (410.44 mg, 2.97 mmol), tetrakis(triphenylphosphine)palladium(0) (114.39 mg, 98.99 umol) in water (0.3 mL) and dioxane (2 mL) was degassed and heated to 95° C. for 16 h. After cooling to rt, the mix was diluted with EtOAc and filtrated through celite. The concentrated residue was purified by silica gel column chromatography (EtOAc/DCM 0-100%) to give tert-butyl 4-(6-chloro-3-methyl-pyrazolo[1,5-a]pyrazin-4-yl)-3,6-dihydro-2H-pyridine-1-carboxylate (281 mg, 805.56 umol, 82% yield) as a gel. LCMS (ESI+): m/z calcd. for C₁₇H₂₂C₁N₄O₂[M+H]⁺, 349.1; found, 349.0. Rt=0.94 min.

### 2. Preparation of tert-butyl 4-[3-methyl-6-(1-methylpyrazol-4-yl)pyrazolo[1,5-a]pyrazin-4-yl]-3,6-dihydro-2H-pyridine-1-carboxylate

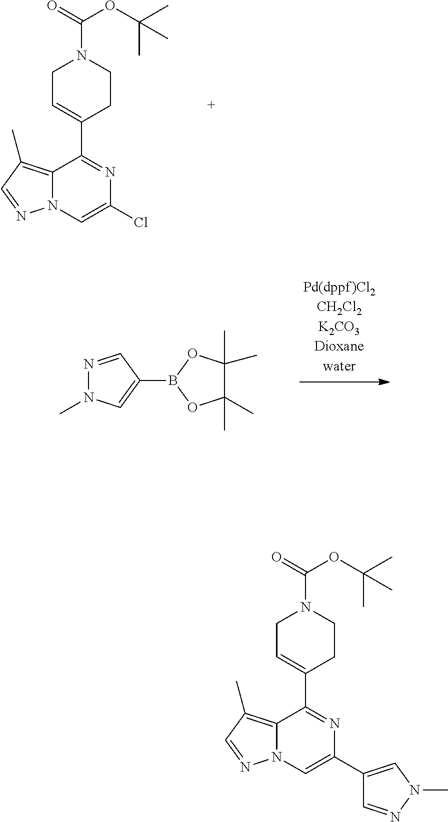

Tert-butyl 4-(6-chloro-3-methyl-pyrazolo[1,5-a]pyrazin-4-yl)-3,6-dihydro-2H-pyridine-1-carboxylate (281 mg, 805.56 umol), 1-methyl-4-(4,4,5,5-tetramethyl-1,3,2-dioxaborolan-2-yl)pyrazole (251.41 mg, 1.21 mmol), Pd(dppf)Cl₂ CH₂Cl₂ (65.79 mg, 80.56 umol), K₂CO₃ (334.01 mg, 2.42 mmol) in dioxane (2 mL) and water (0.3 mL) was degassed and heated to 95° C. for 16 h. After cooling to rt, the mix was filtrated though celite and concentrated. The residue was purified by silica gel column chromatography (HE/EA 0-100%) to give tert-butyl 4-[3-methyl-6-(1-methylpyrazol-4-yl)pyrazolo[1,5-a]pyrazin-4-yl]-3,6-dihydro-2H-pyridine-1-carboxylate (325 mg, 823.89 umol, 102% yield) as a light-yellow gel. LCMS (ESI+): m/z calcd. for C₂₁H₂₇N₆O₂ [M+H]⁺, 395.2; found, 395.3. Rt=0.85 min.

### 3. Preparation of 3-methyl-6-(1-methylpyrazol-4-yl)-4-(1,2,3,6-tetrahydropyridin-4-yl)pyrazolo[1,5-a]pyrazine

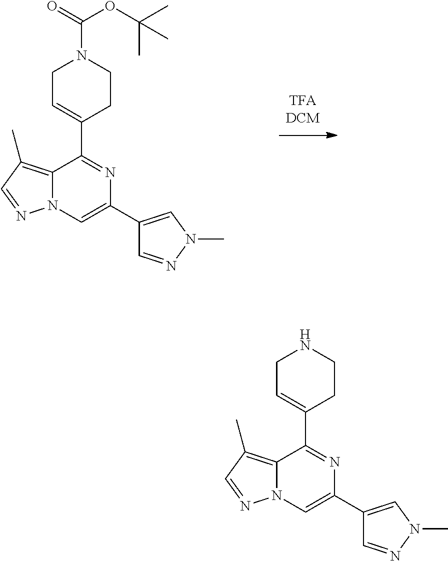 [SMILES: Cc1cnn2cc(-c3cnn(C)c3)nc(C3=CCN(C(=O)OC(C)(C)C)CC3)c12 ; Cc1cnn2cc(-c3cnn(C)c3)nc(C3=CCNCC3)c12]

Tert-butyl 4-[3-methyl-6-(1-methylpyrazol-4-yl)pyrazolo[1,5-a]pyrazin-4-yl]-3,6-dihydro-2H-pyridine-1-carboxylate (325 mg, 823.89 umol) in DCM (2 mL) was added TFA (744.50 mg, 6.53 mmol, 0.5 mL) and stirred at for 16 h. After concentration, the crude residue was used as is for the next step. LCMS (ESI+): m/z calcd. for C₁₆H₁₉N₆ [M+H]⁺, 295.2; found, 295.1. Rt=0.49 min.

### 4. Preparation of 3-methyl-6-(1-methylpyrazol-4-yl)-4-(4-piperidyl)pyrazolo[1,5-a]pyrazine

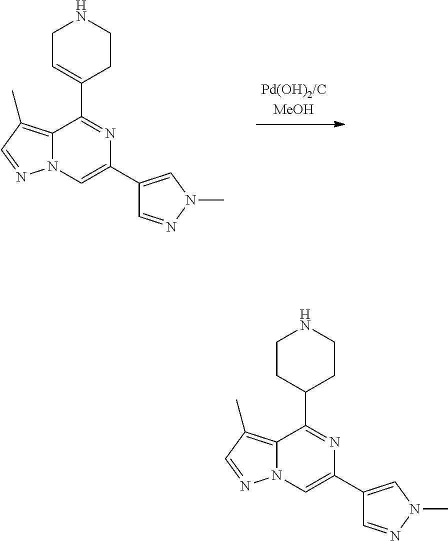 [SMILES: Cc1cnn2cc(-c3cnn(C)c3)nc(C3=CCNCC3)c12 ; Cc1cnn2cc(-c3cnn(C)c3)nc(C3CCNCC3)c12]

3-methyl-6-(1-methylpyrazol-4-yl)-4-(1,2,3,6-tetrahydropyridin-4-yl)pyrazolo[1,5-a]pyrazine (690 mg, 1.69 mmol, TFA salt) in MeOH (6 mL) was added Pd(OH)₂/C (118.64 mg, 168.96 umol, 20% purity) and stirred under H₂ at 60 psi for 16 h. After filtration through celite, the concentrated residue was used as is for the next step. LCMS (ESI+): m/z calcd. for C₁₆H₂₁N₆[M+H]⁺, 297.2; found, 297.2. Rt=0.41 min.

### 5. Preparation of 1-[4-[3-methyl-6-(1-methylpyrazol-4-yl)pyrazolo[1,5-a]pyrazin-4-yl]-1-piperidyl]prop-2-en-1-one

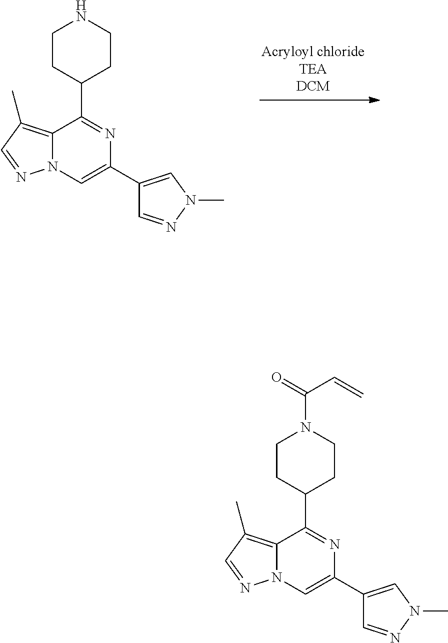 [SMILES: Cc1cnn2cc(-c3cnn(C)c3)nc(C3CCNCC3)c12 ; C=CC(=O)N1CCC(c2nc(-c3cnn(C)c3)cn3ncc(C)c23)CC1]

3-methyl-6-(1-methylpyrazol-4-yl)-4-(4-piperidyl)pyrazolo[1,5-a]pyrazine (226 mg, 762.56 umol) in DCM (4 mL) was added TEA (154.33 mg, 1.53 mmol, 212.57 uL) and stirred for 5 min. After cooling to 0° C., acryloyl chloride (82.82 mg, 915.08 umol, 74.35 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-15%) then prep HPLC to give 1-[4-[3-methyl-6-(1-methylpyrazol-4-yl)pyrazolo[1,5-a]pyrazin-4-yl]-1-piperidyl]prop-2-en-1-one (26.5 mg, 75.62 umol, 9.9% yield) as a white solid. LCMS (ESI+): m/z calcd. for C₁₉H₂₃N₆O [M+H]⁺, 351.2; found, 351.1. Rt=0.61 min. ¹H NMR (400 MHz, CDCl₃, d): 8.34-8.37 (m, 1H), 7.88-7.90 (m, 1H), 7.86-7.88 (m, 1H), 7.76-7.78 (m, 1H), 6.63-6.71 (m, 1H), 6.31-6.37 (m, 1H), 5.72-5.76 (m, 1H), 4.79-4.89 (m, 1H), 4.17-4.26 (m, 1H), 3.98-4.00 (m, 3H), 3.50-3.58 (m, 1H), 3.28-3.37 (m, 1H), 2.90-3.00 (m, 1H), 2.53-2.56 (m, 3H), 2.02-2.12 (m, 4H)

### Example 10. 1-[4-[3-methyl-6-(1-methylpyrazol-4-yl)pyrazolo[1,5-a]pyrazin-4-yl]-3,6-dihydro-2H-pyridin-1-yl]prop-2-en-1-one

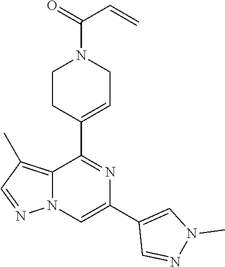 [SMILES: C=CC(=O)N1CC=C(c2nc(-c3cnn(C)c3)cn3ncc(C)c23)CC1]

1-[4-[3-methyl-6-(1-methylpyrazol-4-yl)pyrazolo[1,5-a]pyrazin-4-yl]-3,6-dihydro-2H-pyridin-1-yl]prop-2-en-1-one (20 mg, 54.53 umol, 95% purity) was isolated as a side product from Example 9 as a white solid. LCMS (ESI+): m/z calcd. for C₁₉H₂₁N₆O [M+H]⁺, 349.2; found, 349.2. Rt=0.57 min. ¹H NMR (400 MHz, CDCl₃, d): 8.41-8.47 (m, 1H), 7.97-8.05 (m, 1H), 7.87-7.92 (m, 1H), 7.81-7.84 (m, 1H), 6.62-6.76 (m, 1H), 6.33-6.43 (m, 1H), 6.01-6.15 (m, 1H), 5.75-5.83 (m, 1H), 4.35-4.50 (m, 2H), 3.88-4.09 (m, 5H), 2.77-2.92 (m, 2H), 2.29-2.37 (m, 3H).

### Example 11. 1-[3-[6-(1-methylpyrazol-4-yl)pyrazolo[1,5-a]pyrazin-4-yl]-8-azabicyclo [3.2.1]octan-8-yl]prop-2-en-1-one

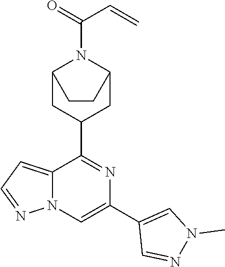 [SMILES: C=CC(=O)N1C2CCC1CC(c1nc(-c3cnn(C)c3)cn3nccc13)C2]

### 1. Preparation of tert-butyl 3-(trifluoromethylsulfonyloxy)-8-azabicyclo[3.2.1]oct-2-ene-8-carboxylate

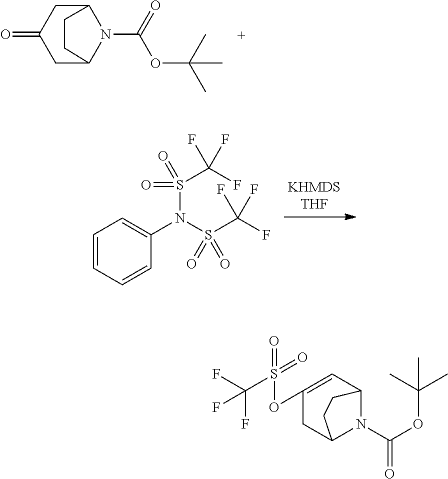 [SMILES: CC(C)(C)OC(=O)N1C2CCC1CC(=O)C2 ; O=S(=O)(N(c1ccccc1)S(=O)(=O)C(F)(F)F)C(F)(F)F ; CC(C)(C)OC(=O)N1C2C=C(OS(=O)(=O)C(F)(F)F)CC1CC2]

Tert-butyl 3-oxo-8-azabicyclo[3.2.1]octane-8-carboxylate (300 mg, 1.33 mmol) was cooled to −78° C. and [bis(trimethylsilyl)amino]potassium (1 M, 1.41 mL) was added dropwise. After stirred at 0° C. for 2 h, 1,1,1-trifluoro-N-phenyl-N-(trifluoromethylsulfonyl)methane sulfonamide (713.60 mg, 2.00 mmol) was added in one pot. The mixture was allowed to warm to rt overnight. NH₄Cl (sat'd) was added and extracted with EA. The organic layers were dried over sodium sulfate, filtered and concentrated. The residue was purified by silica gel column chromatography (EA/Hept. (0-100%) to give tert-butyl 3-(trifluoromethylsulfonyloxy)-8-azabicyclo[3.2.1]oct-2-ene-8-carboxylate (473 mg, 1.32 mmol, 99% yield) as a colorless oil.

### 2. Preparation of tert-butyl 3-(4,4,5,5-tetramethyl-1,3,2-dioxaborolan-2-yl)-8-azabicyclo[3.2.1]oct-2-ene-8-carboxylate

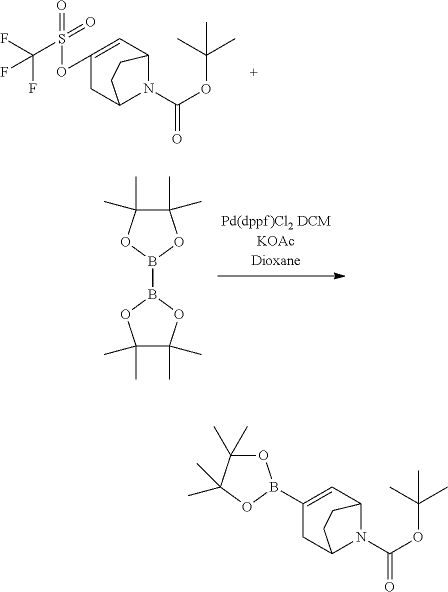

To a solution of tert-butyl 3-(trifluoromethylsulfonyloxy)-8-azabicyclo[3.2.1]oct-2-ene-8-carboxylate (573 mg, 1.60 mmol) and 4,4,5,5-tetramethyl-2-(4,4,5,5-tetramethyl-1,3,2-dioxaborolan-2-yl)-1,3,2-dioxaborolane (814.38 mg, 3.21 mmol) in dioxane (5 mL) was added Pd(dppf)Cl₂ DCM (130.95 mg, 160.35 umol) and KOAc (472.10 mg, 4.81 mmol) The mixture was stirred at 90° C. under N₂ for 12 hours. After filtration through celite, the reaction mixture was concentrated under vacuum to give a crude, which was purified by silica gel column chromatography (PE/EA=7/3) to give tert-butyl 3-(4,4,5,5-tetramethyl-1,3,2-dioxaborolan-2-yl)-8-azabicyclo[3.2.1]oct-2-ene-8-carboxylate (372 mg, 1.11 mmol, 69% yield) as colorless oil.

### 3. Preparation of tert-butyl 3-[6-(1-methylpyrazol-4-yl)pyrazolo[1,5-a]pyrazin-4-yl]-8-azabicyclo[3.2.1]oct-2-ene-8-carboxylate

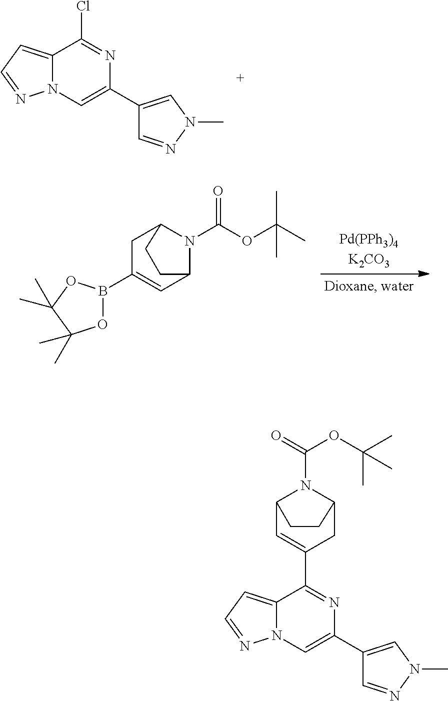 [SMILES: Cn1cc(-c2cn3nccc3c(Cl)n2)cn1 ; CC(C)(C)OC(=O)N1C2C=C(B3OC(C)(C)C(C)(C)O3)CC1CC2 ; Cn1cc(-c2cn3nccc3c(C3=CC4CCC(C3)N4C(=O)OC(C)(C)C)n2)cn1]

4-chloro-6-(1-methylpyrazol-4-yl)pyrazolo[1,5-a]pyrazine (200 mg, 855.96 umol), tert-butyl 3-(4,4,5,5-tetramethyl-1,3,2-dioxaborolan-2-yl)-8-azabicyclo[3.2.1]oct-2-ene-8-carboxylate (430.43 mg, 1.28 mmol), K₂CO₃ (354.91 mg, 2.57 mmol), tetrakis(triphenyl phosphine)palladium(0) (197.82 mg, 171.19 umol) in water (0.5 mL) and dioxane (3 mL) was degassed and heated to 95° C. for 16 h. After cooling to rt, the mix was diluted with EtOAc and filtrated through celite. The concentrated residue was purified by silica gel column chromatography (EtOAc/DCM 0-100%) to give tert-butyl 3-[6-(1-methylpyrazol-4-yl)pyrazolo[1,5-a]pyrazin-4-yl]-8-azabicyclo[3.2.1]oct-2-ene-8-carboxylate (372 mg, 915.17 umol, 106% yield) as a white solid. LCMS (ESI+): m/z calcd. for C₂₂H₂₇N₆O₂ [M+H]⁺, 407.2; found, 407.3. Rt=0.99 min.

### 4. Preparation of 4-(8-azabicyclo[3.2.1]oct-2-en-3-yl)-6-(1-methylpyrazol-4-yl)pyrazolo[1,5-a]pyrazine

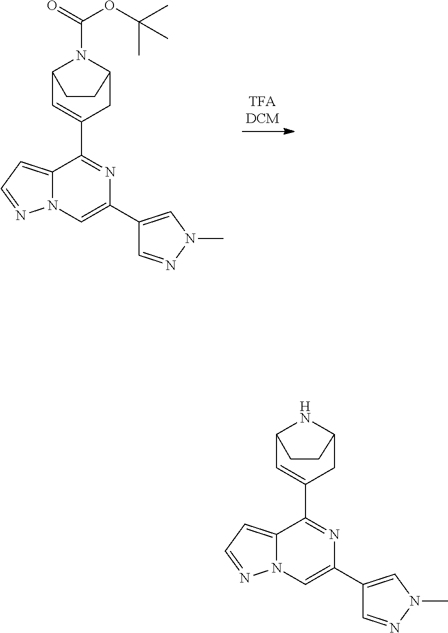 [SMILES: Cn1cc(-c2cn3nccc3c(C3=CC4CCC(C3)N4C(=O)OC(C)(C)C)n2)cn1 ; Cn1cc(-c2cn3nccc3c(C3=CC4CCC(C3)N4)n2)cn1]

tert-butyl 3-[6-(1-methylpyrazol-4-yl)pyrazolo[1,5-a]pyrazin-4-yl]-8-azabicyclo[3.2.1]oct-2-ene-8-carboxylate (376 mg, 925.01 umol) in DCM was added TFA (105.47 mg, 925.01 umol, 70.83 uL) and stirred at rt for 1 h. The crude was concentrated and used as is for the next step. LCMS (ESI+): m/z calcd. for C₁₇H₁₉N₆[M+H]⁺, 307.2; found, 307.1. Rt=0.64 min.

### 5. Preparation of 4-(8-azabicyclo[3.2.1]octan-3-yl)-6-(1-methylpyrazol-4-yl)pyrazolo[1,5-a]pyrazine

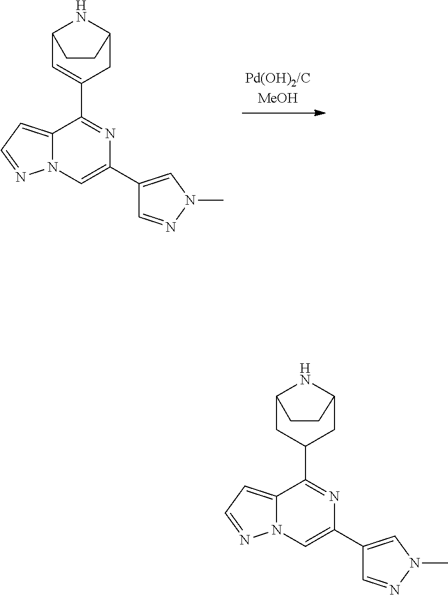 [SMILES: Cn1cc(-c2cn3nccc3c(C3=CC4CCC(C3)N4)n2)cn1 ; Cn1cc(-c2cn3nccc3c(C3CC4CCC(C3)N4)n2)cn1]

4-(8-azabicyclo[3.2.1]oct-2-en-3-yl)-6-(1-methylpyrazol-4-yl)pyrazolo[1,5-a]pyrazine (481 mg, 1.57 mmol) in MeOH (6 mL) was added Pd(OH)₂/C (220.49 mg, 314.01 umol, 20% purity) and stirred under H₂ at 60 psi at rt for 16 h. After filtration through celite, the concentrated residue was used as is for the next step. LCMS (ESI+): m/z calcd. for C₁₇H₂₁N₆ [M+H]⁺, 309.2; found, 309.1. Rt=0.47 min.

### 6. Preparation of 1-[3-[6-(1-methylpyrazol-4-yl)pyrazolo[1,5-a]pyrazin-4-yl]-8-azabicyclo[3.2.1]octan-8-yl]prop-2-en-1-one

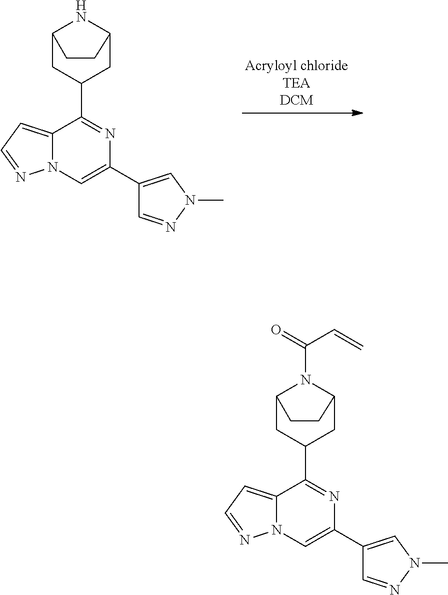 [SMILES: Cn1cc(-c2cn3nccc3c(C3CC4CCC(C3)N4)n2)cn1 ; C=CC(=O)N1C2CCC1CC(c1nc(-c3cnn(C)c3)cn3nccc13)C2]

4-(8-azabicyclo[3.2.1]octan-3-yl)-6-(1-methylpyrazol-4-yl)pyrazolo[1,5-a]pyrazine (446 mg, 1.06 mmol, TFA) in DCM (5 mL) was added TEA (214.20 mg, 2.12 mmol, 295.04 uL) and stirred for 5 min. After cooling to 0° C., acryloyl chloride (114.95 mg, 1.27 mmol, 103.19 uL) was added and stirred for 3 min. The reaction was quenched with sat. aq. NaHCO₃ and extracted with DCM. The organic layer was dried over Na2SO₄ and the concentrated residue was purified by silica gel column chromatography (EtOAc/MeOH 0-15%) then prep HPLC to give 1-[3-[6-(1-methylpyrazol-4-yl)pyrazolo[1,5-a]pyrazin-4-yl]-8-azabicyclo[3.2.1]octan-8-yl]prop-2-en-1-one (45.3 mg, 118.74 umol, 11% yield, 95% purity) as a white solid. LCMS (ESI+): m/z calcd. for C₂₀H₂₃N₆O [M+H]⁺, 363.2; found, 363.2. Rt=0.60 min. ¹H NMR (400 MHz, CDCl₃, d): 8.44-8.47 (m, 1H), 7.99-8.06 (m, 1H), 7.92-7.98 (m, 1H), 7.86-7.90 (m, 1H), 6.64-6.77 (m, 1H), 6.55-6.63 (m, 1H), 6.43-6.51 (m, 1H), 5.72-5.80 (m, 1H), 4.82-5.00 (m, 1H), 4.42-4.58 (m, 1H), 3.98-4.04 (m, 3H), 3.92 (br s, 1H), 1.86-3.00 (m, 8H).

### Example 12. 1-[2-methyl-4-[6-(1-methylpyrazol-4-yl)pyrazolo[1,5-a]pyrazin-4-yl]-1-piperidyl]prop-2-en-1-one

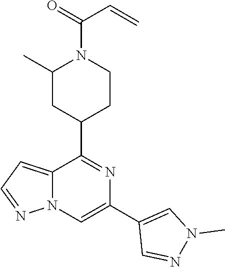 [SMILES: C=CC(=O)N1CCC(c2nc(-c3cnn(C)c3)cn3nccc23)CC1C]

1-[2-methyl-4-[6-(1-methylpyrazol-4-yl)pyrazolo[1,5-a]pyrazin-4-yl]-1-piperidyl]prop-2-en-1-one was prepared in a similar way as Example 1 starting from tert-butyl 2-methyl-4-oxo-piperidine-1-carboxylate instead. Weight: 22.6 mg, 95% purity as a white solid. LCMS (ESI+): m/z calcd. for C₁₉H₂₃N₆O [M+H]⁺, 351.2; found, 351.1. Rt=0.61 min. ¹H NMR (400 MHz, CDCl₃, d): 8.43-8.47 (m, 1H), 7.96-8.01 (m, 1H), 7.87-7.93 (m, 2H), 6.71-6.79 (m, 1H), 6.60-6.69 (m, 1H), 6.37-6.44 (m, 1H), 5.70-5.77 (m, 1H), 4.20-4.56 (m, 2H), 3.98-4.03 (m, 3H), 3.49 (br s, 1H), 3.33-3.43 (m, 1H), 2.19-2.34 (m, 2H), 2.02-2.14 (m, 2H), 1.16-1.28 (m, 3H).

### Example 13. 1-[3-[6-(1-methylpyrazol-4-yl)pyrazolo[1,5-a]pyrazin-4-yl]-9-azabicyclo[3.3.1]nonan-9-yl]prop-2-en-1-one

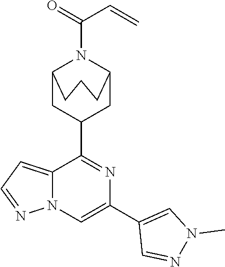 [SMILES: C=CC(=O)N1C2CCCC1CC(c1nc(-c3cnn(C)c3)cn3nccc13)C2]

1-[3-[6-(1-methylpyrazol-4-yl)pyrazolo[1,5-a]pyrazin-4-yl]-9-azabicyclo[3.3.1]nonan-9-yl]prop-2-en-1-one was prepared in a similar way as Example 1 starting from tert-butyl 3-oxo-9-azabicyclo[3.3.1]nonane-9-carboxylate instead. Weight: 96 mg, 95% purity as a white solid. LCMS (ESI+): m/z calcd. for C₂₁H₂₅N₆O [M+H]⁺, 377.2; found, 377.3. Rt=0.66 min. ¹H NMR (400 MHz, CDCl₃, d): 8.43 (s, 1H), 7.84-8.01 (m, 3H), 6.72-6.80 (m, 1H), 6.59-6.72 (m, 1H), 6.30-6.42 (m, 1H), 5.67-5.79 (m, 1H), 5.04-5.24 (m, 1H), 4.35-4.54 (m, 1H), 3.96-4.02 (m, 3H), 2.98-3.13 (m, 1H), 2.32-2.53 (m, 2H), 1.85-2.21 (m, 4H), 1.55-1.84 (m, 4H).

### Example 14. 1-[5-[6-(1-methylpyrazol-4-yl)pyrazolo[1,5-a]pyrazin-4-yl]-2-azabicyclo[2.2.2]octan-2-yl]prop-2-en-1-one

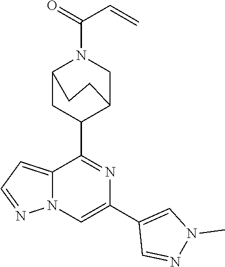 [SMILES: C=CC(=O)N1CC2CCC1CC2c1nc(-c2cnn(C)c2)cn2nccc12]

1-[5-[6-(1-methylpyrazol-4-yl)pyrazolo[1,5-a]pyrazin-4-yl]-2-azabicyclo[2.2.2]octan-2-yl]prop-2-en-1-one was prepared in a similar way as Example 1 starting from tert-butyl 5-oxo-2-azabicyclo[2.2.2]octane-2-carboxylate instead. Weight: 4.9 mg, 95% purity as a white solid. LCMS (ESI+): m/z calcd. for C₂₀H₂₃N₆O [M+H]⁺, 363.2; found, 363.2. Rt=0.62 min. ¹H NMR (400 MHz, CDCl₃, δ): 8.44-8.51 (m, 1H), 7.85-8.04 (m, 3H), 6.68-6.76 (m, 1 H), 6.54-6.67 (m, 1H), 6.34-6.49 (m, 1H), 5.57-5.75 (m, 1H), 4.17-4.24 (m, 1H), 3.99-4.04 (m, 3H), 3.53-3.85 (m, 2H), 2.14-3.00 (m, 3H), 1.44-1.97 (m, 5H).

### Example 15. 1-[3-methyl-4-[6-(1-methylpyrazol-4-yl)pyrazolo[1,5-a]pyrazin-4-yl]-1-piperidyl]prop-2-en-1-one

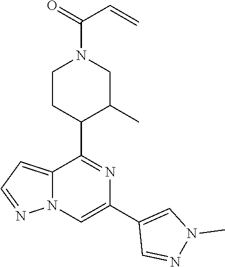 [SMILES: C=CC(=O)N1CCC(c2nc(-c3cnn(C)c3)cn3nccc23)C(C)C1]

1-[3-methyl-4-[6-(1-methylpyrazol-4-yl)pyrazolo[1,5-a]pyrazin-4-yl]-1-piperidyl]prop-2-en-1-one was prepared in a similar way as Example 1 starting from tert-butyl 3-methyl-4-oxo-piperidine-1-carboxylate instead. Weight: 51 mg, 95% purity as a white solid. LCMS (ESI+): m/z calcd. for C₁₉H₂₃N₆O [M+H]⁺, 351.2; found, 351.1. Rt=0.61 min. ¹H NMR (400 MHz, CDCl₃, δ): 8.45-8.48 (m, 1H), 7.97-8.00 (m, 1H), 7.86-7.96 (m, 2H), 6.75-6.79 (m, 1H), 6.58-6.71 (m, 1H), 6.31-6.39 (m, 1H), 5.69-5.77 (m, 1H), 3.99-4.02 (m, 3H), 3.03-3.71 (m, 4H), 2.25-2.68 (m, 3H), 1.87-1.96 (m, 1H), 0.75-0.85 (m, 3H).

### Examples 16-19

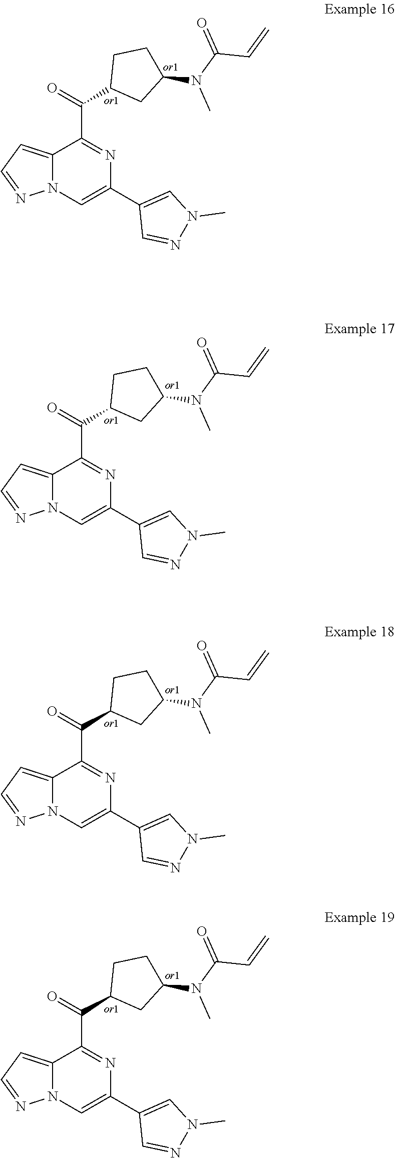

### 1. Preparation of Rac-3-(methylamino)cyclopentan-1-ol

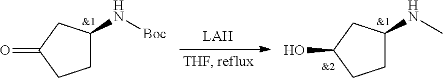

Rac-tert-butyl (3-oxocyclopentyl)carbamate (3 g, 15.06 mmol, 1 eq.) in THF (100 mL) was added LAH (1.14 g, 30.11 mmol, 2 eq.) at 0° C. under N₂. The mixture was then heated to 70° C. and stirred at 70° C. for 2.5 h. TLC (PE/EtOAc=1/1) showed the starting material was consumed completely and a new more polar spot was detected. Water (5 mL) was added dropwise and Na2SO₄ (3 g) was added the mixture. The mixture was filtered and the filtrate was concentrated to give rac-3-(methylamino)cyclopentan-1-ol (1.7 g, crude) as a colorless oil. ¹H NMR: (400 MHz, CDCl₃) δ=4.37-4.14 (m, 1H), 3.24-3.08 (m, 1H), 2.32 (d, J=5.6 Hz, 3H), 2.06-1.91 (m, 1H), 1.88-1.72 (m, 2H), 1.65-1.46 (m, 2H), 1.39-1.26 (m, 1H).

### 2. Preparation of rac-tert-butyl (3-hydroxycyclopentyl)(methyl)carbamate

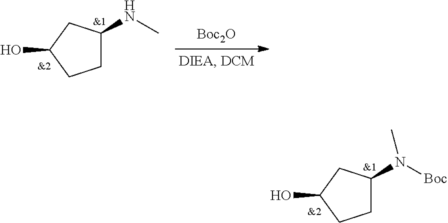

Rac-3-(methylamino)cyclopentan-1-ol (1.7 g, 14.76 mmol, 1 eq.), DIEA (3.82 g, 29.52 mmol, 5.14 mL, 2 eq.) and Boc₂O (6.44 g, 29.52 mmol, 2 eq.) in DCM (50 mL) was stirred at 20° C. for 12 h. TLC (PE/EtOAc=1/1) showed the starting material was consumed completely and a new less polar spot was found. LCMS showed the desired MS was detected. Water (30 mL) was added and the mixture was extracted with DCM (60 mL×3). The organics were dried over Na2SO₄, filtered and concentrated to get a crude, which was purified by silica gel column chromatography eluting with EtOAc in PE from 0% to 50% to 80% to give rac-tert-butyl (3-hydroxycyclopentyl)(methyl)carbamate (2.8 g, 13.01 mmol, 88.11% yield) as a colorless oil. LCMS: (M+H⁺-56: 160.1). ¹H NMR: (400 MHz, CDCl₃) δ=4.78-4.31 (m, 1H), 4.26-4.13 (m, 1H), 2.84-2.68 (m, 3H), 1.90-1.73 (m, 4H), 1.64-1.54 (m, 2H), 1.47-1.41 (m, 9H).

### 3. Preparation of rac-tert-butyl methyl(3-oxocyclopentyl)carbamate

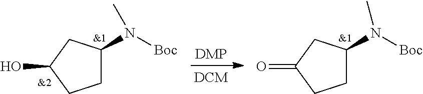

To the mixture of rac-tert-butyl (3-hydroxycyclopentyl)(methyl)carbamate (2.8 g, 13.01 mmol, 1 eq.) in DCM (10 mL) was added DMP (8.27 g, 19.51 mmol, 1.5 eq.) at 20° C. under N₂. The mixture was stirred at 20° C. for 16 h. Much white solid was separated out. TLC (PE/EtOAc=3/1) showed the starting material was consumed completely and a new less spot was found. The mixture was filtered and the filtrate was washed with water (30 mL×2), brine (30 mL), dried over Na2SO₄, filtered and the filtrate was concentrated to get a crude, which purified by silica gel column chromatography eluting with EtOAc in PE from 0% to 30% to give rac-tert-butyl methyl(3-oxocyclopentyl)carbamate (2.3 g, 10.78 mmol, 82.92% yield) as a colorless oil. ¹H NMR: (400 MHz, CDCl₃) δ=4.69 (s, 1H), 2.77 (s, 3H), 2.47-2.33 (m, 2H), 2.28-2.14 (m, 3H), 1.99-1.91 (m, 1H), 1.45 (s, 9H).

### 4. Preparation of rac-tert-butyl (Z)-methyl(3-(2-tosylhydrazineylidene)cyclopentyl) carbamate

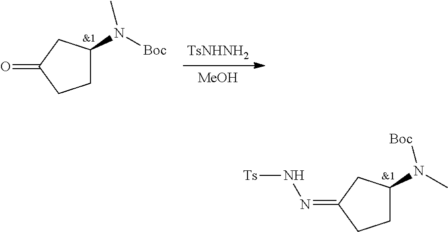 [SMILES: "TsNHNH2" ; "MeOH" ; CN(C(=O)OC(C)(C)C)[C@H]1CCC(=O)C1 ; Cc1ccc(S(=O)(=O)N/N=C2/CC[C@H](N(C)C(=O)OC(C)(C)C)C2)cc1]

To a solution of rac-tert-butyl methyl(3-oxocyclopentyl)carbamate (1 g, 4.69 mmol, 1 eq.) in MeOH (10 mL) was added TsNHNH₂ (873.21 mg, 4.69 mmol, 1 eq.) at 20° C. The mixture was stirred at 20° C. for 40 min. LCMS showed the starting material was consumed completely and a major peak with desired MS was detected. The solvent was removed below 25° C. to get crude rac-tert-butyl (Z)-methyl(3-(2-tosylhydrazineylidene)cyclopentyl) carbamate (1.7 g, crude) as a colorless oil. LCMS: (M+H+: 326.1).

### 5. Preparation of rac-tert-butyl methyl(3-(6-(1-methyl-1H-pyrazol-4-yl)pyrazolo[1,5-a]pyrazine-4-carbonyl)cyclopentyl)carbamate

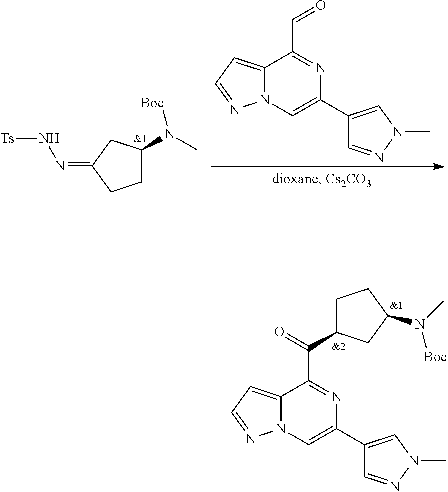 [SMILES: Cc1ccc(S(=O)(=O)N/N=C2/CC[C@H](N(C)C(=O)OC(C)(C)C)C2)cc1 ; Cn1cc(-c2cn3nccc3c(C=O)n2)cn1 ; CN(C(=O)OC(C)(C)C)[C@@H]1CC[C@H](C(=O)c2nc(-c3cnn(C)c3)cn3nccc23)C1]

To a solution of rac-tert-butyl (Z)-methyl(3-(2-tosylhydrazineylidene)cyclopentyl) carbamate (1.7 g, 4.46 mmol, 1 eq.) in dioxane (30 mL) was added Cs₂CO₃ (4.36 g, 13.37 mmol, 3 eq.) at 20° C. Then compound 6 (1.62 g, 7.13 mmol, 1.6 eq.) was added to the mixture at 20° C. slowly. Then the mixture was stirred at 110° C. for 16 h under N₂. LCMS showed several peaks were detected and a peak with desired MS was found. The solvent was removed to give a crude, which was purified by silica gel column chromatography eluting with EtOAc in PE from 0% to 50% to 100% to give rac-tert-butyl methyl(3-(6-(1-methyl-1H-pyrazol-4-yl)pyrazolo[1,5-a]pyrazine-4-carbonyl)cyclopentyl)carbamate (302 mg, 0.604 mmol, 13.57% yield, 85% purity) as a colorless oil. LCMS: (M+H⁺: 425.2). ¹H NMR: (500 MHz, CDCl₃) δ=8.73-8.69 (m, 1H), 8.14-8.11 (m, 1H), 8.00-7.92 (m, 2H), 7.46-7.44 (m, 1H), 4.63-4.35 (m, 2H), 4.17-4.09 (m, 1H), 4.03-4.00 (m, 3H), 3.86-3.73 (m, 1H), 2.86-2.78 (m, 3H), 2.23-2.14 (m, 1H), 1.99-1.84 (m, 3H), 1.49-1.47 (m, 9H).

### 6. Preparation of rac-(6-(1-methyl-H-pyrazol-4-yl)pyrazolo[1,5-a]pyrazin-4-yl)(3-(methylamino)cyclopentyl)methanone hydrochloride

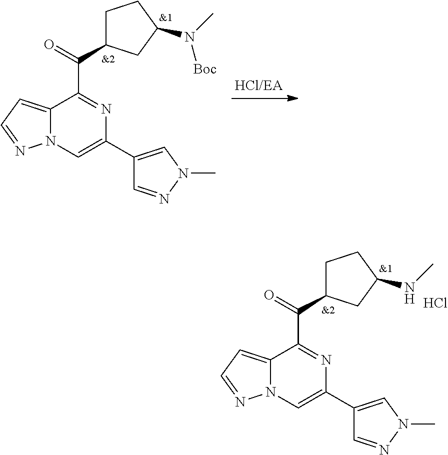 [SMILES: CN(C(=O)OC(C)(C)C)[C@@H]1CC[C@H](C(=O)c2nc(-c3cnn(C)c3)cn3nccc23)C1 ; CN[C@@H]1CC[C@H](C(=O)c2nc(-c3cnn(C)c3)cn3nccc23)C1 ; Cl]

The mixture of rac-tert-butyl methyl(3-(6-(1-methyl-1H-pyrazol-4-yl)pyrazolo[1,5-a]pyrazine-4-carbonyl)cyclopentyl)carbamate (302 mg, 0.711 mmol, 1 eq.) in DCM (1 mL) was added HCl in EtOAc (4 M, 6 mL). The mixture was stirred at 25° C. for 1 hour. LCMS showed the starting material was consumed completely and a peak with desired MS was detected. The reaction mixture was concentrated to give rac-(6-(1-methyl-1H-pyrazol-4-yl)pyrazolo[1,5-a]pyrazin-4-yl)(3-(methylamino)cyclopentyl)methanone hydrochloride (230 mg, 0.709 mmol, 99.66% yield) as a yellow solid, which was used to next step directly. LCMS: (M+H⁺: 325.1).

### 7. Preparation of rac-N-methyl-N-(3-(6-(1-methyl-1H-pyrazol-4-yl)pyrazolo[1,5-a]pyrazine-4-carbonyl)cyclopentyl)acrylamide

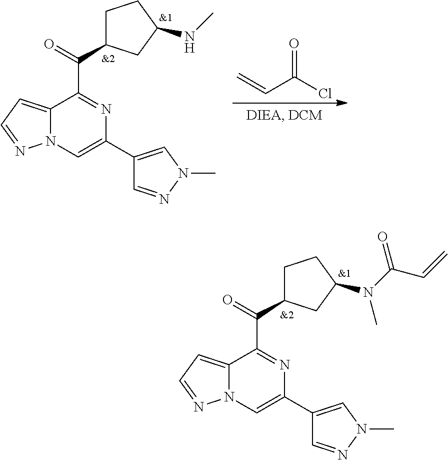

To the mixture of rac-(6-(1-methyl-H-pyrazol-4-yl)pyrazolo[1,5-a]pyrazin-4-yl)(3-(methylamino)cyclopentyl)methanone hydrochloride (230 mg, 0.709 mmol, 1 eq.) and DIEA (274.92 mg, 2.13 mmol, 3 eq.) in DCM (15 mL) was added compound 9 (64.17 mg, 0.709 mmol, 1 eq.) at 0° C. The mixture was stirred at 0° C. for 2 min. LCMS showed the starting material was consumed completely and a peak with desired MS was detected. MeOH (1 mL) was added dropwise. The resulting mixture was stirred at 25° C. for 10 min. The solvent was removed to get a crude, which was purified by prep HPLC (Column: Welch Xtimate C18 150×25 mm×5 um; Condition: water (NH₄HCO₃)-ACN, Begin B 35, End B 55, Gradient Time (min) 12, 100% B Hold Time (min) 2, Flow Rate (mL/min) 25) to give rac-N-methyl-N-(3-(6-(1-methyl-1H-pyrazol-4-yl)pyrazolo[1,5-a]pyrazine-4-carbonyl)cyclopentyl)acrylamide (200 mg, 0.528 mmol, 74.54% yield) as a white solid. LCMS: (M+H⁺: 379.1).

### 8. General procedure for separation of N-methyl-N-(3-(6-(1-methyl-1H-pyrazol-4-yl)pyrazolo[1,5-a]pyrazine-4-carbonyl)cyclopentyl)acrylamide

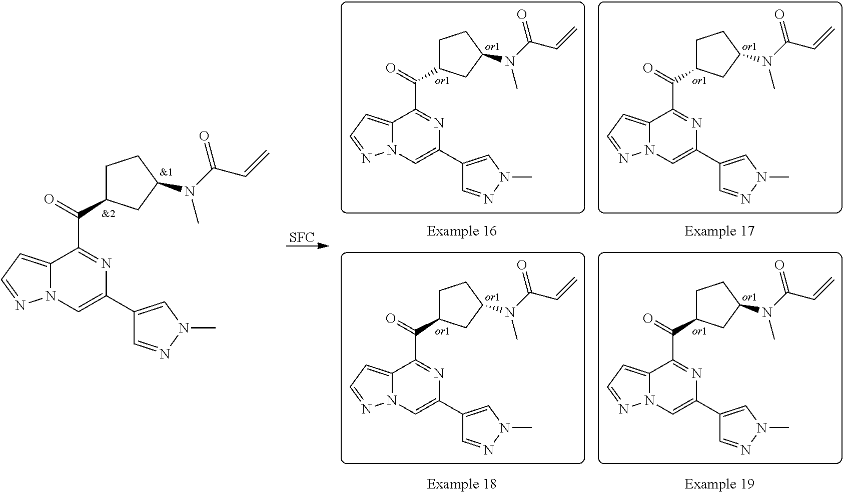

N-methyl-N-(3-(6-(1-methyl-1H-pyrazol-4-yl)pyrazolo[1,5-a]pyrazine-4-carbonyl) cyclopentyl)acrylamide (120 mg, 0.305 mmol, 1 eq.) as a mixture of diastereomers was separated by SFC (Column: DAICEL CHIRALPAK IG (250 mm×30 mm, 10 um); Condition: 0.1% NH₃H₂O MeOH, Begin B 55%, End B 55%, Gradient Time (min), 100% B Hold Time (min), Flow Rate (mL/min) 80) to give Example 16 (15.2 mg, Rt=2.256 min) as a yellow solid and the mixture of Example 17 and Example 18 (40 mg) as a yellow solid and the impure Example 19 (17 mg). The mixture of Example 17 and Example 18 (40 mg) was separated further by SFC (Column: DAICEL CHIRALPAK IG (250 mm×30 mm, 10 um); Condition: 0.1% NH₃H₂O EtOH, Begin B 60%, End B 60%, Gradient Time (min), 100% B Hold Time (min), Flow Rate (mL/min) 80) to give Example 17 (11.6 mg, Rt=2.437 min) as a yellow solid and Example 18 (12.6 mg, Rt=2.409 min) as a yellow solid. The impure Example 19 (17 mg) was purified further by prep HPLC (Column: Waters Xbridge BEH C18 100×25 mm×5 um; Condition: water (FA)-ACN, Begin B 20, End B 40, Gradient Time (min) 12, 100% B Hold Time (min) 2, Flow Rate (mL/min) 25) to give Example 19 (11.6 mg, Rt=2.630 min) as a yellow solid.

### Spectra of Example 16

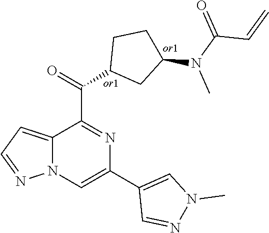

LCMS: (M+H⁺: 379.3). HPLC: (Purity: 100%). SFC: (ee: 96.64%). ¹H NMR: (500 MHz, METHANOL-d₄) δ=8.96 (s, 1H), 8.14 (s, 1H), 8.06 (s, 1H), 7.99 (s, 1H), 7.32 (d, J=1.5 Hz, 1H), 6.80-6.59 (m, 1H), 6.09 (t, J=17.5 Hz, 1H), 5.66-5.63 (m, 1H), 4.99-4.88 (m, 0.5H), 4.57-4.46 (m, 1.5H), 3.93-3.86 (m, 3H), 3.04-2.85 (m, 3H), 2.29-2.09 (m, 2H), 2.06-1.74 (m, 4H).

### Spectra of Example 17

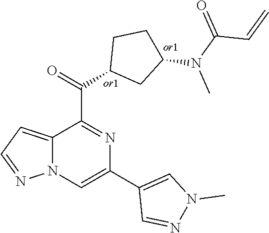 [SMILES: C=CC(=O)N(C)[C@H]1CC[C@@H](C(=O)c2nc(-c3cnn(C)c3)cn3nccc23)C1]

LCMS: (M+H⁺: 379.3). HPLC: (Purity: 100%). SFC: (ee: 99.26%). ¹H NMR: (500 MHz, METHANOL-d₄) δ=8.94 (s, 1H), 8.13 (s, 1H), 8.05 (d, J=2.0 Hz, 1H), 7.97 (s, 1H), 7.31 (s, 1H), 6.86-6.59 (m, 1H), 6.11 (t, J=18.5 Hz, 1H), 5.66-5.63 (m, 1H), 5.12-4.99 (m, 0.5H), 4.66-4.54 (m, 0.5H), 4.42 (d, J=5.5 Hz, 1H), 3.94-3.83 (m, 3H), 3.04-2.82 (m, 3H), 2.18-2.00 (m, 2H), 1.99-1.63 (m, 4H).

### Spectra of Example 18

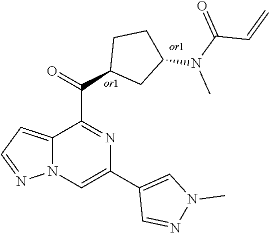 [SMILES: C=CC(=O)N(C)[C@H]1CC[C@H](C(=O)c2nc(-c3cnn(C)c3)cn3nccc23)C1]

LCMS: (M+H⁺: 379.3). HPLC: (Purity: 100%). SFC: (Purity: 100%). ¹H NMR: (500 MHz, METHANOL-d₄) δ=8.96 (s, 1H), 8.14 (s, 1H), 8.06 (s, 1H), 7.99 (s, 1H), 7.32 (d, J=1.5 Hz, 1H), 6.80-6.59 (m, 1H), 6.09 (t, J=17.5 Hz, 1H), 5.66-5.63 (m, 1H), 4.99-4.88 (m, 0.5H), 4.57-4.46 (m, 1.5H), 3.93-3.86 (m, 3H), 3.04-2.85 (m, 3H), 2.29-2.09 (m, 2H), 2.06-1.74 (m, 4H).

### Spectra of Example 19

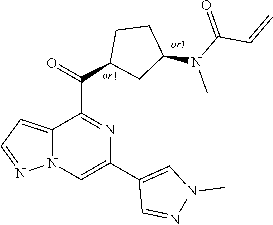 [SMILES: C=CC(=O)N(C)[C@@H]1CC[C@H](C(=O)c2nc(-c3cnn(C)c3)cn3nccc23)C1]

LCMS: (M+H⁺: 379.3). HPLC: (Purity: 100%). SFC: (Purity: 97.26%). ¹H NMR: (500 MHz, METHANOL-d₄) δ=8.94 (s, 1H), 8.13 (s, 1H), 8.05 (d, J=2.0 Hz, 1H), 7.97 (s, 1H), 7.31 (s, 1H), 6.86-6.59 (m, 1H), 6.11 (t, J=18.5 Hz, 1H), 5.66-5.63 (m, 1H), 5.12-4.99 (m, 0.5H), 4.66-4.54 (m, 0.5H), 4.42 (d, J=5.5 Hz, 1H), 3.94-3.83 (m, 3H), 3.04-2.82 (m, 3H), 2.18-2.00 (m, 2H), 1.99-1.63 (m, 4H).

### Examples 20-23

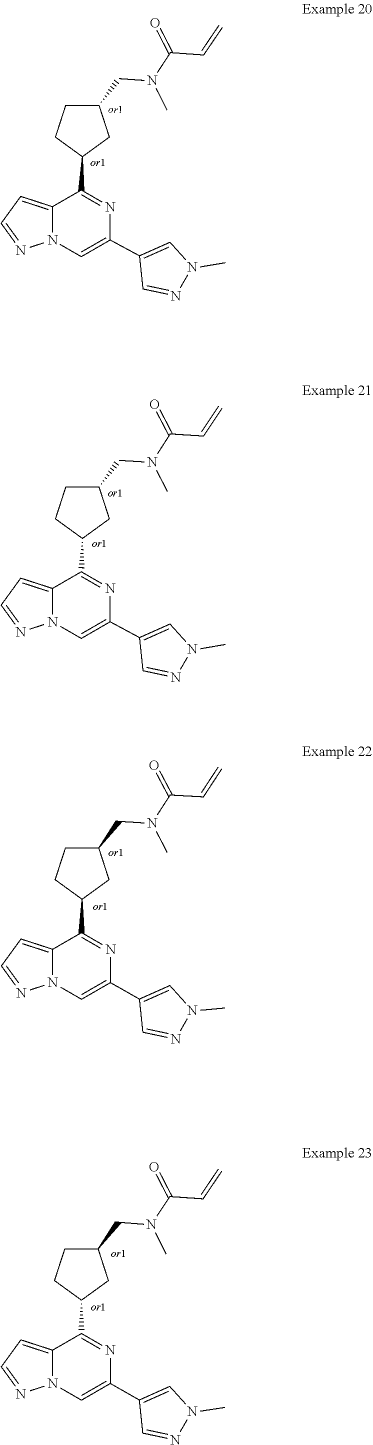 [SMILES: C=CC(=O)N(C)C[C@H]1CC[C@H](c2nc(-c3cnn(C)c3)cn3nccc23)C1 ; C=CC(=O)N(C)C[C@H]1CC[C@@H](c2nc(-c3cnn(C)c3)cn3nccc23)C1 ; C=CC(=O)N(C)C[C@@H]1CC[C@H](c2nc(-c3cnn(C)c3)cn3nccc23)C1 ; C=CC(=O)N(C)C[C@@H]1CC[C@@H](c2nc(-c3cnn(C)c3)cn3nccc23)C1]

### 1. Preparation of rac-methyl 3-(6-(1-methyl-1H-pyrazol-4-yl)pyrazolo[1,5-a]pyrazin-4-yl)cyclopent-3-ene-1-carboxylate

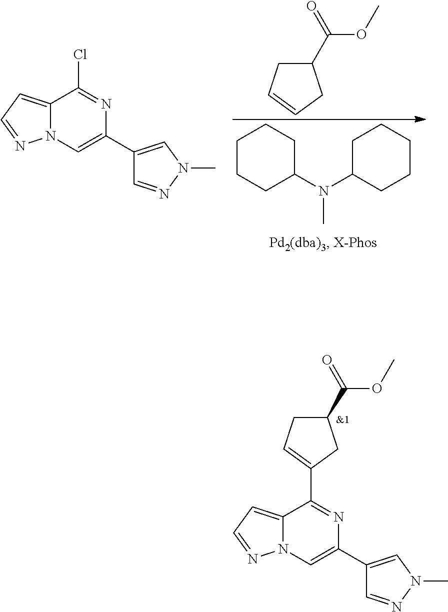

The mixture of 4-chloro-6-(1-methylpyrazol-4-yl)pyrazolo[1,5-a]pyrazine (1 g, 4.28 mmol, 1.00 eq.), methyl cyclopent-3-ene-1-carboxylate (971.83 mg, 7.70 mmol, 1.80 eq.), Pd(dba)₂ (246.09 mg, 427.98 mol, 0.10 eq.), XPhos (306.04 mg, 641.97 mol, 0.15 eq.) and N,N-dicyclohexylmethylamine (1.67 g, 8.56 mmol, 2.00 eq.) in dioxane (24 mL) was bubbled with N₂ for 1 min. Then the mixture was stirred at 100° C. for 16 hours. LCMS showed the starting material was consumed completely and a major peak with desired MS was detected. The mixture was cooled to 15° C. and the solvent was removed to get a residue, which was purified by silica gel column chromatography eluting with EtOAc in PE from 0% to 50% to 100% to give rac-methyl 3-(6-(1-methyl-1H-pyrazol-4-yl)pyrazolo[1,5-a]pyrazin-4-yl)cyclopent-3-ene-1-carboxylate (1.3 g, 93.94% yield) as orange oil. LCMS: (M+H⁺: 324.3). ¹H NMR: (400 MHz, METHANOL-d₄) δ=8.61 (s, 1H), 8.07 (s, 1H), 7.98-7.94 (m, 2H), 6.92-6.86 (m, 1H), 6.09-5.94 (m, 1H), 4.10-3.91 (m, 6H), 3.74-3.70 (m, 3H), 3.11-3.06 (m, 1H), 2.68-2.66 (m, 1H).

### 2. Preparation of rac-methyl 3-(6-(1-methyl-1H-pyrazol-4-yl)pyrazolo[1,5-a]pyrazin-4-yl)cyclopentane-1-carboxylate

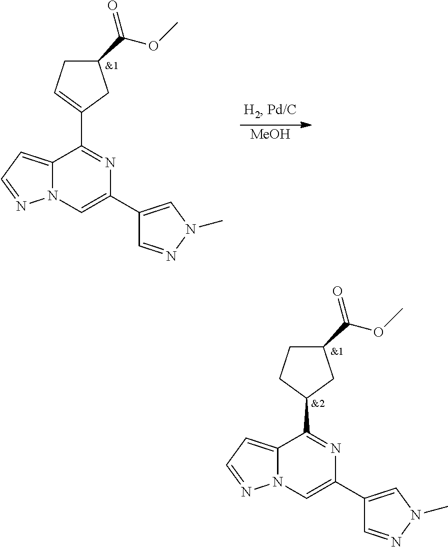 [SMILES: "MeOH" ; COC(=O)[C@@H]1CC=C(c2nc(-c3cnn(C)c3)cn3nccc23)C1 ; COC(=O)[C@@H]1CC[C@H](c2nc(-c3cnn(C)c3)cn3nccc23)C1]

To a solution of rac-methyl 3-(6-(1-methyl-1H-pyrazol-4-yl)pyrazolo[1,5-a]pyrazin-4-yl)cyclopent-3-ene-1-carboxylate (700.00 mg, 2.16 mmol, 1.00 eq.) in MeOH (60 mL) was added Pd/C (230.38 mg, 216.48 mol, 10% purity, 0.10 eq.) at 20° C. The mixture was stirred with H₂ at 20° C. and 15 psi for 8 hours. LCMS showed the starting material was consumed completely and a major peak with desired MS was detected. The reaction mixture was filtrated and concentrated under vacuum to give the crude rac-methyl 3-(6-(1-methyl-1H-pyrazol-4-yl)pyrazolo[1,5-a]pyrazin-4-yl)cyclopentane-1-carboxylate (540 mg, crude), which was used to next step directly. LCMS (M+H⁺: 326.1).

### 3. Preparation of rac-3-(6-(1-methyl-H-pyrazol-4-yl)pyrazolo[1,5-a]pyrazin-4-yl)cyclopentane-1-carboxylic acid

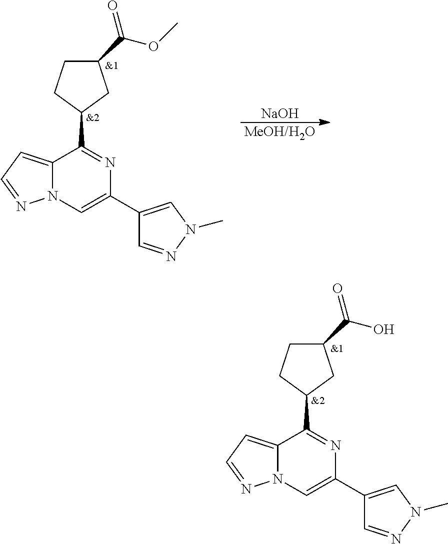 [SMILES: COC(=O)[C@@H]1CC[C@H](c2nc(-c3cnn(C)c3)cn3nccc23)C1 ; "NaOH" ; "MeOH/H2O" ; Cn1cc(-c2cn3nccc3c([C@H]3CC[C@@H](C(=O)O)C3)n2)cn1]

To a solution of rac-methyl 3-(6-(1-methyl-1H-pyrazol-4-yl)pyrazolo[1,5-a]pyrazin-4-yl)cyclopentane-1-carboxylate (1.00 g, 3.07 mmol, 1.00 eq.) in MeOH (30 mL) and water (10 mL) was added NaOH (147.52 mg, 3.69 mmol, 1.20 eq.) at 25° C. The reaction mixture was stirred at 25° C. for 8 hours. LCMS showed that the starting material was consumed completely. To the reaction mixture was added HCl (2 M, 2 mL) to adjust pH=5-6 at 25° C. The crude material was concentrated under vacuum to give the crude rac-3-(6-(1-methyl-1H-pyrazol-4-yl)pyrazolo[1,5-a]pyrazin-4-yl)cyclopentane-1-carboxylic acid (950 mg, crude) as a yellow solid. LCMS: (M+H⁺: 312.1).

### 4. Preparation of rac-N-methyl-3-(6-(1-methyl-1H-pyrazol-4-yl)pyrazolo[1,5-a]pyrazin-4-yl)cyclopentane-1-carboxamide

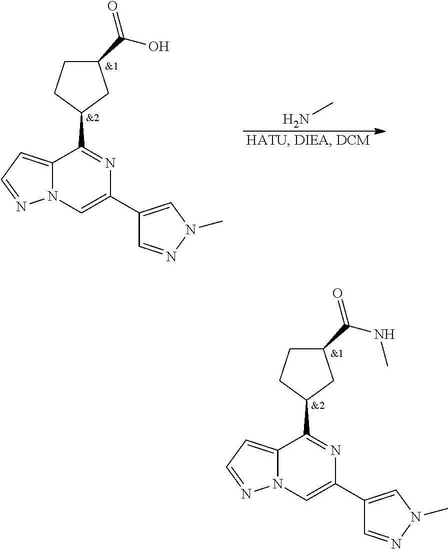

To a solution of rac-3-(6-(1-methyl-1H-pyrazol-4-yl)pyrazolo[1,5-a]pyrazin-4-yl)cyclopentane-1-carboxylic acid (950 mg, 3.05 mmol, 1.00 eq.) in DCM (40 mL) was added DIEA (1.18 g, 9.15 mmol, 3.00 eq.) at 25° C. The mixture was stirred at 25° C. for 10 min. Then compound 7 (247.22 mg, 3.66 mmol, Hydrochloride, 1.20 eq.) and HATU (1.40 g, 3.66 mmol, 1.20 eq.) was added at 25° C. The reaction mixture was stirred at 25° C. for 1 hour. LCMS showed that the starting material was consumed completely and a major peak with desired MS was detected. The reaction mixture was concentrated to get a crude, which was purified by silica gel column chromatography eluting with EtOAc in PE from 0% to 100% to give rac-N-methyl-3-(6-(1-methyl-1H-pyrazol-4-yl)pyrazolo[1,5-a]pyrazin-4-yl)cyclopentane-1-carboxamide (900 mg, 90.93% yield) as yellow oil. LCMS: (M+H⁺: 325.2). ¹H NMR (400 MHz, METHANOL-d₄) δ=8.62 (s, 1H), 8.15-8.12 (m, 1H), 7.99-7.95 (m, 2H), 6.94-6.92 (m, 1H), 3.93 (s, 3H), 3.74-3.69 (m, 1H), 2.95 (s, 1H), 2.74-2.72 (m, 3H), 2.36-2.11 (m, 6H).

### 5. Preparation of rac-N-methyl-1-(3-(6-(1-methyl-1H-pyrazol-4-yl)pyrazolo[1,5-a]pyrazin-4-yl)cyclopentyl)methanamine

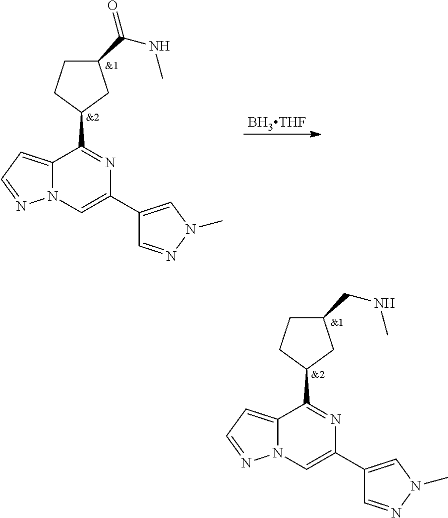 [SMILES: CNC(=O)[C@@H]1CC[C@H](c2nc(-c3cnn(C)c3)cn3nccc23)C1 ; "BH3·THF" ; CNC[C@@H]1CC[C@H](c2nc(-c3cnn(C)c3)cn3nccc23)C1]

To a solution of rac-N-methyl-3-(6-(1-methyl-H-pyrazol-4-yl)pyrazolo[1,5-a]pyrazin-4-yl)cyclopentane-1-carboxamide (200 mg, 616.56 mol, 1.00 eq.) in THF (10 mL) was added BH₃·THF (1 M, 3.08 mL, 5.00 eq.) at 0° C. The ice bath was removed and the reaction mixture was stirred at 35° C. for 2 hours. LCMS showed that the starting material was consumed completely and a major peak with desired MS was detected. The reaction mixture was quenched with MeOH (3 mL). The mixture was concentrated to give a crude rac-N-methyl-1-(3-(6-(1-methyl-1H-pyrazol-4-yl)pyrazolo[1,5-a]pyrazin-4-yl)cyclopentyl)methanamine (130.00 mg, crude), which was used to next step directly. LCMS: (M+H⁺: 311.2).

### 6. Preparation of rac-tert-butyl methyl((3-(6-(1-methyl-1H-pyrazol-4-yl)pyrazolo[1,5-a]pyrazin-4-yl)cyclopentyl)methyl)carbamate

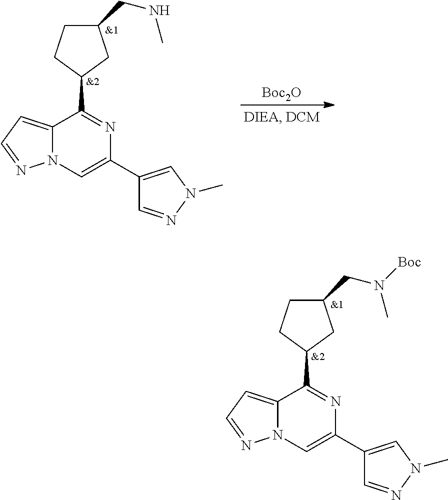 [SMILES: "Boc2O" ; CNC[C@@H]1CC[C@H](c2nc(-c3cnn(C)c3)cn3nccc23)C1 ; CN(C[C@@H]1CC[C@H](c2nc(-c3cnn(C)c3)cn3nccc23)C1)C(=O)OC(C)(C)C]

To the mixture of rac-N-methyl-1-(3-(6-(1-methyl-H-pyrazol-4-yl)pyrazolo[1,5-a]pyrazin-4-yl)cyclopentyl)methanamine (130 mg, 418.82 mol, 1.00 eq.) in MeOH (15 mL) was added DIEA (162.38 mg, 1.26 mmol, 3.00 eq.). The reaction mixture was stirred at 25° C. for 10 min. Then Boc₂O (137.11 mg, 628.23 mol, 1.50 eq.) was added at 25° C. The reaction mixture was stirred at 25° C. for 10 min. LCMS showed the starting material was consumed completely and a peak with desired MS was detected. The solvent was removed to get a crude, which was purified by silica gel column chromatography eluting with EtOAc in PE from 0% to 100% to give rac-tert-butyl methyl((3-(6-(1-methyl-1H-pyrazol-4-yl)pyrazolo[1,5-a]pyrazin-4-yl)cyclopentyl)methyl)carbamate (130 mg, 75.61% yield) as yellow oil. LCMS: (M+H⁺: 411.3). ¹H NMR: (400 MHz, METHANOL-d₄) δ=8.62 (s, 1H), 8.11 (s, 1H), 8.00-7.96 (m, 2H), 6.91 (s, 1H), 3.94-3.93 (m, 3H), 3.81 (s, 2H), 2.88 (s, 3H), 2.60-2.46 (m, 1H), 2.25-2.16 (m, 4H), 1.91-1.73 (m, 3H), 1.45-1.43 (m, 9H).

### 7. Preparation of rac-N-methyl-1-(3-(6-(1-methyl-1H-pyrazol-4-yl)pyrazolo[1,5-a]pyrazin-4-yl)cyclopentyl)methanamine hydrochloride

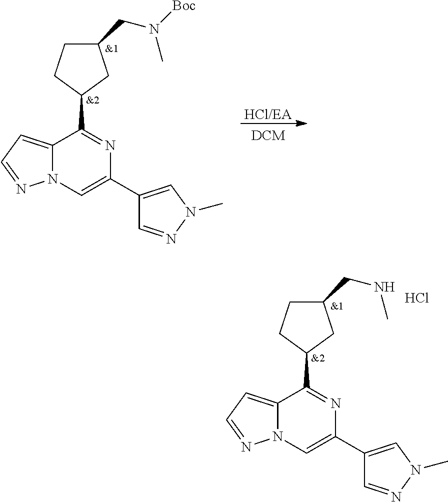 [SMILES: CN(C[C@@H]1CC[C@H](c2nc(-c3cnn(C)c3)cn3nccc23)C1)C(=O)OC(C)(C)C ; "HCl/EA" ; CNC[C@@H]1CC[C@H](c2nc(-c3cnn(C)c3)cn3nccc23)C1 ; Cl]

To a solution of rac-tert-butyl methyl((3-(6-(1-methyl-1H-pyrazol-4-yl)pyrazolo[1,5-a]pyrazin-4-yl)cyclopentyl)methyl)carbamate (280 mg, 682.08 mol, 1.0 eq.) in DCM (10 mL) was added HCl/EA (4 M, 10 mL) at 20° C. and the reaction was stirred at 20° C. for 30 min. LCMS showed the starting material was consumed completely and a peak with desired MS was detected. The mixture was concentrated under vacuum to give rac-N-methyl-1-(3-(6-(1-methyl-1H-pyrazol-4-yl)pyrazolo[1,5-a]pyrazin-4-yl)cyclopentyl)methanamine hydrochloride (220 mg, crude) as yellow oil. LCMS: (M+H⁺: 311.3).

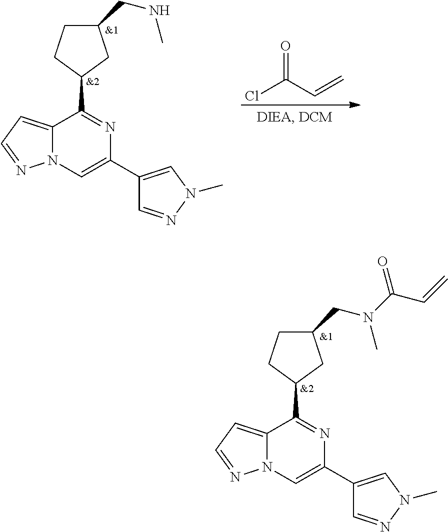

### 8. Preparation of rac-N-methyl-N-((3-(6-(1-methyl-1H-pyrazol-4-yl)pyrazolo[1,5-a]pyrazin-4-yl)cyclopentyl)methyl)acrylamide

To the mixture of rac-N-methyl-1-(3-(6-(1-methyl-H-pyrazol-4-yl)pyrazolo[1,5-a]pyrazin-4-yl)cyclopentyl)methanamine hydrochloride (220 mg, 708.77 mol, 1.0 eq.) and DIPEA (183.20 mg, 1.42 mmol, 2.0 eq.) in DCM (20 mL) was added compound 12 (70.56 mg, 779.65 mol, 1.1 eq.) at 0° C. The mixture was stirred at 0° C. for 10 min. LCMS showed the starting material was consumed completely. MeOH (3 mL) was added dropwise. The reaction mixture was concentrated to give rac-N-methyl-N-((3-(6-(1-methyl-1H-pyrazol-4-yl)pyrazolo[1,5-a]pyrazin-4-yl)cyclopentyl)methyl)acrylamide (220 mg, crude) as yellow oil. LCMS: (M+H⁺: 365.3.).

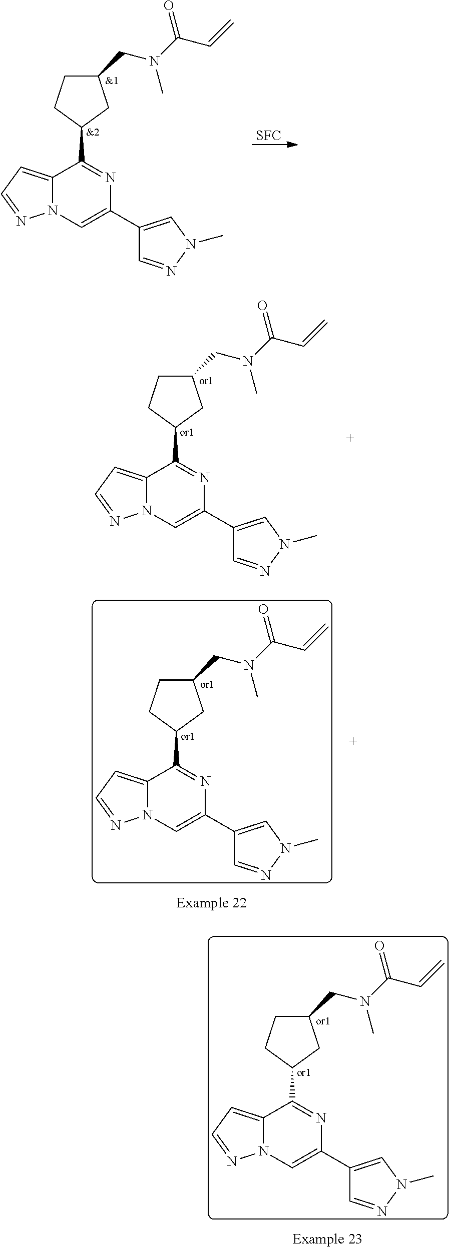

### 9. General procedure for separation of rac-N-methyl-N-((3-(6-(1-methyl-1H-pyrazol-4-yl)pyrazolo[1,5-a]pyrazin-4-yl)cyclopentyl)methyl)acrylamide

rac-N-methyl-N-((3-(6-(1-methyl-H-pyrazol-4-yl)pyrazolo[1,5-a]pyrazin-4-yl)cyclopentyl)methyl)acrylamide (220.00 mg, 603.66 mol, 1.0 eq.) was separated by SFC (Column: DAICEL CHIRALPAK AD (250 mm×30 mm, 10 pm); Condition: 0.1% NH₃H₂O ETOH, Begin B 45%, End B 45%, Flow Rate (mL/min) 70.) to give N-methyl-N-((3-(6-(1-methyl-1H-pyrazol-4-yl)pyrazolo[1,5-a]pyrazin-4-yl)cyclopentyl)methyl)acrylamide as a mixture of two isomers (100 mg, 45.45% yield) as a yellow solid, Example 22 (45.8 mg, 19.68% yield) as a yellow solid and Example 23 (42.7 mg, 18.85% yield) as a yellow solid.

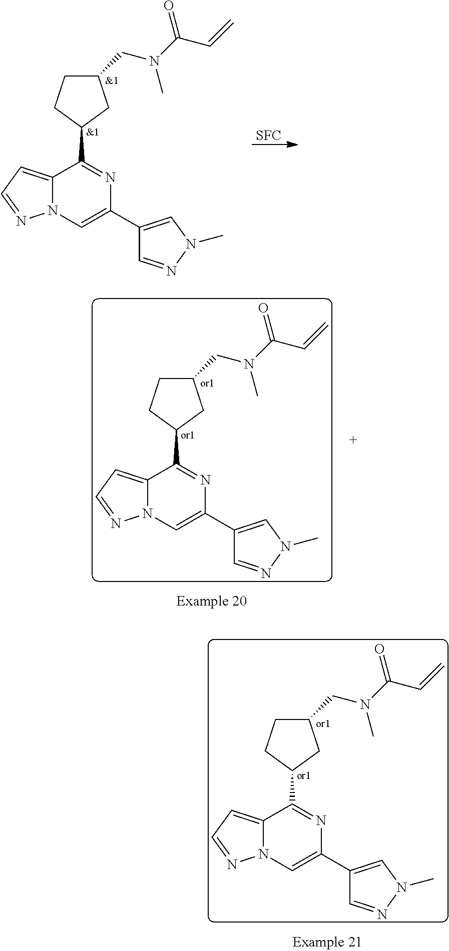 [SMILES: C=CC(=O)N(C)C[C@H]1CC[C@H](c2nc(-c3cnn(C)c3)cn3nccc23)C1 ; "SFC" ; C=CC(=O)N(C)C[C@H]1CC[C@H](c2nc(-c3cnn(C)c3)cn3nccc23)C1 ; C=CC(=O)N(C)C[C@H]1CC[C@@H](c2nc(-c3cnn(C)c3)cn3nccc23)C1]

### 10. General procedure for separation of rac-N-methyl-N-((3-(6-(1-methyl-1H-pyrazol-4-yl)pyrazolo[1,5-a]pyrazin-4-yl)cyclopentyl)methyl)acrylamide

N-methyl-N-((3-(6-(1-methyl-1H-pyrazol-4-yl)pyrazolo[1,5-a]pyrazin-4-yl)cyclopentyl)methyl)acrylamide as a mixture of two isomers (100.00 mg, 274.39 μmol, 1.0 eq.) was separated by SFC (Column: Phenomenex-Cellulose-2 (250 mm×30 mm, 10 pm); Condition: 0.1% NH₃H₂O ETOH, Begin B 45, End B 45, Flow Rate (mL/min) 80.) to give Example 20 (33.4 mg, 33.40% yield) as a yellow solid and Example 21 (35.8 mg, 35.80% yield) as a yellow solid.

### Spectra of Example 20

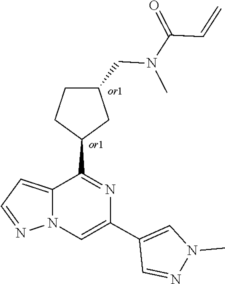 [SMILES: C=CC(=O)N(C)C[C@H]1CC[C@H](c2nc(-c3cnn(C)c3)cn3nccc23)C1]

LCMS: (M+H⁺: 365.2). HPLC: (Purity: 100.00%). SFC: (ee: 100.00%). ¹H NMR: (400 MHz, METHANOL-d₄) δ=8.62 (d, J=4.4 Hz, 1H), 8.11 (d, J=8.4 Hz, 1H), 8.04-7.91 (m, 2H), 6.96-6.72 (m, 2H), 6.31-6.19 (m, 1H), 5.80-5.69 (m, 1H), 3.95 (s, 3H), 3.89-3.80 (m, 1H), 3.71-3.39 (m, 2H), 3.18-3.03 (m, 3H), 2.66-2.51 (m, 1H), 2.38-2.00 (m, 4H), 1.91-1.80 (m, 1H), 1.59-1.43 (m, 1H).

### Spectra of Example 21

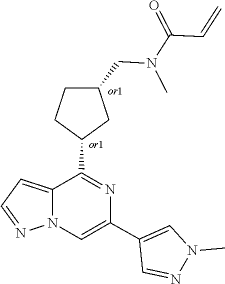 [SMILES: C=CC(=O)N(C)C[C@H]1CC[C@@H](c2nc(-c3cnn(C)c3)cn3nccc23)C1]

LCMS: (M+H⁺: 365.2). HPLC: (Purity: 100.00%). SFC: (ee: 100.00%). ¹H NMR: (500 MHz, METHANOL-d₄) δ=8.84 (d, J=3.5 Hz, 1H), 0.42-8.16 (m, 3H), 7.16-6.89 (m, 2H), 6.43-6.31 (m, 1H), 5.96-5.74 (m, 1H), 4.17-4.09 (m, 3H), 4.00-3.69 (m, 3H), 3.36-3.17 (m, 3H), 2.77-2.67 (m, 1H), 2.48-2.32 (m, 3H), 2.16-2.04 (m, 2H), 1.88-1.73 (m, 1H).

### Spectra of Example 22

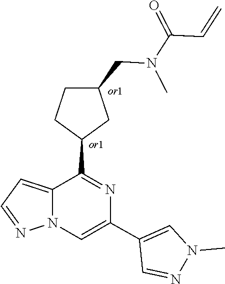 [SMILES: C=CC(=O)N(C)C[C@@H]1CC[C@H](c2nc(-c3cnn(C)c3)cn3nccc23)C1]

LCMS: (M+H⁺: 365.2). HPLC: (Purity: 94.52%). SFC: (ee: 99.04%). ¹H NMR: (400 MHz, METHANOL-d₄) δ=8.64 (d, J=6.4 Hz, 1H), 8.24-7.91 (m, 3H), 6.95-6.70 (m, 2H), 6.23-6.16 (m, 1H), 5.76-5.58 (m, 1H), 3.96 (s, 3H), 3.82-3.50 (m, 3H), 3.21-2.96 (m, 3H), 2.61-2.50 (m, 1H), 2.34-2.09 (m, 3H), 2.00-1.83 (m, 2H), 1.69-1.57 (m, 1H).

### Spectra of Example 23

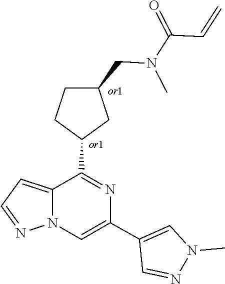 [SMILES: C=CC(=O)N(C)C[C@@H]1CC[C@@H](c2nc(-c3cnn(C)c3)cn3nccc23)C1]

LCMS: (M+H⁺: 365.2). HPLC: (Purity: 97.13%). SFC: (ee: 100.00%). ¹H NMR: (400 MHz, METHANOL-d₄) δ=8.62 (d, J=4.4 Hz, 1H), 8.11 (d, J=8.4 Hz, 1H), 8.06-7.83 (m, 2H), 6.98-6.69 (m, 2H), 6.31-6.18 (m, 1H), 5.77-5.72 (m, 1H), 3.94 (s, 3H), 3.88-3.79 (m, 1H), 3.69-3.39 (m, 2H), 3.21-2.94 (m, 3H), 2.72-2.58 (m, 1H), 2.37-2.01 (m, 4H), 1.91-1.76 (m, 1H), 1.57-1.45 (m, 1H).

### Examples 24-27

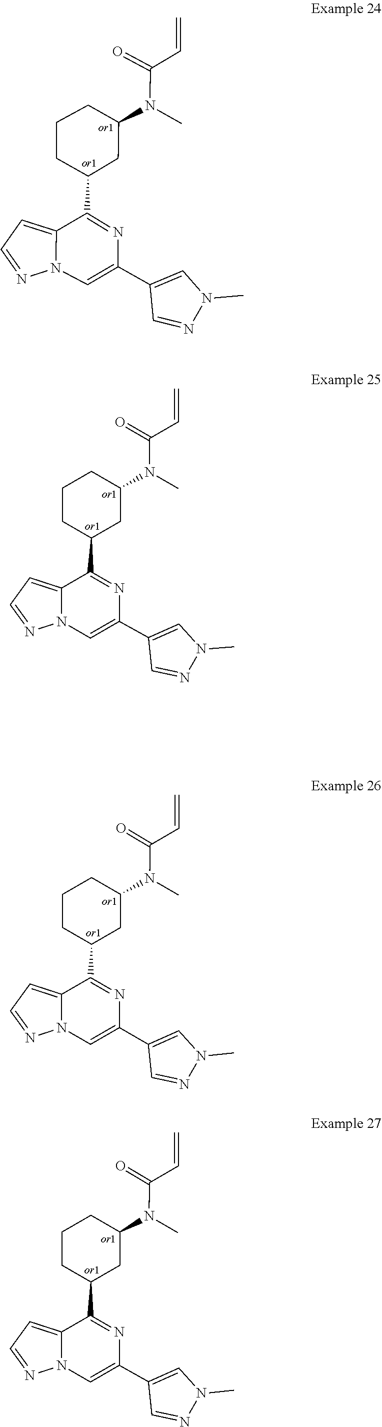 [SMILES: C=CC(=O)N(C)[C@@H]1CCC[C@@H](c2nc(-c3cnn(C)c3)cn3nccc23)C1 ; C=CC(=O)N(C)[C@H]1CCC[C@H](c2nc(-c3cnn(C)c3)cn3nccc23)C1 ; C=CC(=O)N(C)[C@H]1CCC[C@@H](c2nc(-c3cnn(C)c3)cn3nccc23)C1 ; C=CC(=O)N(C)[C@@H]1CCC[C@H](c2nc(-c3cnn(C)c3)cn3nccc23)C1]

### 1. Preparation of rac-tert-butyl (3-(6-(1-methyl-1H-pyrazol-4-yl)pyrazolo[1,5-a]pyrazin-4-yl)cyclohex-2-en-1-yl)carbamate

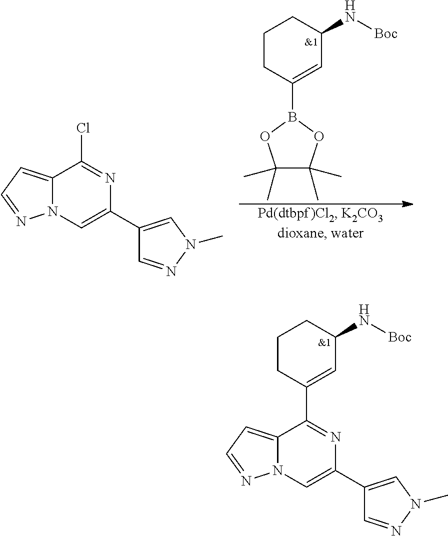

To a mixture of 4-chloro-6-(1-methylpyrazol-4-yl)pyrazolo[1,5-a]pyrazine (300.00 mg, 1.28 mmol, 1.0 eq.), rac-tert-butyl (R)-(3-(4,4,5,5-tetramethyl-1,3,2-dioxaborolan-2-yl)cyclohex-2-en-1-yl)carbamate (700.00 mg, 1.30 mmol, 1.0 eq.), K₂CO₃ (532.36 mg, 3.85 mmol, 3.0 eq.), Pd(dtbpf)Cl₂ (83.68 mg, 128.39 mol, 0.1 eq.) in dioxane (10 mL)/water (2 mL) was bubbled with N₂ for 1 min and the reaction was stirred at 90° C. for 2 h. LCMS showed the starting material was consumed completely. The reaction mixture was concentrated to get a crude, which was purified by silica gel column chromatography (PE to PE/EA=1/1 to EA) to give rac-tert-butyl (R)-(3-(6-(1-methyl-1H-pyrazol-4-yl)pyrazolo[1,5-a]pyrazin-4-yl)cyclohex-2-en-1-yl)carbamate (350.00 mg, 62.19% yield) as a yellow solid. LCMS: (M+H⁺: 395.7).

### 2. Preparation of rac-tert-butyl ((1R,3S)-3-(6-(1-methyl-1H-pyrazol-4-yl)pyrazolo[1,5-a]pyrazin-4-yl)cyclohexyl)carbamate

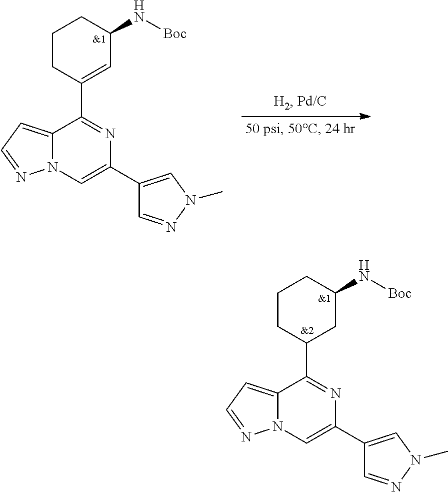 [SMILES: Cn1cc(-c2cn3nccc3c(C3=C[C@H](NC(=O)OC(C)(C)C)CCC3)n2)cn1 ; Cn1cc(-c2cn3nccc3c(C3CCC[C@@H](NC(=O)OC(C)(C)C)C3)n2)cn1]

The mixture of rac-tert-butyl (R)-(3-(6-(1-methyl-H-pyrazol-4-yl)pyrazolo[1,5-a]pyrazin-4-yl)cyclohex-2-en-1-yl)carbamate (610.00 mg, 1.55 mmol, 1.0 eq.) in MeOH (30 mL) was added Pd/C (700.00 mg, 6.58 mmol) at 15° C. The reaction was stirred at 50° C. for 24 h under H₂ (50 Psi). LCMS showed the starting material was consumed completely and a peak with desired MS was detected. The mixture was filtered to remove Pd/C and concentrated under vacuum to removed MeOH for giving rac-tert-butyl ((1R,3S)-3-(6-(1-methyl-1H-pyrazol-4-yl)pyrazolo[1,5-a]pyrazin-4-yl)cyclohexyl)carbamate (550.00 mg, crude) as yellow oil. LCMS: (M+H⁺: 397.2).

### 3. Preparation of rac-tert-butyl methyl(3-(6-(1-methyl-1H-pyrazol-4-yl)pyrazolo[1,5-a]pyrazin-4-yl)cyclohexyl)carbamate

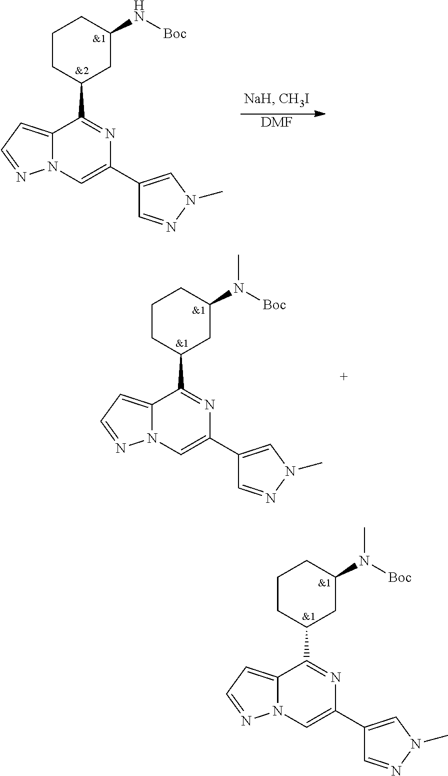

To a solution of rac-tert-butyl ((1R,3S)-3-(6-(1-methyl-1H-pyrazol-4-yl)pyrazolo[1,5-a]pyrazin-4-yl)cyclohexyl)carbamate (600.00 mg, 1.51 mmol, 1.0 eq.) in DMF (10 mL) was added NaH (181.60 mg, 4.54 mmol, 60% purity, 3.0 eq.) at 0° C. The reaction mixture was stirred at 0° C. for 30 min. Then CH₃I (644.39 mg, 4.54 mmol, 3.0 eq.) was added to the mixture at 0° C. The reaction mixture was stirred at 20° C. for 6 hours. LCMS showed the desired MS was detected and the starting material was consumed completely. The reaction mixture was quenched with MeOH (5 mL). The reaction mixture was removed to give the crude, which was purified by prep HPLC (Column: Waters Xbridge BEH C18 100×25 mm×5 μm; Condition: water (0.225% FA)-ACN, Begin B 38, End B 68, Gradient Time (min) 12, 100% B Hold Time (min) 2, Flow Rate (mL/min) 25.) to give rac-cis-tert-butyl methyl(3-(6-(1-methyl-1H-pyrazol-4-yl)pyrazolo[1,5-a]pyrazin-4-yl)cyclohexyl)carbamate (200 mg, 26.40% yield) as a white solid, rac-trans-tert-butyl methyl(3-(6-(1-methyl-1H-pyrazol-4-yl)pyrazolo[1,5-a]pyrazin-4-yl)cyclohexyl)carbamate (150 mg, 22.94% yield) as a white solid.

rac-cis-tert-butyl methyl(3-(6-(1-methyl-H-pyrazol-4-yl)pyrazolo[1,5-a]pyrazin-4-yl)cyclohexyl)carbamate: LCMS: (M+H⁺: 411.8).

rac-trans-tert-butyl methyl(3-(6-(1-methyl-H-pyrazol-4-yl)pyrazolo[1,5-a]pyrazin-4-yl)cyclohexyl)carbamate: LCMS: (M+H⁺: 411.8.).

### 4. Preparation of rac-trans-N-methyl-3-(6-(1-methyl-1H-pyrazol-4-yl)pyrazolo[1,5-a]pyrazin-4-yl)cyclohexan-1-amine hydrochloride

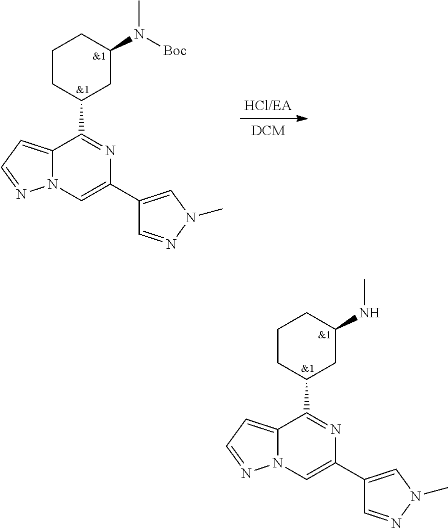 [SMILES: CN(C(=O)OC(C)(C)C)[C@@H]1CCC[C@@H](c2nc(-c3cnn(C)c3)cn3nccc23)C1 ; CN[C@@H]1CCC[C@@H](c2nc(-c3cnn(C)c3)cn3nccc23)C1]

To a solution of rac-trans-tert-butyl methyl(3-(6-(1-methyl-1H-pyrazol-4-yl)pyrazolo[1,5-a]pyrazin-4-yl)cyclohexyl)carbamate (150.00 mg, 365.40 mol, 1.0 eq.) in DCM (2 mL) was added HCl/EA (4 M, 15.00 mL) at 20° C. and the reaction was stirred at 20° C. for 30 min. LCMS showed the starting material was consumed completely and a peak with desired MS was detected. The mixture was concentrated under vacuum to give rac-trans-N-methyl-3-(6-(1-methyl-1H-pyrazol-4-yl)pyrazolo[1,5-a]pyrazin-4-yl)cyclohexan-1-amine hydrochloride (90.00 mg, crude) as yellow oil. LCMS: (M+H⁺: 311.3).

### 5. Preparation of rac-trans-N-methyl-N-(3-(6-(1-methyl-1H-pyrazol-4-yl)pyrazolo[1,5-a]pyrazin-4-yl)cyclohexyl)acrylamide

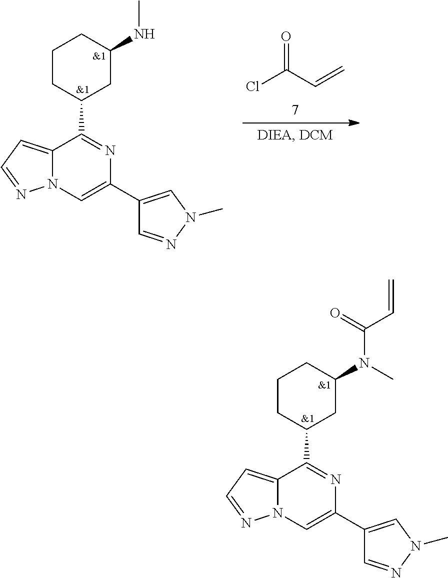

To the mixture of rac-trans-N-methyl-3-(6-(1-methyl-H-pyrazol-4-yl)pyrazolo[1,5-a]pyrazin-4-yl)cyclohexan-1-amine hydrochloride (90.00 mg, 289.95 mol, 1.0 eq.) and DIPEA (74.95 mg, 579.90 mol, 2.0 eq.) in DCM (6 mL) was added compound 7 (30 mg, 331.46 mol, 1.14 eq.) at 0° C. The mixture was stirred at 0° C. for 10 min. LCMS showed the starting material was consumed completely. MeOH (3 mL) was added dropwise. The reaction mixture was concentrated to give rac-trans-N-methyl-N-(3-(6-(1-methyl-1H-pyrazol-4-yl)pyrazolo[1,5-a]pyrazin-4-yl)cyclohexyl)acrylamide (100.00 mg, crude) as yellow oil. LCMS: (M+H⁺: 365.2.).

### 6. Separation of rac-trans-N-methyl-N-(3-(6-(1-methyl-1H-pyrazol-4-yl)pyrazolo[1,5-a]pyrazin-4-yl)cyclohexyl)acrylamide

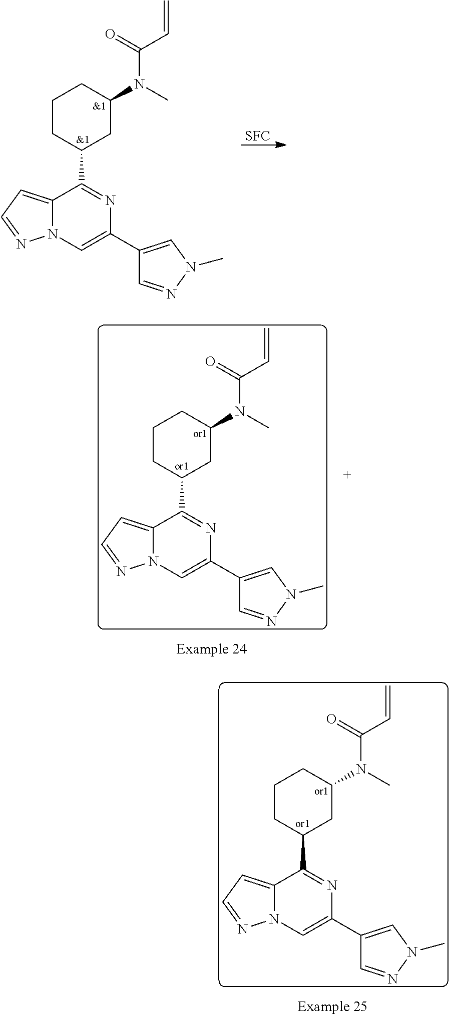 [SMILES: C=CC(=O)N(C)[C@@H]1CCC[C@@H](c2nc(-c3cnn(C)c3)cn3nccc23)C1 ; "SFC" ; C=CC(=O)N(C)[C@@H]1CCC[C@@H](c2nc(-c3cnn(C)c3)cn3nccc23)C1 ; C=CC(=O)N(C)[C@H]1CCC[C@H](c2nc(-c3cnn(C)c3)cn3nccc23)C1]

rac-trans-N-methyl-N-(3-(6-(1-methyl-H-pyrazol-4-yl)pyrazolo[1,5-a]pyrazin-4-yl)cyclohexyl)acrylamide (100.00 mg, 274.39 mol, 1.0 eq.) was separated by SFC (Column: Phenomenex-Cellulose-2 (250 mm×30 mm, 110 μm); Condition: 0.1% NH₃H₂O ETOH, Begin B 40, End B 40, Flow Rate (mL/min) 80.) to give Example 24 (41.1 mg, 40.15% yield) as a yellow solid and Example 25 (47.1 mg, 47.10% yield) as a yellow solid.

### Spectra of Example 24

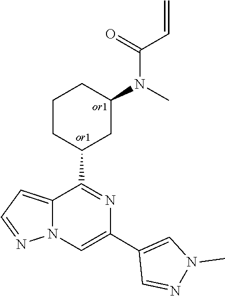 [SMILES: C=CC(=O)N(C)[C@@H]1CCC[C@@H](c2nc(-c3cnn(C)c3)cn3nccc23)C1]

LCMS (M+H⁺: 365.2). HPLC: (Purity: 97.70%). SFC (Purity: 94.99%). ¹H NMR: (400 MHz, METHANOL-d₄) δ=8.68-8.62 (m, 1H), 8.13-7.92 (m, 2H), 6.95-6.68 (m, 2H), 6.28-6.12 (m, 1H), 5.84-5.70 (m, 1H), 5.46-5.05 (m, 1H), 4.17-3.75 (m, 4H), 3.11-2.82 (m, 3H), 2.40-1.41 (m, 9H).

### Spectra of Example 25

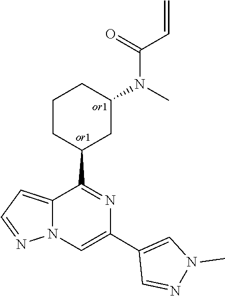 [SMILES: C=CC(=O)N(C)[C@H]1CCC[C@H](c2nc(-c3cnn(C)c3)cn3nccc23)C1]

LCMS: (M+H⁺: 365.3). HPLC: (Purity: 100.00%). SFC: (Purity: 99.52%). ¹H NMR: (400 MHz, METHANOL-d₄) δ=8.71-8.61 (m, 1H), 8.16-7.95 (m, 2H), 6.98-6.70 (m, 2H), 6.28-6.14 (m, 1H), 5.84-5.70 (m, 1H), 5.46-5.05 (m, 1H), 4.11-3.72 (m, 4H), 3.13-2.86 (m, 3H), 2.33-1.49 (m, 9H).

### 7. Preparation of rac-cis-N-methyl-3-(6-(1-methyl-1H-pyrazol-4-yl)pyrazolo[1,5-a]pyrazin-4-yl)cyclohexan-1-amine hydrochloride

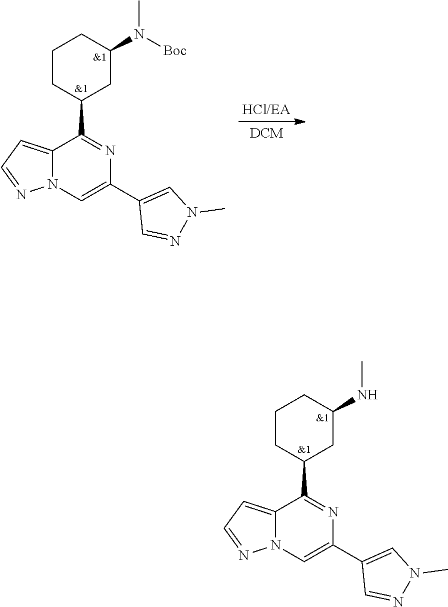 [SMILES: CN(C(=O)OC(C)(C)C)[C@@H]1CCC[C@H](c2nc(-c3cnn(C)c3)cn3nccc23)C1 ; CN[C@@H]1CCC[C@H](c2nc(-c3cnn(C)c3)cn3nccc23)C1]

The mixture of rac-cis-tert-butyl methyl(3-(6-(1-methyl-1H-pyrazol-4-yl)pyrazolo[1,5-a]pyrazin-4-yl)cyclohexyl)carbamate (200 mg, 0.487 mmol, 1.0 eq.) in DCM (2 mL) was added HCl in EtOAc (4 M, 8.00 mL). The mixture was stirred at 25° C. for 1 hour. LCMS showed the starting material was consumed completely and a peak with desired MS was detected. The reaction mixture was concentrated to give rac-cis-N-methyl-3-(6-(1-methyl-1H-pyrazol-4-yl)pyrazolo[1,5-a]pyrazin-4-yl)cyclohexan-1-amine hydrochloride (120 mg, crude) as a yellow solid, which was used to next step directly. LCMS: (M+H⁺: 311.2).

### 8. Preparation of rac-cis-N-methyl-N-(3-(6-(1-methyl-1H-pyrazol-4-yl)pyrazolo[1,5-a]pyrazin-4-yl)cyclohexyl)acrylamide

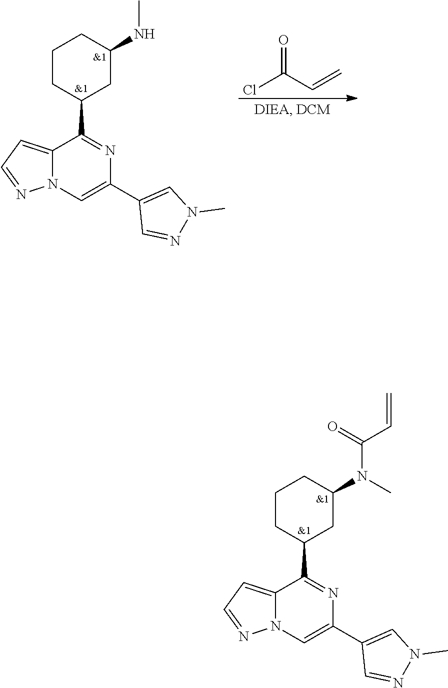

To the mixture of rac-cis-N-methyl-3-(6-(1-methyl-H-pyrazol-4-yl)pyrazolo[1,5-a]pyrazin-4-yl)cyclohexan-1-amine hydrochloride (120 mg, 0.386 mmol, 1.0 eq.) and DIPEA (149.90 mg, 1.16 mmol, 3.0 eq.) in DCM (10 mL) was added acryloyl chloride (38.49 mg, 0.425 mmol, 1.0 eq.) at 0° C. The mixture was stirred at 0° C. for 2 min. LCMS showed the starting material was consumed completely and a peak with desired MS was detected. MeOH (1 mL) was added dropwise. The resulting mixture was stirred at 25° C. for 10 min. The solvent was removed to get a crude rac-cis-N-methyl-N-(3-(6-(1-methyl-1H-pyrazol-4-yl)pyrazolo[1,5-a]pyrazin-4-yl)cyclohexyl)acrylamide (102 mg, crude) as a yellow oil. LCMS: (M+H⁺: 365.2).

### 9. Separation of cis-N-methyl-N-(3-(6-(1-methyl-1H-pyrazol-4-yl)pyrazolo[1,5-a]pyrazin-4-yl)cyclohexyl)acrylamide

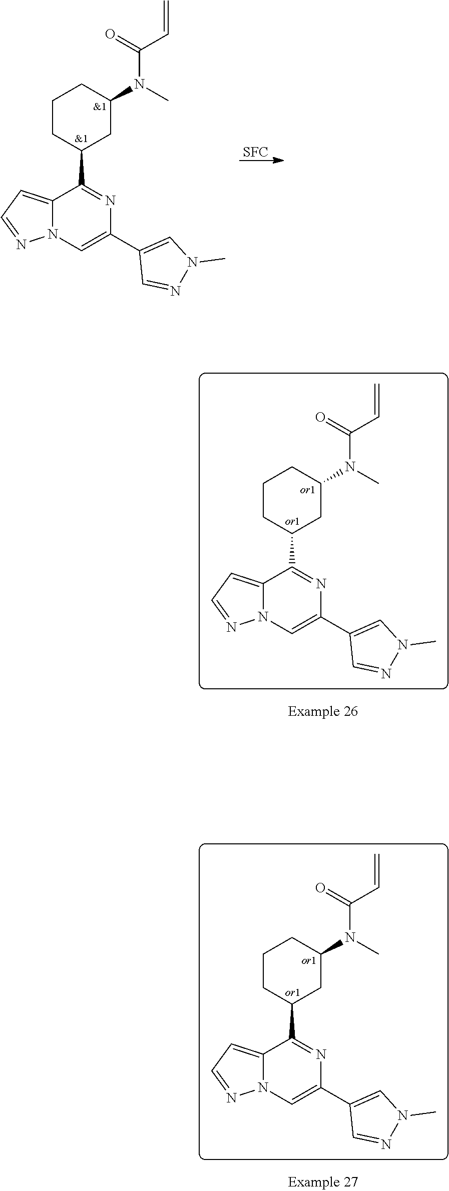 [SMILES: C=CC(=O)N(C)[C@@H]1CCC[C@H](c2nc(-c3cnn(C)c3)cn3nccc23)C1 ; "SFC" ; C=CC(=O)N(C)[C@H]1CCC[C@@H](c2nc(-c3cnn(C)c3)cn3nccc23)C1 ; C=CC(=O)N(C)[C@@H]1CCC[C@H](c2nc(-c3cnn(C)c3)cn3nccc23)C1]

rac-cis-N-methyl-N-(3-(6-(1-methyl-H-pyrazol-4-yl)pyrazolo[1,5-a]pyrazin-4-yl)cyclohexyl)acrylamide (102 mg, 0.280 mmol, 1.0 eq.) was separated by SFC (Column: DAICEL CHIRALPAK IG (250 mm×30 mm, 10 μm); Condition: 0.1% NH₃H₂O EtOH, Begin B 50%, End B 50%, Gradient Time (min), 100% B Hold Time (min), Flow Rate (mL/min) 80) to give Example 26 (37.2 mg, 36.47% yield) as a white solid and Example 27 (33.4 mg, 32.75% yield,) as a pale yellow solid.

### Spectra of Example 26

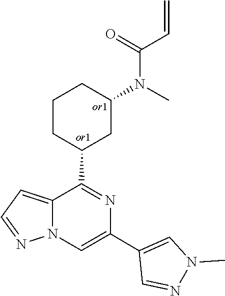 [SMILES: C=CC(=O)N(C)[C@H]1CCC[C@@H](c2nc(-c3cnn(C)c3)cn3nccc23)C1]

LCMS: (M+H⁺: 365.2). SFC: (ee: 99.57%). HPLC: (Purity: 100%). ¹H NMR: (400 MHz, METHANOL-d₄) δ=8.64 (d, J=4.0 Hz, 1H), 8.14 (s, 1H), 8.05-7.92 (m, 2H), 6.99 (s, 1H), 6.93-6.66 (m, 1H), 6.23-6.16 (m, 1H), 5.74 (d, J=10.8 Hz, 1H), 4.41-4.39 (m, 1H), 3.95 (s, 3H), 3.41-3.38 (m, 1H), 3.09-2.91 (m, 3H), 2.32-1.89 (m, 4H), 1.86-1.61 (m, 4H).

### Spectra of Example 27

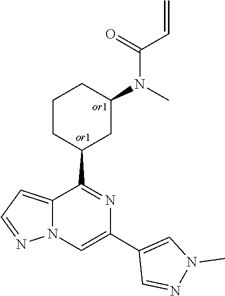 [SMILES: C=CC(=O)N(C)[C@@H]1CCC[C@H](c2nc(-c3cnn(C)c3)cn3nccc23)C1]

LCMS: (M+H⁺: 365.3). SFC: (ee: 98.44%). HPLC: (Purity: 100%). ¹H NMR: (400 MHz, METHANOL-d₄) δ=8.65 (d, J=4.0 Hz, 1H), 8.15 (s, 1H), 8.05-7.92 (m, 2H), 6.99 (s, 1H), 6.93-6.66 (m, 1H), 6.23-6.16 (m, 1H), 5.74 (d, J=10.8 Hz, 1H), 4.41-4.39 (m, 1H), 3.95 (s, 3H), 3.41-3.38 (m, 1H), 3.09-2.91 (m, 3H), 2.32-1.89 (m, 4H), 1.86-1.61 (m, 4H).

### Example 28. N-(3-(6-(1-methyl-1H-pyrazol-4-yl)pyrazolo[1,5-a]pyrazin-4-yl)phenyl) acrylamide

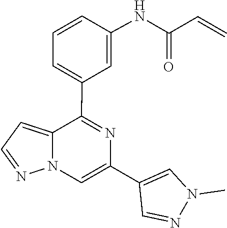 [SMILES: C=CC(=O)Nc1cccc(-c2nc(-c3cnn(C)c3)cn3nccc23)c1]

### 1. Preparation of 6-(1-methyl-1H-pyrazol-4-yl)-4-(3-nitrophenyl)pyrazolo[1,5-a]pyrazine

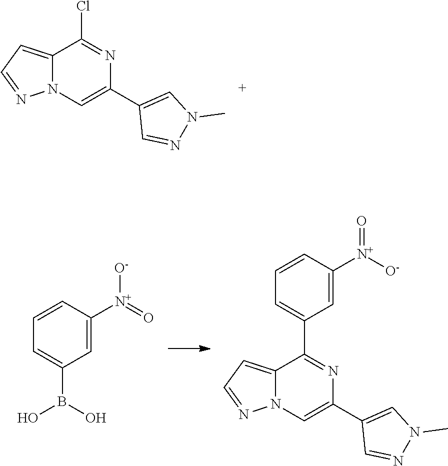 [SMILES: Cn1cc(-c2cn3nccc3c(Cl)n2)cn1 ; O=[N+]([O-])c1cccc(B(O)O)c1 ; Cn1cc(-c2cn3nccc3c(-c3cccc([N+](=O)[O-])c3)n2)cn1]

To an 8 mL vial was added 4-chloro-6-(1-methylpyrazol-4-yl)pyrazolo[1,5-a]pyrazine (150 mg, 641.97 umol), (3-nitrophenyl)boronic acid (215 mg, 1.29 mmol), Pd(dppf)Cl₂ (15 mg, 20.50 umol), K₂CO₃ (250 mg, 1.81 mmol), and EtOH (3 mL) which was sealed and heated to 85° C. for 2 hours, then analyzed by LCMS which showed complete consumption of the starting material. The reaction was cooled to room temperature.

The reaction mixture was diluted with 5 mL EtOH and stirred at 40° C. for 30 minutes, then filtered via 0.2 um syringe filter. The filtrate was analyzed by LCMS which showed only trace presence of desired product. The EtOH filtrate was discarded. The solids remaining 10 mL DCM which completely dissolved all material. The DCM filtrate was concentrated to give the desired product as a tan solid, 6-(1-methylpyrazol-4-yl)-4-(3-nitrophenyl)pyrazolo[1,5-a]pyrazine (300 mg, 936.61 umol, 145.90% yield). LCMS: (M+H⁺: 321.1). ¹H NMR (500 MHz, DMSO-d₆) δ ppm 8.86 (t, J=1.83 Hz, 1H) 8.59 (dd, J=7.94, 1.22 Hz, 1H) 8.42-8.47 (m, 1H) 8.37 (s, 1H) 8.27 (d, J=2.44 Hz, 1H) 8.14 (s, 1H) 7.92 (s, 1H) 7.25 (d, J=2.44 Hz, 1H) 3.92 (s, 3H).

### 2. Preparation of 3-(6-(1-methyl-1H-pyrazol-4-yl)pyrazolo[1,5-a]pyrazin-4-yl)aniline

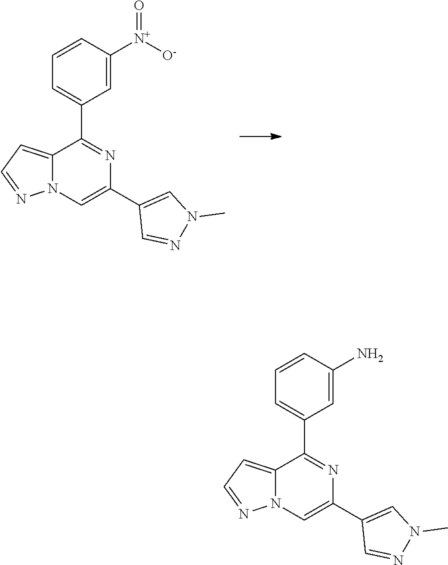 [SMILES: Cn1cc(-c2cn3nccc3c(-c3cccc([N+](=O)[O-])c3)n2)cn1 ; Cn1cc(-c2cn3nccc3c(-c3cccc(N)c3)n2)cn1]

To a 30 mL vial containing 6-(1-methylpyrazol-4-yl)-4-(3-nitrophenyl)pyrazolo[1,5-a]pyrazine (200 mg, 624.40 umol), was added Pd(OH)₂/C (125 mg, 178.02 umol, 20% purity) followed by 3:1 EtOAc:EtOH (8 mL). This mixture was purged with H₂ for 15 minutes, then left to stir under a balloon of H₂ for 1 hour at 40° C. The reaction was analyzed by LCMS which showed complete conversion to the desired product. The H₂ balloon was removed.

The reaction was filtered through celite, rinsing with 25 mL 3:1 EtOAc:EtOH. The filtrate was concentrated to dryness to give a light yellow solid, 3-[6-(1-methylpyrazol-4-yl)pyrazolo[1,5-a]pyrazin-4-yl]aniline (175 mg, 602.78 umol, 96.54% yield). LCMS: (M+H⁺: 291.1). ¹H NMR (500 MHz, DMSO-d₆) δ ppm 9.09 (d, J=1.22 Hz, 1H) 8.31 (s, 1H) 8.17 (d, J=2.44 Hz, 1H) 8.10 (s, 1H) 7.36 (t, J=1.83 Hz, 1H) 7.24 (d, J=14.04 Hz, 2H) 7.09 (dd, J=2.44, 1.22 Hz, 1H) 6.74-6.79 (m, 1H) 5.35 (s, 2H) 3.91 (s, 3H).

### 3. Preparation of N-(3-(6-(1-methyl-H-pyrazol-4-yl)pyrazolo[1,5-a]pyrazin-4-yl)phenyl)acrylamide

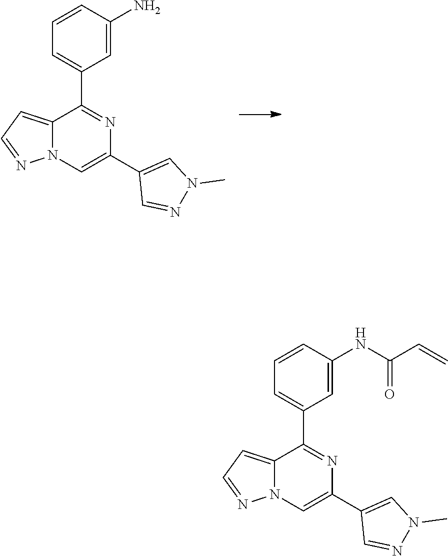 [SMILES: Cn1cc(-c2cn3nccc3c(-c3cccc(N)c3)n2)cn1 ; C=CC(=O)Nc1cccc(-c2nc(-c3cnn(C)c3)cn3nccc23)c1]

To a 30 mL vial containing 3-[6-(1-methylpyrazol-4-yl)pyrazolo[1,5-a]pyrazin-4-yl]aniline (175 mg, 602.78 umol) was added DCM (5 mL) and DIPEA (148.40 mg, 1.15 mmol, 200 uL) which was stirred at room temperature for 5 minutes. The mixture was cooled on a dry ice/acetone bath for 10 minutes, then Acryloyl chloride (54.56 mg, 602.78 umol, 48.97 uL) was added. The reaction was warmed to room temperature and stirred an additional 15 minutes.

The reaction was purified by silica gel column chromatography (12 g, heptane to 3:1 EtOAc:EtOH). The desired fractions were collected and concentrated to give a white solid, N-[3-[6-(1-methylpyrazol-4-yl)pyrazolo[1,5-a]pyrazin-4-yl]phenyl]prop-2-enamide (180 mg, 512.24 umol, 84.98% yield, 98% purity). LCMS: (M+H⁺: 345.1). ¹H NMR (600 MHz, DMSO-d₆) δ ppm 10.39 (s, 1H) 9.15 (s, 1H) 8.50-8.59 (m, 1H) 8.30-8.37 (m, 1H) 8.19-8.26 (m, 1H) 8.09-8.14 (m, 1H) 7.81-7.93 (m, 2H) 7.52-7.64 (m, 1H) 7.15-7.24 (m, 1H) 6.44-6.54 (m, 1H) 6.29-6.39 (m, 1H) 5.76-5.87 (m, 1H) 3.93 (s, 3H).

### Example 29. N-methyl-N-(3-(6-(1-methyl-1H-pyrazol-4-yl)pyrazolo[1,5-a]pyrazin-4-yl)phenyl)acrylamide

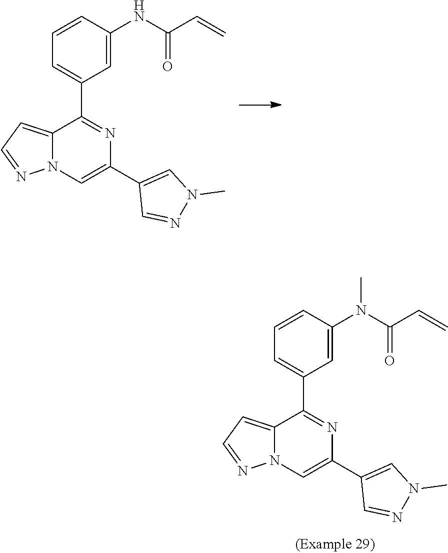 [SMILES: C=CC(=O)Nc1cccc(-c2nc(-c3cnn(C)c3)cn3nccc23)c1 ; C=CC(=O)N(C)c1cccc(-c2nc(-c3cnn(C)c3)cn3nccc23)c1]

To a 30 mL vial containing N-[3-[6-(1-methylpyrazol-4-yl)pyrazolo[1,5-a]pyrazin-4-yl]phenyl]prop-2-enamide (130 mg, 377.50 umol) was added THF (5 mL) and Iodomethane (130 mg, 915.89 umol, 57.02 uL) which was stirred at room temperature for 5 minutes, then Potassium tert-butoxide (1 M, 750 uL) was added dropwise. The reaction was allowed to stir for 15 minutes, then analyzed by LCMS which showed complete conversion to the desired product. The reaction was diluted with 10 mL water and extracted 3×10 mL EtOAc. The combined organic layers were concentrated to dryness, then purified by silica gel column chromatography (12 g, heptane to 3:1 EtOAc:EtOH). The desired fractions were collected and concentrated to give a yellow foam solid, N-methyl-N-[3-[6-(1-methylpyrazol-4-yl)pyrazolo[1,5-a]pyrazin-4-yl]phenyl]prop-2-enamide (105 mg, 287.11 umol, 76.06% yield, 98% purity). LCMS: (M+H⁺: 359.1). ¹H NMR (500 MHz, DMSO-d₆) δ ppm 9.19 (s, 1H) 8.35 (s, 1H) 8.22 (d, J=2.44 Hz, 1H) 8.13 (s, 2H) 8.00 (s, 1H) 7.70 (t, J=7.94 Hz, 1H) 7.50-7.56 (m, 1H) 7.17 (dd, J=2.44, 1.22 Hz, 1H) 6.21 (br s, 2H) 5.59-5.68 (m, 1H) 3.91 (s, 3H) 3.35-3.39 (m, 3H).

### Examples 30 & 31: (R)-1-(4-((6-(1-methyl-1H-pyrazol-4-yl)pyrazolo[1,5-a]pyrazin-4-yl)thio)azepan-1-yl)prop-2-en-1-one and (S)-1-(4-((6-(1-methyl-1H-pyrazol-4-yl)pyrazolo[1,5-a]pyrazin-4-yl)thio)azepan-1-yl)prop-2-en-1-one

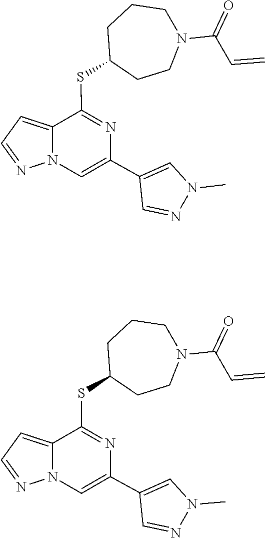 [SMILES: C=CC(=O)N1CCC[C@@H](Sc2nc(-c3cnn(C)c3)cn3nccc23)CC1 ; C=CC(=O)N1CCC[C@H](Sc2nc(-c3cnn(C)c3)cn3nccc23)CC1]

### 1. Synthesis of tert-butyl 4-((methylsulfonyl)oxy)azepane-1-carboxylate

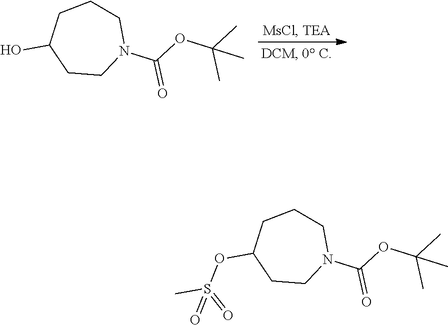 [SMILES: CC(C)(C)OC(=O)N1CCCC(O)CC1 ; CC(C)(C)OC(=O)N1CCCC(OS(C)(=O)=O)CC1]

TEA (201 mg, 1.99 mmol) was added dropwise to an ice-cold solution of tert-butyl 4-hydroxyazepane-1-carboxylate (214 mg, 0.994 mmol) in DCM (9.5 mL) under N₂. To this was added MsCl (168 mg, 1.47 mmol) at 0° C. and the resulting solution stirred at 0° C. for 2 h. The mixture was quenched by addition of water (10 mL) and the mixture was extracted with DCM (20 mL×3). The combined organics were washed with brine (10 mL), dried (MgSO₄) and evaporated to dryness in vacuo to give tert-butyl 4-((methylsulfonyl)oxy)azepane-1-carboxylate (0.326 g, yield: 112%) as colorless oil. ESI-MS (M+Na)⁺: 316.0.

### 2. Synthesis of tert-butyl 4-(acetylthio)azepane-1-carboxylate

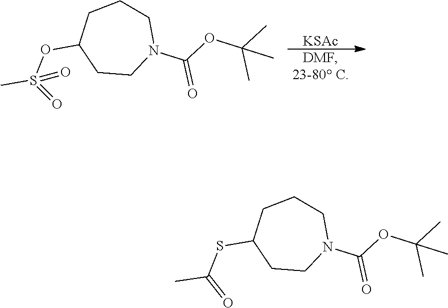

To a RT solution of tert-butyl 4-((methylsulfonyl)oxy)azepane-1-carboxylate (0.163 g, 0.52 mmol) in DMF (5.5 mL) was added potassium thioacetate (112 mg, 0.980 mmol) and the mixture stirred at 80° C. under nitrogen overnight. The mixture was quenched by addition of water (10 mL) and extracted with EtOAc (10 mL×3). The combined organic layers were washed with brine, dried (Na2SO₄) and concentrated in vacuo. The residue was purified by silica gel chromatography (0-100% EtOAc in heptanes) to give tert-butyl 4-(acetylthio)azepane-1-carboxylate (0.105 g, yield: 74%) as orange oil. ESI-MS (M+Na)⁺: 296.1.

### 3. Synthesis of tert-butyl 4-mercaptoazepane-1-carboxylate

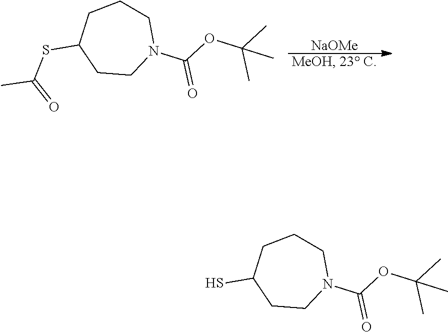 [SMILES: CC(=O)SC1CCCN(C(=O)OC(C)(C)C)CC1 ; "MeOH, 23° C." ; CC(C)(C)OC(=O)N1CCCC(S)CC1 ; "NaOMe"]

To a RT solution of tert-butyl 4-(acetylthio)azepane-1-carboxylate (0.104 g, 379 μmol) in MeOH (6 mL) was added sodium methoxide (120 uL, 0.52 mmol, 4.37 M) and the mixture stirred at RT under nitrogen for 1 h. The mixture was diluted with water (10 mL) and extracted with EtOAc (20 mL×3). The combined organics were washed with saturated sodium chloride solution, dried (Na2SO₄) and evaporated to dryness in vacuo to give tert-butyl 4-mercaptoazepane-1-carboxylate (83 mg, yield: 95%) as orange oil. ¹H NMR (400 MHz, CDCl₃) δ 3.47-3.62 (m, 1H), 3.15-3.47 (m, 3H), 3.00-3.13 (m, 1H), 1.58-2.19 (m, 7H), 1.47 (s, 9H).

### 4. Synthesis of tert-butyl 4-((6-(1-methyl-1H-pyrazol-4-yl)pyrazolo[1,5-a]pyrazin-4-yl)thio)azepane-1-carboxylate

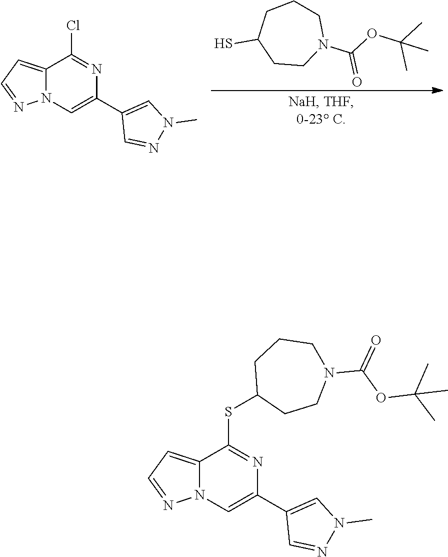 [SMILES: Cn1cc(-c2cn3nccc3c(Cl)n2)cn1 ; CC(C)(C)OC(=O)N1CCCC(S)CC1 ; Cn1cc(-c2cn3nccc3c(SC3CCCN(C(=O)OC(C)(C)C)CC3)n2)cn1]

To a stirred 0° C. solution of tert-butyl 4-mercaptoazepane-1-carboxylate (83 mg, 0.360 mmol) in THF (5 mL) was added sodium hydride (43 mg, 1.1 mmol, 60% purity) and the mixture stirred at 0° C. for 0.5 h before 4-chloro-6-(1-methyl-1H-pyrazol-4-yl)pyrazolo[1,5-a]pyrazine (101 mg, 0.433 mmol) was added and the resulting mixture stirred at RT for 2 h. The reaction was quenched by addition of water (2 mL) and extracted with EtOAc (3×5 mL). The combined organics were washed with saturated sodium chloride solution, dried (Na2SO₄) and evaporated to dryness in vacuo. The residue was purified twice by silica gel chromatography, first using 0-100% 3:1 EtOAc-EtOH in heptanes as eluent then using 0-10% MeOH in methylene chloride as eluent. The material additionally purified using reverse phase HPLC (Waters XSelect CSH C18, 5 μm, 50 mm×100 mm column with mobile phase H₂O (A) and MeCN (B) and a gradient of 10-90% B (0.1% TFA final v/v % modifier) with flow rate at 30 mL/min) to give tert-butyl 4-((6-(1-methyl-1H-pyrazol-4-yl)pyrazolo[1,5-a]pyrazin-4-yl)thio)azepane-1-carboxylate (76 mg, yield: 49%) as colorless oil. ESI-MS (M+H)⁺: 429.2.

### 5. Synthesis of 4-(azepan-4-ylthio)-6-(1-methyl-1H-pyrazol-4-yl)pyrazolo[1,5-a]pyrazine

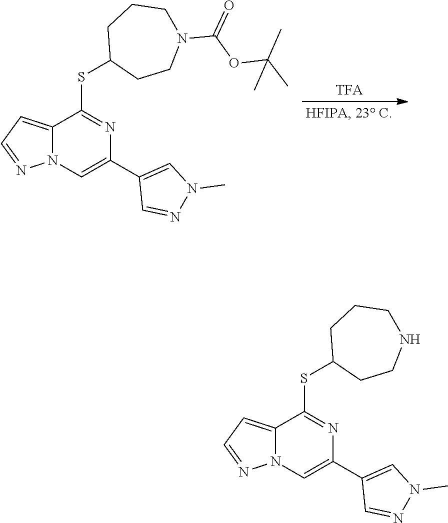 [SMILES: Cn1cc(-c2cn3nccc3c(SC3CCCN(C(=O)OC(C)(C)C)CC3)n2)cn1 ; Cn1cc(-c2cn3nccc3c(SC3CCCNCC3)n2)cn1]

TFA (60 mg, 530 μmol, 40 uL) was added to a solution of tert-butyl 4-((6-(1-methyl-1H-pyrazol-4-yl)pyrazolo[1,5-a]pyrazin-4-yl)thio)azepane-1-carboxylate (76 mg, 0.180 mmol) in HFIPA (1 mL) and the solution stirred at RT for 1 h. The reaction mixture was evaporated to dryness, diluted with DCM, and washed with saturated sodium bicarbonate solution. The aqueous solution was re-extracted with a mixture of CHCl₃/IPA (8 to 1 v/v ratio, 3 times). The combined organics were dried (MgSO₄) and evaporated to dryness in vacuo to give 4-(azepan-4-ylthio)-6-(1-methyl-1H-pyrazol-4-yl)pyrazolo[1,5-a]pyrazine (52 mg, yield: 90% yield) as a colorless film which was used without additional purification. ESI-MS (M+H)⁺: 329.1.

### 6. Synthesis of (R)-1-(4-((6-(1-methyl-1H-pyrazol-4-yl)pyrazolo[1,5-a]pyrazin-4-yl)thio)azepan-1-yl)prop-2-en-1-one and (S)-1-(4-((6-(1-methyl-1H-pyrazol-4-yl)pyrazolo[1,5-a]pyrazin-4-yl)thio)azepan-1-yl)prop-2-en-1-one

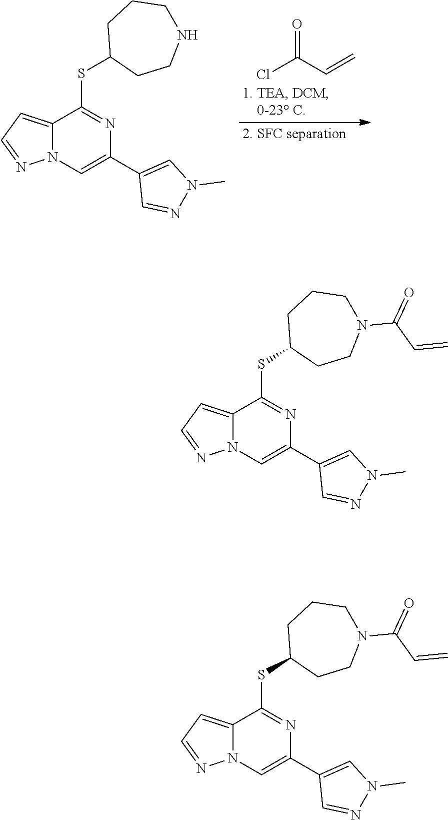

To a 20 mL vial containing 4-(azepan-4-ylthio)-6-(1-methyl-1H-pyrazol-4-yl)pyrazolo[1,5-a]pyrazine (52 mg, 0.160 mmol) was added DCM (1 mL) followed by triethylamine (80 mg, 0.79 mmol). The mixture was stirred for 5 mins at RT, then cooled to 0° C. Acryloyl chloride (21 mg, 0.240 mmol) was added dropwise and the reaction was allowed to slowly warm to RT. After stirring for 1 h at RT, the reaction mixture was diluted with DCM, washed with saturated sodium bicarbonate solution. The organic layer was collected. The aqueous layer was re-extracted 2×DCM. The combined organics were dried (MgSO₄) and evaporated to dryness in vacuo to give a pale yellow film. This material was purified by silica gel chromatography (0-100% 3:1 EtOAc-EtOH in heptanes) to give racemic 1-(4-((6-(1-methyl-1H-pyrazol-4-yl)pyrazolo[1,5-a]pyrazin-4-yl)thio)azepan-1-yl)prop-2-en-1-one (30 mg, yield: 49%) as a colorless film. 1-(4-((6-(1-methyl-1H-pyrazol-4-yl)pyrazolo[1,5-a]pyrazin-4-yl)thio)azepan-1-yl)prop-2-en-1-one (25 mg) was separated by chiral SFC (CHIRALPAK AD-H 30×250 mm, 5 μm, 45% MeOH with no modifier in CO₂, flow rate: 100 mL/min, ABPR 120bar, MBPR 40 psi, column temp 40° C.) to afford:

*Peak 1, Example 30, E1 (8.6 mg, 34%). 1H NMR (400 MHz, MEOH-d4) δ 8.55 (s, 1H), 8.08-8.18 (m, 1H), 7.99 (s, 1H), 7.94 (t, J=2.01 Hz, 1H), 6.82 (ddd, J=10.54, 15.18, 16.69 Hz, 1H), 6.72-6.75 (m, 1H), 6.30 (ddd, J=2.01, 11.29, 16.82 Hz, 1H), 5.78 (ddd, J=2.01, 10.54, 16.56 Hz, 1H), 4.25-4.45 (m, 1H), 3.98 (d, J=4.27 Hz, 3H), 3.83-3.93 (m, 1H), 3.57-3.83 (m, 3H), 2.37-2.55 (m, 1H), 2.17-2.28 (m, 1H), 1.88-2.17 (m, 4H). ESI-MS (M+H)⁺: 383.1.

*Peak 2, Example 31, E2 (9.2 mg, 37%). 1H NMR (400 MHz, MEOH-d4) δ 8.55 (s, 1H), 8.09-8.17 (m, 1H), 7.99 (s, 1H), 7.94 (t, J=2.01 Hz, 1H), 6.82 (ddd, J=10.54, 15.12, 16.75 Hz, 1H), 6.72-6.76 (m, 1H), 6.31 (ddd, J=2.01, 11.11, 16.75 Hz, 1H), 5.78 (ddd, J=2.13, 10.54, 16.69 Hz, 1H), 4.26-4.45 (m, 1H), 3.98 (d, J=4.27 Hz, 3H), 3.83-3.93 (m, 1H), 3.56-3.83 (m, 3H), 2.39-2.54 (m, 1H), 2.18-2.28 (m, 1H), 1.87-2.18 (m, 4H). ESI-MS (M+H)⁺: 383.1.

### D. In Vitro Assays

### In Vitro BTK Kinase Assay: Btk-PolyGAT-LS Assay

The purpose of the BTK in vitro assay is to determine compound potency against BTK through the measurement of IC50. Compound inhibition is measured after monitoring the amount of phosphorylation of a fluorescein-labeled polyGAT peptide (Invitrogen PV3611) in the presence of active BTK enzyme (Upstate 14-552), ATP, and inhibitor. The BTK kinase reaction was done in a black 96 well plate (costar 3694). For a typical assay, a 24 μL aliquot of a ATP/peptide master mix (final concentration; ATP 10 PM, polyGAT 100 nM) in kinase buffer (10 mM Tris-HCl pH 7.5, 10 mM MgCl2, 200 μM Na3PO4, 5 mM DTT, 0.01% Triton X-100, and 0.2 mg/ml casein) is added to each well. Next, I uL of a 4-fold, 40× compound titration in 100% DMSO solvent is added, followed by adding 15 uL of BTK enzyme mix in 1× kinase buffer (with a final concentration of 0.25 nM). The assay is incubated for 30 minutes before being stopped with 28 pL of a 50 mM EDTA solution. Aliquots (5 uL) of the kinase reaction are transferred to a low volume white 384 well plate (Corning 3674), and 5 pL of a 2× detection buffer (Invitrogen PV3574, with 4 nM Tb-PY20 antibody, Invitrogen PV3552) is added. The plate is covered and incubated for 45 minutes at room temperature. Time resolved fluorescence (TRF) on Molecular Devices M5 (332 nm excitation; 488 nm emission; 518 nm fluorescein emission) is measured. IC50 values are calculated using a four parameter fit with 100% enzyme activity determined from the DMSO control and 0% activity from the EDTA control.

Table 1 shows the activity of the selected exemplary compounds of this invention in the in vitro Btk kinase assay, wherein each compound number corresponds to the example numbers in Examples 1-31. “†” represents an IC₅₀ of greater than 1 μM and equal to or less than 10 μM. “††” represents an IC₅₀ of greater than 10 nM and equal to or less than 1 μM (10 nM<IC₅₀≤1 μM). “†††” represents an IC₅₀ of greater than 1 nM and equal to or less than 10 nM (1 nM<IC₅₀≤10 nM). “††††” represents an IC50 of less than 1 nM.

TABLE 1

| | In Vitro BTK |
|---|---|
| | Kinase Assay |
| Example | IC₅₀ Score |
| 1 | †††† |
| 2 | ††† |
| 3 | †††† |
| 4 | ††† |
| 5 | †† |
| 6 | †† |
| 7 | ††† |
| 8 | ††† |
| 9 | ††† |
| 10 | †† |
| 11 | ††† |
| 12 | †† |
| 13 | ††† |
| 14 | ††† |
| 15 | †† |
| 16 | ††† |
| 17 | †† |
| 18 | † |
| 19 | † |
| 20 | †† |
| 21 | ††† |
| 22 | †† |
| 23 | †† |
| 24 | †† |
| 25 | †† |
| 26 | ††† |
| 27 | †† |
| 28 | †† |
| 29 | ††† |
| 30 | †† |
| 31 | † |

### In Vitro Whole Blood CD69 Assay

Human heparinized venous blood from health donors was aliquoted into 96-well plate and “spiked” with serial dilutions of formula I compounds in DMSO or with DMSO without drug. The final concentration of DMSO in all wells was 0.1%. The plate was incubated at 37° C. for 30 min. Drug-containing samples were stimulated with 0.1 pg/mL mouse anti-human IgD-dextran (1A62) or 20 μg/mL polyclonal rabbit F(ab′)2 anti-human IgD.

Phosphate-buffered saline (PBS) was added to the negative control unstimulated sample and the plates were incubated overnight (18 to 22 hours) at 37° C. Cells were stained with fluorochrome-conjugated anti-CD19 and anti-CD69 antibodies. Lyse/fix solution was used to remove red blood cells by hypotonic lysis and to fix the remaining cells, which were then analyzed by flow cytometry. CD19+B cells were gated and analyzed for CD69 expression.

The percentage of B cells expressing CD69 was plotted versus the log 10 of the concentration of the drug and the best-fit curves (variable Hill slope) were generated to obtain the IC50 value.

Table 2 shows the activity of the selected exemplary compounds of this invention in the in vitro whole blood CD69 assay, wherein each compound number corresponds to the example numbering set forth in the Examples 1-31 herein. “†” represents an IC₅₀ of greater than 10 μM. “††” represents an IC₅₀ of greater than 1 μM and equal to or less than 10 μM (1 μM<IC₅₀≤10 μM). “†††” represents an IC₅₀ of less than 1 μM.

TABLE 2

| | In Vitro HWB |
|---|---|
| | CD69 Assay |
| Example | IC₅₀ Score |
| 1 | ††† |
| 2 | †† |
| 3 | ††† |
| 4 | †† |
| 5 | nt |
| 6 | †† |
| 7 | ††† |
| 8 | †† |
| 9 | †† |
| 10 | nt |
| 11 | †† |
| 12 | nt |
| 13 | †† |
| 14 | †† |
| 15 | †† |
| 16 | †† |
| 17 | nt |
| 18 | †† |
| 19 | ††† |
| 20 | nt |
| 21 | nt |
| 22 | nt |
| 23 | nt |
| 24 | †† |
| 25 | †† |
| 26 | †† |
| 27 | †† |
| 28 | †† |
| 29 | †† |
| 30 | nt |
| 31 | nt |

nt: not tested

1. A compound represented by formula (I): 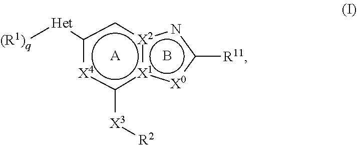
or a pharmaceutically acceptable salt thereof, wherein:
X⁰ is N, X¹ is C, X² is N and X⁴ is N; X⁰ is CR⁰, X¹ is C, X² is N and X⁴ is N; X⁰ is CR⁰, X¹ is N, X² is C and X⁴ is N; X⁰ is CR⁰, X¹ is N, X² is C and X⁴ is CH; or X⁰ is CR⁰, X¹ is C, X² is N and X⁴ is CH;
R⁰ is H, halo, —CH₃, halomethyl, cyclpropyl or CN;
Het is phenyl, a 5-6 membered heteroaryl or a N—(C₁-C₄ alkyl)pyridonyl;
R¹ is H or C₁-C₃ alkyl, C₁-C₃ alkoxy, C₁-C₃ haloalkyl, C₃-C₆ cycloalkyl, or a 4-7 membered monocyclic oxygen containing heterocycle;
X³ is absent, —S—, —SO₂—, CR^{3a}R^{3b}, —C(═O)— or —(C═O)—NH—*, where * indicates a point of attachment to R²
R^{3a} and R^{3b} are each independently H or halo, wherein at least one of R^{3a} and R^{3b} is not H;
when X³ is absent, —S—, —SO₂—, CR^{3a}R^{3b}, or —(C═O)—NH—*, R² is a 4-12 membered mono or bicyclic nitrogen-containing heterocycle bonded to X³ through a ring carbon atom (“C-attached”), an 8-12 membered bicyclic nitrogen-containing heterocycle bonded to X³ through a ring nitrogen atom (“N-attached”), a 4-7 membered monocyclic oxygen containing heterocycle, phenyl, or a 3-12 membered monocyclic or bicyclic carbocyclyl, wherein the 4-7 membered monocyclic oxygen containing heterocycle, the phenyl and the 3-12 membered monocyclic or bicyclic carbocyclyl represented by R² are each substituted with a group represented by R⁴ and optionally further substituted with one or two groups represented by R¹⁰; the C-attached 4-12 membered mono or bicyclic nitrogen-containing heterocycle represented by R² is N-substituted with a group represented by R⁵ and optionally further substituted with one or two groups represented by R¹⁰; and the N-attached 8-12 membered bicyclic nitrogen-containing heterocycle represented by R² is N-substituted with a group represented by R⁵ and optionally further substituted with one or two groups represented by R¹⁰;
when X³ is absent, R² can also be represented by formula (A): 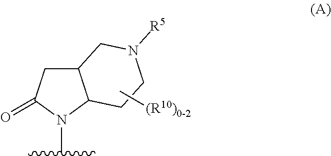
when X³ is —C(═O)—, R² is a 4-12 membered mono or bicyclic nitrogen-containing heterocycle bonded to X³ through a ring nitrogen atom (“N-attached”), a 4-7 membered monocyclic oxygen containing heterocycle, phenyl, or a 3-12 membered monocyclic or bicyclic carbocyclyl, wherein the 4-7 membered monocyclic oxygen containing heterocycle, the phenyl and the 3-12 membered monocyclic or bicyclic carbocyclyl represented by R² are each substituted with a group represented by R⁴ and optionally further substituted with one or two groups represented by R¹⁰; the N-attached 4-12 membered mono or bicyclic nitrogen-containing heterocycle represented by R² is C-substituted with a group represented by R⁴ and optionally further substituted with one or two groups represented by R¹⁰;
R⁴ is 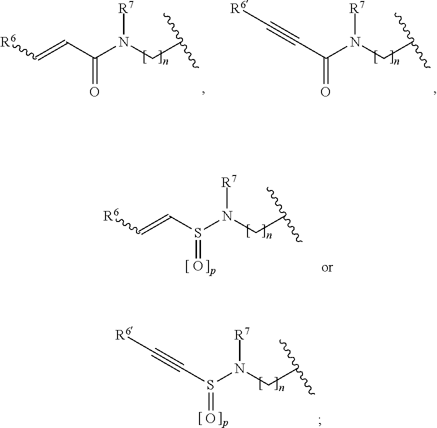
R⁵ is 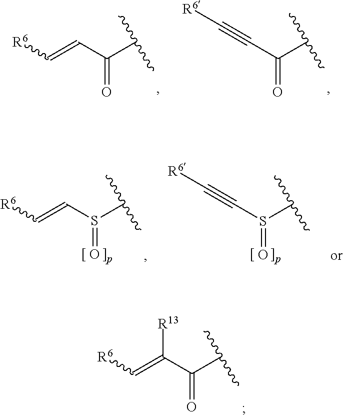
R⁶ is H, C₁-C₃ alkyl, C₁-C₃ haloalkyl, N(R^{a})₂ or CH₂N(R^{a})₂, wherein each R^{a} is independently H or methyl;
R^{6′} is H, C₁-C₃ alkyl or C₁-C₃ haloalkyl;
R⁷ is H, C₁-C₂ alkyl or C₁-C₂ fluoroalkyl;
each R¹⁰ is independently F or C₁₋₃alkyl;
R¹¹ is H or N(R¹²)₂;
each R¹² is independently H or C₁-C₃ alkyl;
R¹³ is CN or F;
n is 0 or 1;
p is 1 or 2; and
q is 1 or 2.

2. The compound of claim 1, or a pharmaceutically acceptable salt thereof, wherein R¹¹ is H.

3. The compound of claim 1, wherein the compound is represented by one of the following formula: 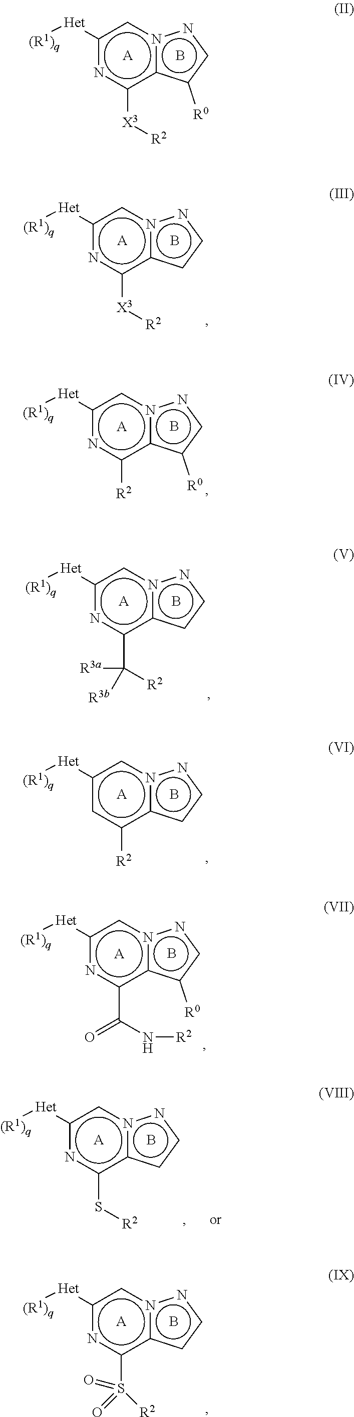
or a pharmaceutically acceptable salt thereof, wherein R^{3a} and R^{3b} are each independently H or halo, and at least one of R^{3a} and R^{3b} is not H.

4. (canceled)

5. The compound of claim 3, or a pharmaceutically acceptable salt thereof, wherein R⁰ is H, C₁, F or —CH₃; and R^{3a} and R^{3b} are each F.

6. (canceled)

7. The compound of claim 1, or a pharmaceutically acceptable salt thereof, wherein:
R² is selected from:
(i) cyclobutanyl, cyclopentanyl, cyclohexanyl and phenyl, each of which is substituted with a group represented by R⁴ and is optionally further substituted with one or two groups represented by R¹⁰;
(ii) azepanyl, azetidinyl, 9-azabicyclo[3.3.1]nonanyl, 2-azabicyclo[2.2.2]octanyl, 8-azabicyclo[3.2.1]octanyl, 2,7-diazaspiro[4.4]nonane, octahydrocyclopenta[c]pyrrolyl, octahydro-1H-pyrrolo[3,4-c]pyridine, piperidinyl, tetrahydropyridinyl and pyrrolidinyl, each of which is N-substituted with the group represented by R⁵ and optionally further substituted with the one or two groups represented by R¹⁰;
(iii) 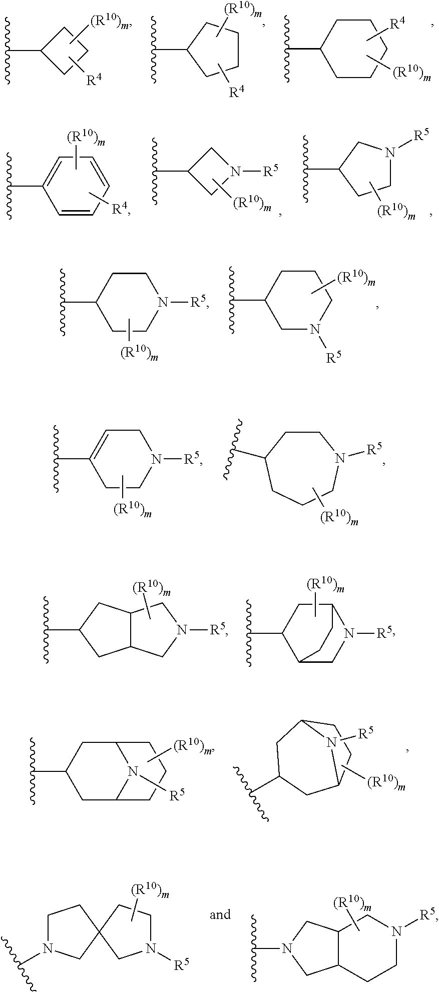
wherein m is 0, 1 or 2, and 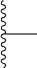
represents a bond to X or ring A; or
(iv) 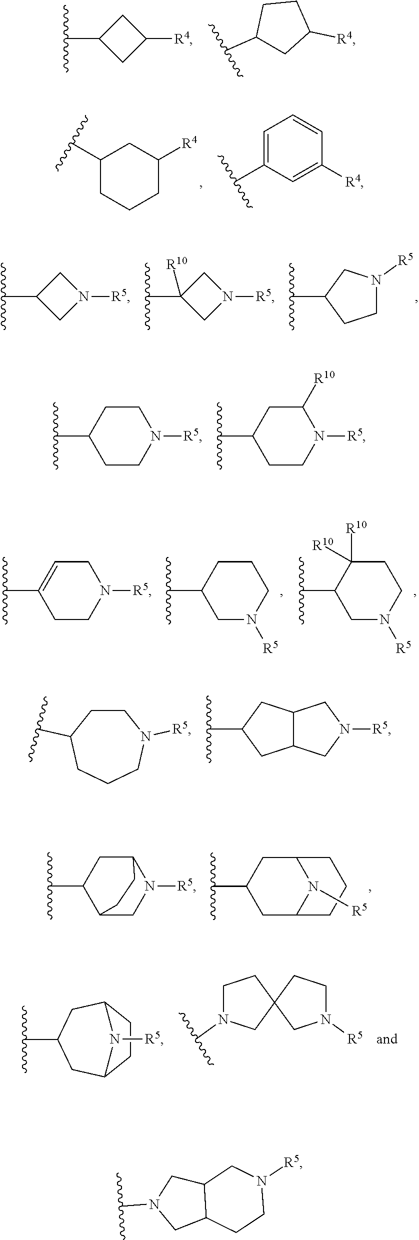
wherein 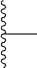
represents a bond to X³ or ring A.

8-9. (canceled)

10. The compound of claim 1, wherein the compound is represented by the following formula: 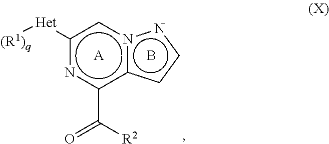
or a pharmaceutically acceptable salt thereof.

11. The compound of claim 10, or a pharmaceutically acceptable salt thereof, wherein R² is selected from:
(i) a 4-7 membered monocyclic nitrogen-containing heterocycle bonded to X³ through a ring nitrogen atom (“N-attached”) and a 4-6 membered monocyclic carbocyclyl, wherein the 4-6 membered monocyclic carbocyclyl represented by R² are each substituted with a group represented by R⁴ and optionally further substituted with one or two groups represented by R¹⁰; and the N-attached 4-7 membered monocyclic nitrogen-containing heterocycle represented by R² is C-substituted with a group represented by R⁴ and optionally further substituted with one or two groups represented by R¹⁰;
(ii) 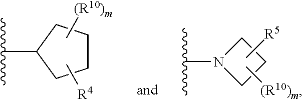
wherein m is 0, 1 or 2, and 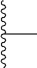
represents a bond to C(O)-ring A; or
(iii) 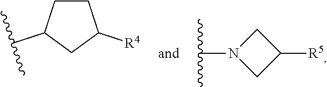
wherein 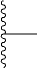
represents a bond to C(O)-ring A.

12-13. (canceled)

14. The compound of claim 11, or a pharmaceutically acceptable salt thereof, wherein each R¹⁰ is independently F, —CH₃ or —CH₂CH₃.

15. The compound of claim 1, or a pharmaceutically acceptable salt thereof,
wherein:
R⁴ is 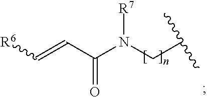
R⁵ is: 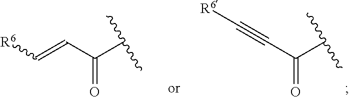
R⁶ is H or —CF₃;
R^{6′} is —CH₃;
R⁷ is H, —CH₃ or —CH₂CH₃;
n is 0 or 1, and 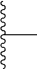
represents a bond to R².

16. The compound of claim 1, or a pharmaceutically acceptable salt thereof, wherein Het is 5 membered heteroaryl, preferably, Het is:
(i) pyrazolyl;
(ii) N 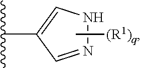
wherein 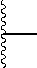
represents a bond to ring A; or
(iii) 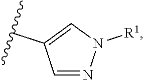
wherein 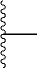
represents a bond to ring A.

17-19. (canceled)

20. The compound of claim 1, or a pharmaceutically acceptable salt thereof, wherein R¹ is C₁-C₃ alkyl, C₁-C₃ haloalkyl or C₃-C₆ cycloalkyll, preferably, R¹ is —CH₃, —CF₃, cyclopropyl, or cyclobutyl.

21. (canceled)

22. The compound of claim 1, wherein the compound is represented by one of the following formula: 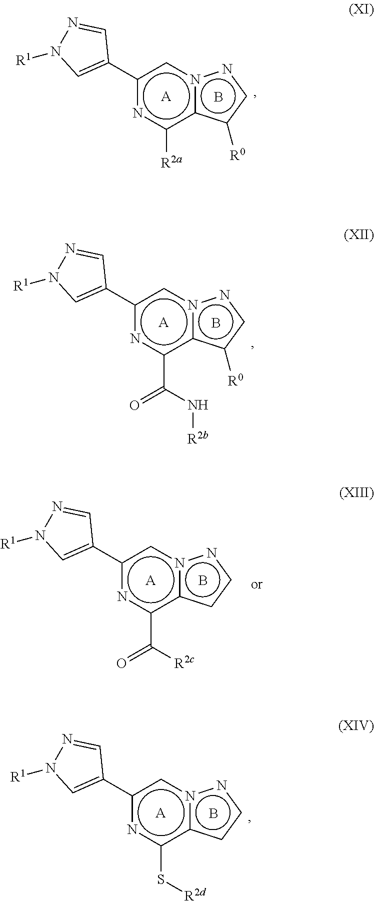
or a pharmaceutically acceptable salt thereof, wherein:
R⁰ is H, F, or —CH₃;
R¹ is —CH₃, cyclopropyl or cyclobutyl;
R^{2a} is a 6-9 membered mono or bicyclic nitrogen-containing heterocycle bonded to X³ through a ring carbon atom (“C-attached”) which is N-substituted with a group represented by R⁵ and optionally further substituted with one or two groups represented by R¹⁰;
R^{2b} is a 4-6 membered monocyclic nitrogen-containing heterocycle bonded to X³ through a ring carbon atom (“C-attached”) which is N-substituted with a group represented by R⁵ and optionally further substituted with one or two groups represented by R¹⁰;
R^{2c} is a C₃₋₆cycloalkyl which is substituted with a group represented by R⁴ and optionally further substituted with one or two groups represented by R¹⁰;
R^{2d} is a 4-7 membered monocyclic nitrogen-containing heterocycle bonded to X³ through a ring carbon atom (“C-attached”) which is N-substituted with a group represented by R⁵ and optionally further substituted with one or two groups represented by R¹⁰, or a C₃₋₆cycloalkyl which is substituted with a group represented by R⁴ and optionally further substituted with one or two groups represented by R¹⁰;
R⁴ is 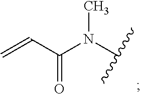 [SMILES: *N(C)C(=O)C=C]
R⁵ is 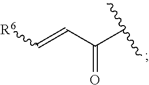
and
R⁶ is H or CF₃.

23. The compound of claim 22, or a pharmaceutically acceptable salt thereof, wherein:
R^{2a} is 8-azabicyclo[3.2.1]octanyl, octahydrocyclopenta[c]pyrrolyl, or piperidinyl, each of which is N-substituted with the group represented by R⁵ and optionally further substituted with the one or two groups represented by R¹⁰;
R^{2b} is azetidinyl or pyrrolidinyl, each of which is N-substituted with the group represented by R⁵ and optionally further substituted with the one or two groups represented by R¹⁰;
R^{2c} is cyclopentyl substituted with a group represented by R⁴ and optionally further substituted with one or two groups represented by R¹⁰; and
R^{2d} is azepanyl N-substituted with a group represented by R⁵ and optionally further substituted with one or two groups represented by R¹⁰, or cyclobutyl substituted with a group represented by R⁴ and optionally further substituted with one or two groups represented by R¹⁰.

24. The compound of claim 22, or a pharmaceutically acceptable salt thereof, wherein:
R^{2a} is 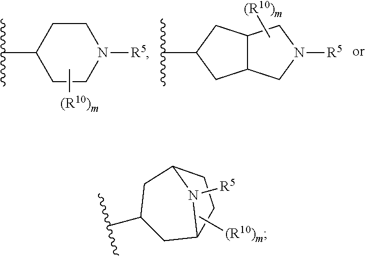
R^{2b} is 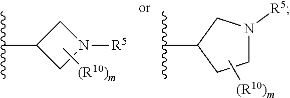
R^{2c} is 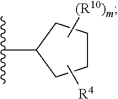
and
R^{2d} is 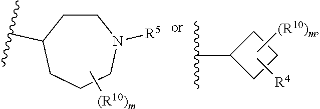
wherein m is 0, 1 or 2, and 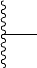
represents a bond to X³ or ring A.

25. The compound of claim 22, or a pharmaceutically acceptable salt thereof, wherein:
R^{2a} is 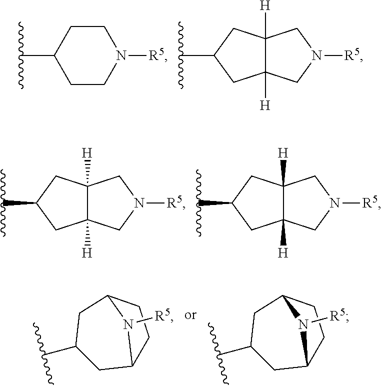
R^{2b} is 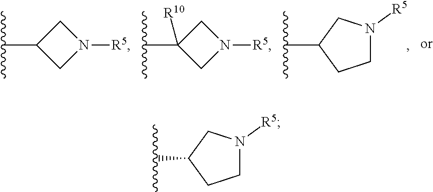
R^{2c} is 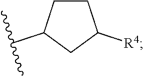
and
R^{2s} is 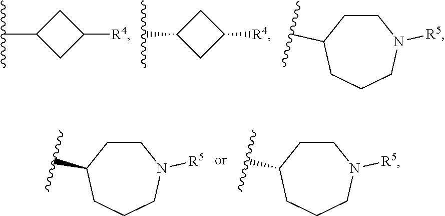
wherein R¹⁰ is —CH₃ or —CH₂CH₃, and 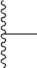
represents a bond to X³ or ring A.

26. A pharmaceutical composition comprising a compound of claim 1 or a pharmaceutically acceptable salt thereof, and a pharmaceutically acceptable excipient.

27. A method of treating a disorder responsive to inhibition of Bruton's tyrosine kinase in a subject comprising administering to the subject an effective amount of the compound according to claim 1, or a pharmaceutically acceptable salt thereof.

28. The method of claim 27, wherein the disorder is an autoimmune disorder, atopic dermatitis, leukemia or lymphoma.

29. The method of claim 28, wherein the autoimmune disorder is rheumatoid arthritis, systemic lupus erythematosus.

30-32. (canceled)

33. The method of claim 28, wherein the autoimmune disorder is multiple sclerosis.
